(19) United States
(12) Patent Application Publication
(10) Pub. No.: US 2023/0123497 A1
(43) Pub. Date: Apr. 20, 2023

(54) NOVEL ANTIBACTERIAL HYDROGELS

(71) Applicant: NewSouth Innovations Pty Limited, Sydney, New South Wales (AU)

(72) Inventors: Vina Rizki Aldilla, Sydney, New South Wales (AU); Naresh Kumar, Sydney, New South Wales (AU); Mark Duncan Perry Willcox, Sydney, New South Wales (AU); David StClair Black, Sydney, New South Wales (AU); Renxun Chen, Sydney, New South Wales (AU)

(21) Appl. No.: 18/001,936

(22) PCT Filed: Jun. 17, 2021
(86) PCT No.: PCT/AU2021/050628

§ 371 Date: Dec. 15, 2022

(30) Foreign Application Priority Data
Jun. 17, 2020 (AU) 2020902013

(51) Int. Cl. A01N 63/50; C07K 5/087; A01P 1/00; A01N 25/04; A61L 26/00; A61L 27/52; A61L 27/22; A61L 27/54; A01N 1/02; C09D 177/04

(52) U.S. Cl. CPC A01N 63/50; C07K 5/0812; A01P 1/00; A01N 25/04; A61L 26/008; A61L 27/52; A61L 27/227; A61L 26/0047; A61L 27/54; A61L 26/0066; A01N 1/0226; C09D 177/04; A61K 38/00

(57) ABSTRACT

The present invention relates to compounds of Formula I which form hydrogels upon mixing with water, and to fibers which form from the compounds. The hydrogels and fibers are antibacterial and not toxic towards mammalian cells. Such compounds, hydrogels, and fibers are useful, for example, in the treatment of surfaces such as in dermal or internal wounds as a barrier layer, or any article which may require disinfection. (I)

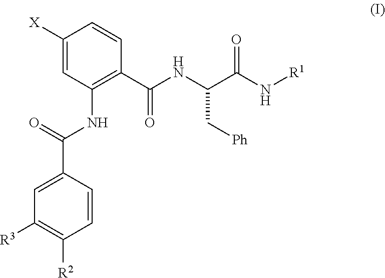

### TECHNICAL FIELD

The present invention relates to compounds which form hydrogels upon mixing with water, and to fibers which form from the compounds. The hydrogels and fibers are antibacterial and not toxic towards mammalian cells. Such compounds, hydrogels, and fibers are useful, for example, in the treatment of surfaces such as in dermal or internal wounds as a barrier layer, or any article which may require disinfection.

### BACKGROUND

Bacterial infections, whether due to cross-contamination from an infected surface or associated with wounds, are a great concern to human health. For example, the presence of Staphylococcus aureus (S. aureus), a Gram-positive bacterium and the most common causative pathogen in infections originating from hospitals (nosocomial infections), in wounds, can prolong wound healing through the release of toxins. In addition, severe bacterial infections can induce tissue morbidity, which might result in sepsis. Gram-negative bacteria such as Escherichia coli (E. coli) are also a major cause of serious food poisoning. For these reasons, extensive efforts have been devoted to developing alternative antibacterial agents and their formulations.

Hydrogels have been studied as a promising material for biomedical applications. Hydrogels are self-assembled supramolecular assemblies, comprising mostly water, which is held together by molecular networks. A hydrogel provides a physical barrier to prevent penetration of bacteria to a wound site. Due to their high water content, hydrogels also provide a well-hydrated environment which could accelerate the healing process. Nevertheless, this hydrated environment could also promote bacterial growth. Hence, hydrogels with antibacterial properties are more desirable.

The most common approach to generate an antibacterial hydrogel is to load the hydrogel with an active agent, such as an antibiotic, silver ions, or salicylic acid. However, these hydrogels could raise concerns associated with loading capacity, undesired burst release of the active agent, and biodegradability. However, hydrogels could also be endowed with antibacterial functions if they are assembled from hydrogelators with intrinsic antibacterial properties. Hydrogels with inherent antibacterial properties may be more desirable as they will exhibit self-delivery as well as minimise concerns about loading capacity or leaching of antibacterial agents that are simply mixed with the hydrogel. For topical applications, hydrogels have been described as being able to reduce discomfort during wound treatment, owing to their high water content which provides a moist environment, and the ability to allow oxygen absorption.

The majority of hydrogels with intrinsic antibacterial properties, to date, are based on cationic polymers or antimicrobial peptide (AMP) motifs. AMP's are typically 12 to 50 amino acids in length. However, the complicated synthesis and purification steps of these hydrogels usually limit their large-scale production.

Another way to provide a hydrogel with intrinsic antibacterial properties is to form a hydrogel from a compound that is able to release nitric oxide (NO) under physiological conditions. Nitric oxide (NO) is a short-lived, heterodiatomic molecule that is endogenously generated as a product of the oxidation of L-arginine to L-citrulline by nitric oxide synthase (NOS). Depending on its concentration, NO exerts a variety of biological functions such as anti-restenosis, anti-cancer, wound healing, and antibacterial activities.

The antibacterial function of NO can be attributed to various mechanisms. NO, by itself, exhibits nitrosative and oxidative effects. Furthermore, upon reaction with oxygen, superoxide, or hydrogen peroxide, NO can form reactive nitrogen species (RNS) such as peroxynitrite, nitrogen dioxide, dinitrogen trioxide, and dinitrogen tetroxide. These RNSs induce DNA damage, or inhibit enzyme function or lipid peroxidation, which leads to bacterial cell death. Owing to its multiple mechanisms of action, there is a lower chance of bacteria developing resistance to NO. However, elevated concentrations of NO could lead to undesirable effects such as apoptosis or structural atrophy of human cells. Therefore, to achieve the desired antibacterial function with low toxicity, the concentration of NO needs to be precisely regulated.

Nitric oxide donors (NO-donors) are molecules that can produce NO exogenously. Nitrobenzene, as a class of NO-donor, can generate NO through nitro-to-nitrate rearrangements in the presence of light. These derivatives usually contain substituents (namely CF₃, methyl, or arenes) at the ortho-position of nitrobenzene, which induces a twisted conformation of the nitro group. The twisted conformation allows nitro-to-nitrate photo rearrangement, due to overlap of p-orbitals of the nitro group oxygen with p-orbitals of the aromatic ring, which is followed by cleavage of the O—NO bond that generates NO.

Nitrobenzene derivatives are generally stable under physiological conditions. In addition, the use of light as a non-invasive trigger to release NO from these derivatives is beneficial since rapid and precise delivery can be achieved without affecting physiological parameters such as pH, temperature, and ionic strength.

Furthermore, touching contaminated then non-contaminated surfaces is a major source of transmission of bacteria in both hospitals as well as in the general public. To disinfect hands, the Centers for Disease Control and Prevention recommends washing hands thoroughly with soap and water, or use of an alcohol-based hand sanitizer comprising at least 60% alcohol. However, alcohols such as ethanol and isopropanol which are used in most hand sanitizers, quickly evaporate, at which point they are no longer effective. In addition, alcohol-based hand sanitizers are not as effective against some bacteria, such as Cryptosporidium and Clostridioides difficile, such that washing hands with soap and water is preferred under most circumstances.

There is a need for materials such as antibacterial hydrogels, or compositions comprising compounds that have intrinsic antibacterial properties, that mitigate at least one of the problems outlined above, for example, being effective against one or more types of bacteria, under a range of physiological conditions, are low-cost, or are not toxic to mammalian cells.

The present invention is predicated at least in part on the discovery that short peptides containing a cationic moiety or an NO-releasing moiety have antibacterial properties, and under certain conditions may form fibers and hydrogels, which may provide an effective alternative to the solutions presently available.

### SUMMARY

In one aspect, the present invention provides a compound of formula (I):

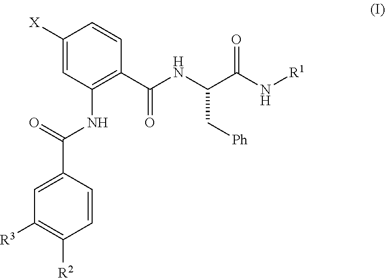

wherein:

R¹ is

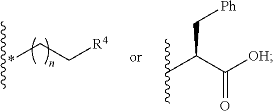

R⁴ is

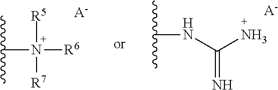

R⁵, R⁶, and R⁷ are, independently, H or CH₃;

A⁻ is Cl⁻, Br⁻, I⁻, CH₃C(O)O⁻, CF₃C(O)O⁻, or GdL⁻;

GdL⁻ is

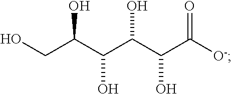

X is H, F, Cl, or Br;

R² is NO₂;

R³ is CH₃ or CF₃; or

R² and R³, when taken together with the carbon atoms to which they are attached, form a benzene ring, wherein the resulting naphthalene is optionally substituted with a group selected from the group consisting of halo, CN, CF₃, OCF₃, OCH₃, OCH₂CH₃, N(CH₃)₂, N(CH₂CH₃)₂, N(CH₃)(CH₂CH₃), COCH₃, COCH₂CH₃, OCOCH₃, SCH₃, SCH₂CH₃, C₁-C₄ alkyl, C₂-C₄ alkenyl, and C₂-C₄ alkynyl; and

n is 1, 2 or 3.

In another aspect, the present invention provides an antibacterial hydrogel, comprising water and a compound of formula (I):

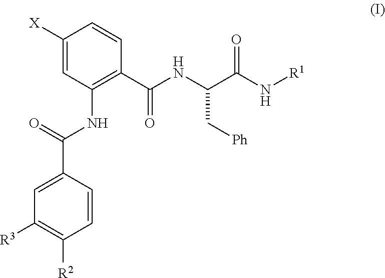

wherein:

R¹ is

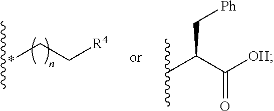

R⁴ is

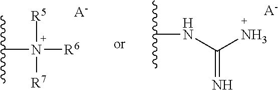

R⁵, R⁶, and R⁷ are, independently, H or CH₃;

A⁻ is Cl⁻, Br⁻, I⁻, CH₃C(O)O⁻, CF₃C(O)O⁻, or GdL⁻;

GdL⁻ is

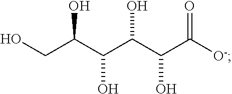

X is H, F, Cl, or Br⁻;

R² is NO₂;

R³ is CH₃ or CF₃; or

R² and R³, when taken together with the carbon atoms to which they are attached, form a benzene ring, wherein the resulting naphthalene is optionally substituted with a group selected from the group consisting of halo, CN, CF₃, OCF₃, OCH₃, OCH₂CH₃, N(CH₃)₂, N(CH₂CH₃)₂, N(CH₃)(CH₂CH₃), COCH₃, COCH₂CH₃, OCOCH₃, SCH₃, SCH₂CH₃, C₁-C₄ alkyl, C₂-C₄ alkenyl, and C₂-C₄ alkynyl; and

n is 1, 2 or 3.

Other aspects of the present invention will become apparent to those ordinarily skilled in the art upon review of the following description of specific embodiments of the invention.

### FIGURES

For a further understanding of the aspects and advantages of the present invention, reference should be made to the following detailed description, taken in conjunction with the accompanying drawings.

FIG. 1 shows results of vial inversion tests on nitrobenzene-appended short peptide 4: (a) at pH 11 in the absence of NaCl; (b) at pH 11 after addition of NaCl; and (c) at pH 5.5-6 after addition of NaCl.

FIG. 2 shows results of vial inversion tests on cationic peptides 10 and 11: (a) after heating; (b) upon addition of NaCl, after 10 minutes; and (c) upon addition of NaCl, after 24 hours.

FIG. 3 shows ¹H NMR of anthranilamide-based short cationic peptide 11 at its solution (Sol), viscous solution (VS), and hydrogel phase.

FIG. 4 shows ¹H NMR of nitrobenzene-appended short peptide 4 under various conditions.

FIG. 5 shows concentration-dependent UV-Vis spectra of short cationic peptide 11 which displays a bathochromic shift from 234-240 nm and elevation of shoulder peaks at 250-310 nm.

FIG. 6 shows circular dichroism (CD) spectra of anthranilamide-based short cationic peptides: (a) 11-13; (b) 15-17; and (c) 18 and 19, showing characteristic signals for random/disorder coil structures.

FIG. 7 shows CD spectra of hydrogels made from nitrobenzene-appended short peptide 4 at 0.47 μM using various triggers.

FIG. 8 shows frequency sweep test (FST) of: (a) 1° ammonium 11, (b) 3° ammonium 12, (c) 4° ammonium 13, (d) fluoro 15, (e) chloro 16, (f) bromo 17, (g) 3° ammonium bearing trifluoro acetate (TFA⁻) 18, and (h) 3° ammonium bearing Cl⁻ 19. Modulus storage (G′) are the grey data points; modulus loss (G″) are the black data points. The graphs represent the average of three individual measurements.

FIG. 9 shows FST of nitrobenzene-appended hydrogels 4 formed at (a) high pH; and (b) low pH, showing characteristics of stable hydrogels; strain sweep test (SST) of nitrobenzene-appended hydrogels 4 formed at (c) high pH; and (d) low pH. Modulus storage (G′) is denoted with lighter data points and modulus loss (G″) is denoted with darker data points.

FIG. 10 shows atomic force microscopy (AFM) images showing distinct fiber morphology in short peptide 11 (a) as a viscous solution at 0.05% w/v showing lack of junction zones, and (b) at a concentration of 4× below the CGC.

FIG. 11 shows AFM images showing overall fiber morphology of hydrogels made at a concentration of 4× below their CGC (a) primary ammonium 11 and (b) tertiary ammonium 12. Thick bundles, indicated with white arrows, were observed from (c) primary ammonium 11 and (d) tertiary ammonium 12. At e) is shown an AFM image of quaternary ammonium 13 at a concentration of 4× below the CGC.

FIG. 12 shows AFM images showing 3D networks imaged from hydrogels made from 3° ammonium compounds at a concentration of 4× below their CGC: (a) fluoro 15, (b) chloro 16, (c) bromo 17, (d) trifluoroacetate 18, and (e) chloride 19. Images were obtained at 0.025% w/v for all hydrogels, except bromo 17 which was imaged at 0.10% w/v.

FIG. 13 shows AFM images of hydrogels made from nitrobenzene-appended short peptide 4 at high pH (left) and low pH (right).

FIG. 14 shows antibacterial activity of anthranilamide-based cationic hydrogels in contact with S. aureus for 24 hours. n=2; *p<0.003 compared to negative control, ns=no significant difference to negative control. An anthranilamide-based hydrogel bearing acetyl group was used as the non-active gel.

FIG. 15 shows an antibacterial assay performed with nitrobenzene-appended anthranilamide-based hydrogel 4 against E. coli K12.

FIG. 16 shows viability of E. coli observed after treatments for 1 hour, showing significant bacteria reduction only from hydrogel 4 with blue light irradiation. (*p<0.0005 compared to other samples).

FIG. 17 shows viability of HEK 293T cells after exposure to hydrogels comprising primary ammonium 11, tertiary ammonium 12, quaternary ammonium 13, and chloro 16 groups at different concentrations.

FIG. 18 shows viability of HEK 293T cells after exposure to hydrogels made from nitrobenzene-appended short peptide 4 at 1% w/v and 2% w/v.

FIG. 19 shows UV-Vis spectrum of nitrobenzene-appended short peptide 4 at pH 5.

FIG. 20 shows cumulative release of NO from nitrobenzene-appended hydrogel 4 at 1% w/v via UV irradiation (λ=356 nm; circles) and blue light irradiation (λ=440-450 nm; squares) over 2 hours. Dotted line indicates the required concentration of NO (5 μM) to kill E. coli.

FIG. 21 shows results from testing hydrogel 4 after blue light irradiation for 2 hours (a) vial inversion test; and (b) frequency sweep test.

FIG. 22 shows AFM images of nitrobenzene-appended short peptide hydrogel 4 (a) before and (b) after irradiation of blue light (λ=440-450 nm).

FIG. 23 shows antibacterial activity of anthranilamide-based cationic hydrogel 11 in contact with E. coli, showing notable bacteria reduction (6.2 Log₁₀), compared to the non-active hydrogel which used as a control. n=3, p<0.0001.

FIG. 24 shows cumulative release of primary ammonium 11 from the hydrogel, at an initial concentration of 1% w/v.

FIG. 25 shows (a) fibers released from hydrogel 11; (b) antibacterial activity of anthranilamide-based cationic monomer, versus fiber, in contact with S. aureus; and (c) AFM image of ultra-short cationic peptide 11 at a concentration of 221 μM, prepared with 5% DMSO:water. Scale bar of AFM images is 1 μm.

### DETAILED DESCRIPTION

### Definitions

The following are some definitions that may be helpful in understanding the description of the present invention. These are intended as general definitions and should in no way limit the scope of the present invention to those terms alone, but are put forth for a better understanding of the following description.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, a number of terms are defined below.

The articles “a” and “an” are used herein to refer to one or to more than one (i.e. to at least one) of the grammatical object of the article. By way of example, “an element” means one element or more than one element.

As used herein, the term “about” encompasses small variations in the amount of the component being referred to. In this regard, a quantity, level, value, dimension, size, or amount that varies by as much as 15% or 10% to a reference quantity, level, value, dimension, size, or amount may be tolerated, in keeping with the spirit of the invention.

The term “short”, in relation to the term peptide, means that the compound is a peptide-like compound, and contains only one or two amino acids, along with other functional groups.

The term “antibacterial” means that the substance (compound, hydrogel, fiber, etc) is able to kill bacteria. Bacteria include Gram-positive and Gram-negative bacteria.

The term “hydrogel” means a self-assembled, supramolecular network comprising a compound and water, which is able to maintain a non-liquid phase.

The term “bacterial reduction” means the quantity of bacteria that is decreased, compared to a control sample. Large numbers are expressed as their logarithm to base 10 (Log₁₀). The quantity of bacteria in a sample is typically measured in CFUs/mL (CFU=colony-forming unit).

The term “% w/v” means a concentration of a substance when expressed as a percentage, i.e. % w/v=(weight in grams divided by volume in litres)×100.

The term “G′/G″ ratio” means the value obtained by dividing G′ (modulus storage) by G″ (modulus loss). G′ represents the elastic proportion (solid-state), whereas G″ represents the viscous proportion (liquid-state) of a viscoelastic material. G′ and G″ values can be obtained using a frequency sweep test (FST).

The term “linear viscoelastic region” or “LVER” means the range where increase of strain level does not affect the mechanical properties of a hydrogel. For topical applications, hydrogels which display larger LVER are preferred as they are more resistant to an applied strain, and hence could tolerate movement of the host without disintegrating.

The term “critical gel concentration” (CGC) is defined as the minimum mass of a compound required to provide a self-supporting hydrogel per unit volume in a vial inversion test (measured as % w/v).

The term “administering” and variations of that term including “administer” and “administration”, includes contacting, applying, delivering or providing a compound or composition of the invention to an organism, or a surface by any appropriate means.

In the context of this specification, the term “treatment”, refers to any and all uses which remedy a disease state or symptoms, prevent the establishment of disease, or otherwise prevent, hinder, retard, or reverse the progression of disease or other undesirable symptoms in any way whatsoever.

In the context of this specification the term “topical administration” or variations on that term including “topical application” includes within its meaning applying, contacting, delivering or providing a compound or composition of the invention to the skin, or localized regions of the body.

In the context of this specification the term “effective amount” includes within its meaning a sufficient but non-toxic amount of a compound or composition of the invention to provide a desired effect. Thus, the term “therapeutically effective amount” includes within its meaning a sufficient but non-toxic amount of a compound or composition of the invention to provide the desired therapeutic effect. The exact amount required will vary from subject to subject depending on factors such as the species being treated, the sex, age and general condition of the subject, the severity of the condition being treated, the particular agent being administered, the mode of administration, and so forth. Thus, it is not possible to specify an exact “effective amount”. However, for any given case, an appropriate “effective amount” may be determined by one of ordinary skill in the art using only routine experimentation.

The present invention relates to compounds which, when mixed with water, form a hydrogel which has antibacterial properties. The hydrogel does not require addition of any further anti-bacterial agents to produce the desired effect of killing bacteria, as the compounds forming the hydrogel have anti-bacterial properties.

A hydrogel may form spontaneously upon mixing of a hydrogelator compound with water. In some embodiments, a trigger is required to form the hydrogel, and there are many known triggers in the art. Triggers include cooling from an elevated temperature (temperature switch), changing the pH (pH switch), changing solvents (solvent switch), or the addition of salts.

In one aspect, the present invention provides a compound of formula (I):

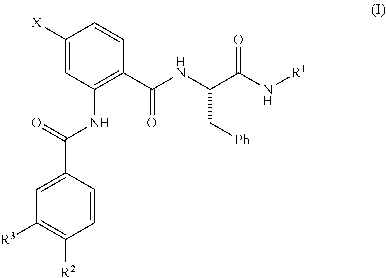

wherein:

R¹ is

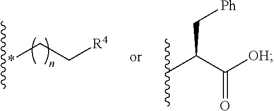

R⁴ is

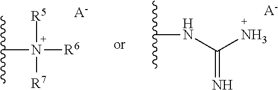

R⁵, R⁶, and R⁷ are, independently, H or CH₃;

A⁻ is Cl⁻, Br⁻, I⁻, CH₃C(O)O⁻, CF₃C(O)O⁻, or GdL⁻;

GdL⁻ is

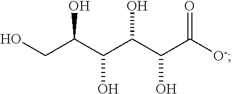

X is H, F, Cl, or Br;

R² is NO₂;

R³ is CH₃ or CF₃; or

R² and R³, when taken together with the carbon atoms to which they are attached, form a benzene ring, wherein the resulting naphthalene is optionally substituted with a group selected from the group consisting of halo, CN, CF₃, OCF₃, OCH₃, OCH₂CH₃, N(CH₃)₂, N(CH₂CH₃)₂, N(CH₃)(CH₂CH₃), COCH₃, COCH₂CH₃, OCOCH₃, SCH₃, SCH₂CH₃, C₁-C₄ alkyl, C₂-C₄ alkenyl, and C₂-C₄ alkynyl; and

n is 1, 2 or 3.

In some embodiments, R¹ is

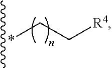

and R² and R³, when taken together with the carbon atoms to which they are attached, form a benzene ring. These embodiments can be described as cationic short peptides as the R⁴ group contains a cationic ammonium ion, along with a counter-anion.

These short peptides exert their anti-bacterial activity without requiring activation by light. R⁵, R⁶, and R⁷ are, independently of each other, either a hydrogen atom or a methyl group. All combinations of R⁵, R⁶, and R⁷ are expected to display antibacterial activity. For example, R⁵, R⁶ and R⁷ are hydrogen, or R⁵ and R⁶ are hydrogen and R⁷ is methyl, or R⁵ is hydrogen and R⁶ and R⁷ are methyl, or R⁵, R⁶ and R⁷ are methyl. The skilled person will recognise that, for all intents and purposes, R⁵, R⁶ and R⁷ are all equivalent, such that an embodiment wherein for example R⁵ and R⁶ are hydrogen and R⁷ is methyl is equivalent to when R⁵ and R⁷ are hydrogen and R⁶ is methyl. In one preferred embodiment, R⁵, R⁶, and R⁷ are hydrogen. In another embodiment, R⁵ is hydrogen and R⁶ and R⁷ are both methyl. In another embodiment, R⁵, R⁶, and R⁷ are all methyl.

In some embodiments, R⁴ is

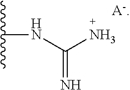

These compounds contain a guanidinium group, and are expected to form hydrogels under the right conditions, and with the right combination of other functional groups such as linker length, halogen substitution on the anthranilamide benzene ring, functionalisation on the naphthalene group, or the appropriate counter-anion.

The counter-anion (A⁻) to the above cationic short peptides can be any suitable negatively charged counter ion. Suitable examples include chloride, bromide, iodide, acetate, trifluoroacetate or GdL⁻. GdL or glucono-δ-lactone, is the lactone of gluconic acid, and is used in the food industry as an additive (E575). In water, GdL partially hydrolyses to gluconic acid, which is then able to protonate the nitrogen-containing compounds as described above to generate a cationic short peptide, with a gluconate counter-anion (denoted (GdL⁻) of the following structure:

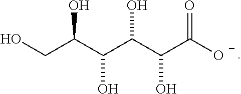 [SMILES: O=C([O-])[C@H](O)[C@@H](O)[C@H](O)[C@H](O)CO]

In some preferred embodiments, A⁻ is trifluoroacetate (TFA⁻). In other preferred embodiments, A⁻ is GdL⁻.

In some embodiments, R¹ is

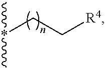

### R⁴ is

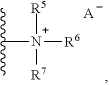

X is hydrogen, and R² and R³, when taken together with the carbon atoms to which they are attached, form a benzene ring. This gives rise to compounds of formula (II):

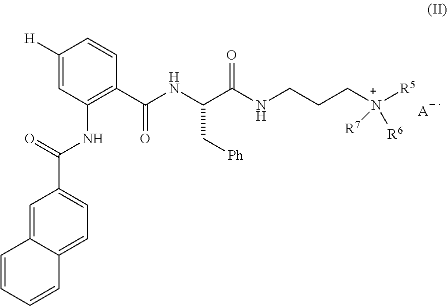

One particularly preferred embodiment is a compound of formula (II) wherein R⁵, R⁶, and R⁷ are H and A⁻ is GdL⁻.

In some embodiments, R¹ is

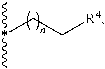

### R⁴ is

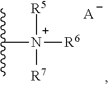

X is fluorine, and R² and R³, when taken together with the carbon atoms to which they are attached, form a benzene ring. This gives rise to compounds of formula (III):

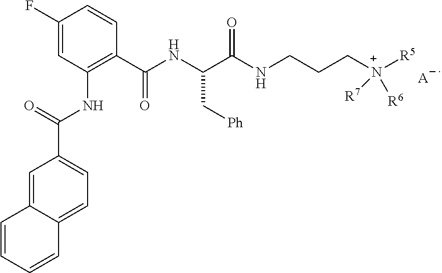

One particularly preferred embodiment is a compound of formula (III) wherein R⁵ is H, R⁶ and R⁷ are methyl, and A⁻ is GdL⁻.

In some embodiments, R¹ is

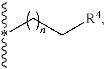

### R⁴ is

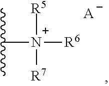

X is chlorine, and R² and R³, when taken together with the carbon atoms to which they are attached, form a benzene ring. This gives rise to compounds of formula (IV):

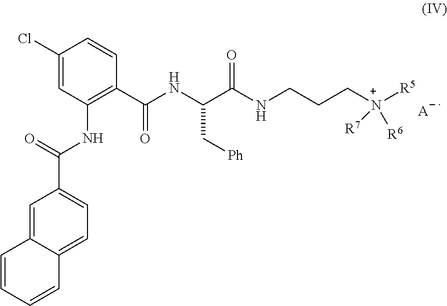

One particularly preferred embodiment is a compound of formula (IV) wherein R⁵ is H, R⁶ and R⁷ are methyl, and A⁻ is GdL⁻.

In the compounds of formulae (II) to (IV), it was surprisingly found that the naphthalene ring was required for good activity and hydrogelation properties. The naphthalene can be substituted with many electron donating or withdrawing groups, whilst still retaining these properties. Examples of electron donating and electron withdrawing groups include, but are not limited to, halo, CN, CF₃, OCF₃, OCH₃, OCH₂CH₃, N(CH₃)₂, N(CH₂CH₃)₂, N(CH₃)(CH₂CH₃), COCH₃, COCH₂CH₃, OCOCH₃, SCH₃, SCH₂CH₃, C₁-C₄ alkyl, C₂-C₄ alkenyl, and C₂-C₄ alkynyl.

In some embodiments, R¹ is

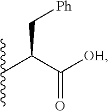 [SMILES: *[C@@H](Cc1ccccc1)C(=O)O]

R² is NO₂, and X is H. This gives rise to compounds of formula (V):

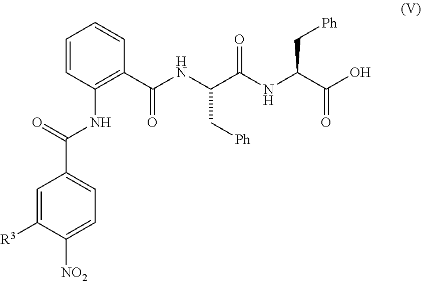

These embodiments can be described as NO-releasing short peptides, as the benzene ring at the N-terminus contains a nitro group, with an adjacent CH₃ or CF₃ group. A particularly preferred embodiment is the compound of formula (V) wherein R³ is CF₃.

These NO-releasing short peptides exert their anti-bacterial activity upon activation by light. In one embodiment, the compound of formula (V) may be activated by ultraviolet light. For example, the compound of formula (V) may be activated by ultraviolet light of wavelength 356 nm. Alternatively, the compound of formula (V) may be activated by irradiation with ultraviolet light selected from UVA, UVB and UVC. UVA is ultraviolet light with a wavelength in the range of 315 nm to 400 nm. UVB is ultraviolet light with a wavelength in the range of 280 nm to 315 nm. UVC is ultraviolet light with a wavelength in the range of 100 nm to 280 nm. In another embodiment, the compound of formula (V) may be activated by blue light, wherein the blue light has a wavelength in the range of 440 nm to 450 nm.

Exemplary compounds according to the present invention include the compounds set forth in the table below:

| | |
|---|---|
| 4 | 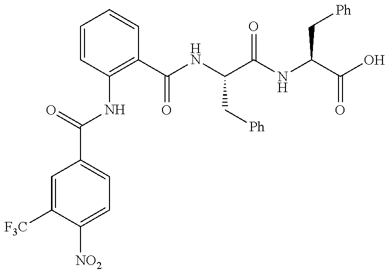 [SMILES: O=C(Nc1ccccc1C(=O)N[C@@H](Cc1ccccc1)C(=O)N[C@@H](Cc1ccccc1)C(=O)O)c1ccc([N+](=O)[O-])c(C(F)(F)F)c1] |
| 10 | 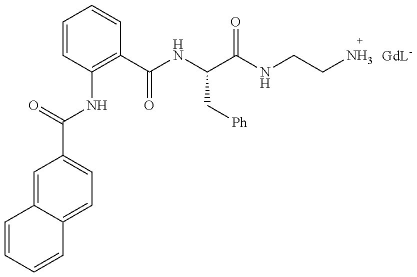 [SMILES: NCCNC(=O)[C@H](Cc1ccccc1)NC(=O)c1ccccc1NC(=O)c1ccc2ccccc2c1] |
| 11 | 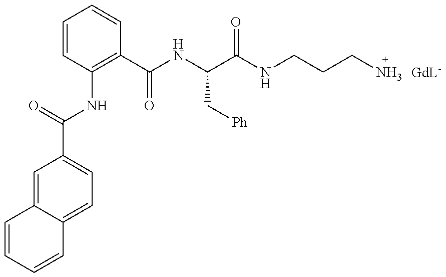 [SMILES: NCCCNC(=O)[C@H](Cc1ccccc1)NC(=O)c1ccccc1NC(=O)c1ccc2ccccc2c1] |
| 12 | 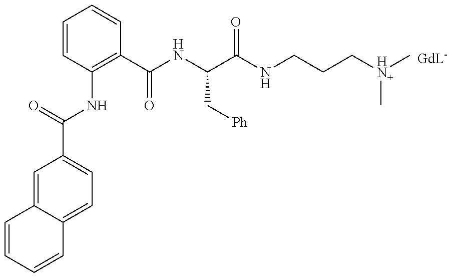 |
| 13 | 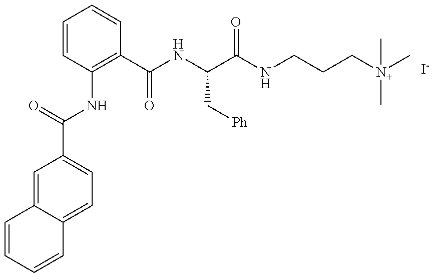 [SMILES: C[N+](C)(C)CCCNC(=O)[C@H](Cc1ccccc1)NC(=O)c1ccccc1NC(=O)c1ccc2ccccc2c1 ; [I-]] |
| 14 | 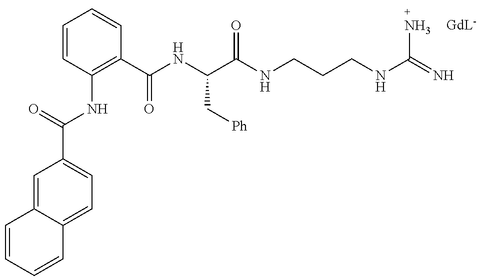 [SMILES: N=C(N)NCCCNC(=O)[C@H](Cc1ccccc1)NC(=O)c1ccccc1NC(=O)c1ccc2ccccc2c1] |
| 15 | 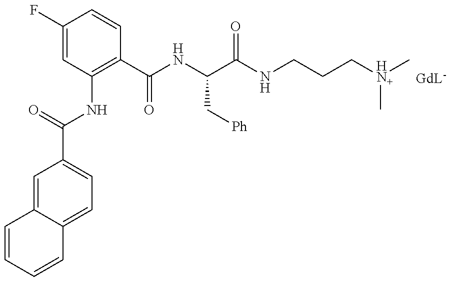 |
| 16 | 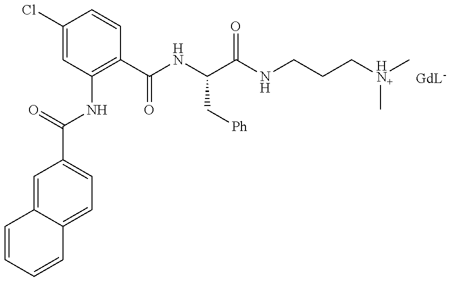 |
| 17 | 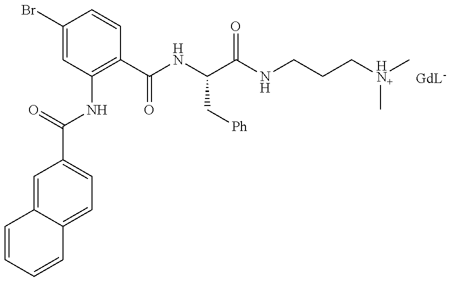 |
| 18 | 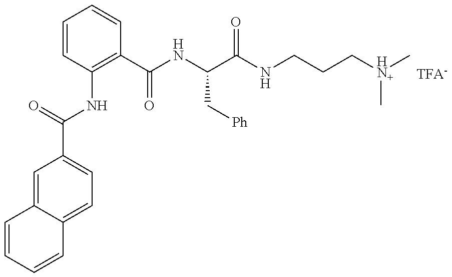 |
| 19 | 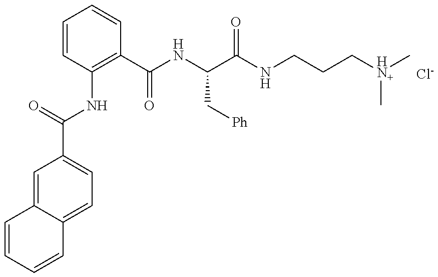 |

In a preferred embodiment the compound is selected from the group consisting of compounds 4, 11, 12, 13, 15, 16, 18 and 19.

In another aspect, the present invention provides an antibacterial hydrogel, comprising water and a compound according to formula (I), as described above. In one embodiment the present invention provides an antibacterial hydrogel comprising water and a compound selected from the group consisting of compounds 4, 11, 12, 13, 15, 16, 18 and 19.

In some embodiments, the antibacterial hydrogel comprising water and a compound of formula (I), wherein R¹ is

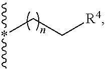

and R² and R³, when taken together with the carbon atoms to which they are attached, form a benzene ring, has a pH of between about 4.0 and about 6.0. Hydrogels which are slightly acidic are suitable for topical applications, as healthy skin surfaces of most human body parts have pH values ranging from 4.1 to 5.8. For example, the hydrogel may have a pH within a range selected from the group consisting of between 4.2 and 5.8, between 4.4 and 5.6, between 4.6 and 5.4, and between 4.8 and 5.2.

In some embodiments, the antibacterial hydrogel comprising water and a compound of formula (I), wherein R¹ is

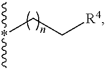

and R² and R³, when taken together with the carbon atoms to which they are attached, form a benzene ring, at a concentration of 1% w/v, exhibits a bacterial reduction of between about 3.0 Log₁₀ and about 9.0 Log₁₀. For example, the bacterial reduction may be within a range selected from the group consisting of from 3.0 Log₁₀ to 4.0 Log₁₀, from 4.0 Log₁₀ to 5.0 Log₁₀, from 5.0 Log₁₀ to 6.0 Log₁₀, from 6.0 Log₁₀ to 7.0 Log₁₀, from 7.0 Log₁₀ to 8.0 Log₁₀, and from 8.0 Log₁₀ to 9.0 Log₁₀. In this embodiment, the bacteria is a Staphylococcus aureus.

In some embodiments, the antibacterial hydrogel comprising water and a compound of formula (I), wherein R¹ is

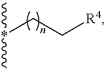

and R² and R³, when taken together with the carbon atoms to which they are attached, form a benzene ring, has a linear viscoelastic region (LVER) of between about 0.98% and about 2.01%. For example, the hydrogel may have a LVER selected from the group consisting of between about 0.98% and about 1.20%, between about 1.20% and about 1.40%, between about 1.40% and about 1.60%, between about 1.60% and about 1.80%, and between about 1.80% and about 2.01%.

In some embodiments, the antibacterial hydrogel comprises water and a compound of formula (I), wherein R¹ is

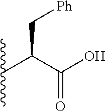 [SMILES: *[C@@H](Cc1ccccc1)C(=O)O]

and R² is NO₂. In these embodiments, the hydrogel has a pH of between about 5.0 and about 8.0. In some embodiments, the hydrogel has a pH within a range selected from the group consisting of between about 5.2 and about 8.0, between about 5.4 and about 7.8, between about 5.6 and about 7.6, between about 5.8 and about 7.4, between about 6.0 and about 7.2, between about 6.2 and about 7.0, and between about 6.4 and about 6.8. In a preferred embodiment, the hydrogel has a pH of about 5.2. In other embodiments, the hydrogel has a pH in a range of between 5.5 and 6.0.

In some embodiments, wherein R¹ is

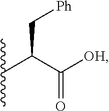 [SMILES: *[C@@H](Cc1ccccc1)C(=O)O]

X is H, R² is NO₂, and R³ is CF₃, the hydrogel may be induced to release NO. In these embodiments, the hydrogel generates NO under UV or blue light irradiation. The concentration of the compound of formula (I) in the hydrogel can be any concentration that is able to form a hydrogel, such as between about 0.1% w/v and about 5% w/v. For example, the concentration of compound of formula (I) may be within a range selected from the group consisting of between about 0.1% w/v and about 4.5% w/v, between about 0.2% w/v and about 4.0% w/v, between about 0.3% w/v and about 3.5% w/v, between about 0.4% w/v and about 3.0% w/v, between about 0.5% w/v and about 2.5% w/v, between about 0.6% w/v and about 2.0% w/v, between about 0.7% w/v and about 1.5% w/v, and between about 0.8% w/v and about 1.0% w/v, The hydrogel, under UV or blue light irradiation, may be irradiated for any amount of time, such as from about 1 second to about 30 minutes. For example, the hydrogel may be irradiated for a period of time selected from the group consisting of between about 10 seconds and about 30 minutes, between about 30 seconds and about 29 minutes, between about 1 minute and about 28 minutes, between about 5 minutes and about 27 minutes, between about 10 minutes and about 26 minutes, between about 15 minutes and about 25 minutes, and between about 20 minutes and about 24 minutes, As one of skill in the art will appreciate, the higher the concentration of compound of formula (I) in the hydrogel, and/or the longer the irradiation time, the greater the concentration of NO will be generated. Thus, the amount of NO generated will be tuneable, based on at least the above variables, which will be advantageous for a range of applications. For example, NO has a minimal bactericidal concentration of about 5 μM, but in some applications lower concentrations of NO may be desirable, such as for biofilm removal. In one embodiment, wherein the compound is at a concentration of 1% w/v, and wherein the hydrogel is irradiated under UV light for 30 minutes, nitric oxide (NO) is generated at a concentration of about 7 μM.

In some embodiments, wherein the antibacterial hydrogel comprises water and a compound of formula (I), wherein R¹ is

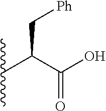 [SMILES: *[C@@H](Cc1ccccc1)C(=O)O]

and R² is NO₂, the hydrogel may have a linear viscoelastic region (LVER) of between about 1.20% and about 1.40%. For example, the LVER may be within a range selected from the group consisting of between 1.10% and 1.45%, between 1.15% and 1.40%, or between 1.20% and 1.35%, or between 1.25% and 1.30%. In one embodiment, wherein the hydrogel is made from a hydrogelator at high pH, the LVER is about 1.20%. In another embodiment, wherein the hydrogel is made from a hydrogelator at a pH of 5-6, the LVER is about 1.40%.

The compounds of the present invention are able to form hydrogels with water at extremely low concentrations of the hydrogelating compound. For example, the minimum amount of compound required to form a hydrogel for compounds of the present invention, the critical gel concentration (CGC), is between about 0.1% w/v and about 0.3% w/v. The CGC may be within a range selected from the group consisting of between about 0.10% w/v and about 0.30% w/v, between about 0.10% w/v and about 0.25% w/v, between about 0.10% w/v and about 0.20% w/v, and between about 0.10% w/v and about 0.15% w/v. A CGC value of 0.1% w/v means that 1.0 mg of a compound is required, in 1.0 mL of water, to form a self-supporting hydrogel.

In some embodiments, the antibacterial hydrogel of the present invention may have a G′/G″ ratio of between about 10 and 18. For example, the G′/G″ ratio may be within a range selected from the group consisting of between about 11 and about 18, between about 12 and about 17, between about 13 and about 16, and between about 14 and about 15.

In some embodiments, the antibacterial hydrogel of the present invention comprises water, a compound of formula (I), and a salt. The salt is not particularly limited, as long as it is able to facilitate formation of a self-supporting hydrogel. For example, the salt may be, but is not limited to, CaCl₂), KBr, KCl, or NaCl, or any combination thereof. In some embodiments, the salt is NaCl (sodium chloride). In other embodiments, the salt may be a combination of NaCl and KCl.

The amount of salt that can be used is not particularly limited, as long as it is able to facilitate formation of a self-supporting hydrogel. In some embodiments, the amount of salt used can be within a range selected from the group consisting of between about 0.01 to about 5.0 equivalents, compared to the hydrogelating compound. For example, the amount of salt used may be within a range selected from the group consisting of between about 0.1 and about 5.0 equivalents, between about 0.5 and about 4.5 equivalents, between about 1.0 and about 3.5 equivalents, between about 1.5 and about 3.0 equivalents, and between about 2.0 and about 2.5 equivalents, compared to the hydrogelating compound,

In another aspect, the present invention provides an antibacterial fiber, comprising a compound according to formula (I) as described herein. Fibers form spontaneously after a compound of the present invention is dissolved in water, even when the concentration of the compound is below the CGC for that compound. The fibers may be characterised as having a fiber diameter of between about 40 nm and about 110 nm.

For example, the fiber diameter may be within a range selected from the group consisting of between about 45 nm and about 105 nm, between about 50 nm and about 100 nm, between about 55 nm and about 95 nm, between about 60 nm and about 90 nm, between about 65 nm and about 85 nm, and between about 70 nm and about 80 nm, In one preferred embodiment, the fiber has a diameter of about 47 nm. In another preferred embodiment, the fiber has a diameter of about 104 nm. In another preferred embodiment, the fiber has a diameter of about 40 nm. In another preferred embodiment, the fiber has a diameter of about 43 nm.

The fibers may form bundles, with a diameter of between about 60 nm and 150 nm. For example, the fiber bundles may have a diameter within a range selected from the group consisting of between about 65 nm and about 145 nm, between about 70 nm and about 140 nm, between about 75 nm and about 135 nm, between about 80 nm and about 130 nm, between about 85 nm and about 125 nm, between about 90 nm and about 120 nm, between about 95 nm and about 115 nm, and between about 100 nm and about 110 nm, In one preferred embodiment, the fiber bundles have a diameter of about 115 nm. In another preferred embodiment, the fiber bundles have a diameter of about 139 nm. In another preferred embodiment, the fiber bundles have a diameter of about 99 nm. In another preferred embodiment, the fiber bundles have a diameter of about 104 nm.

The fibers or fiber bundles may form junction zones, where fibers or fiber bundles cross each other to form a criss-crossing network as shown in the AFM images in the figures. In some embodiments, the fibers do not form bundles. In some embodiments, the fibers or fiber bundles do not form junction zones. In some embodiments, the hydrogels do not form spheroidal aggregates.

In another aspect, the present invention provides for use of the compound, hydrogel, or fiber as described herein, in a barrier material or in an antibacterial carrier material for organ transplantation. A barrier material can be used, for example, on any surface wound where it is desirable to prevent or treat infection from bacteria. The barrier material will provide a physical barrier to the wound site, and also provide a hydrated environment to promote wound healing, as well as antibacterial properties from the compound, hydrogel, or fiber itself. The compound, hydrogel, or fiber can be applied topically, directly to the wound site, or it can be applied to an adhesive bandage or other similar wound dressing, which is then applied to the wound site. If applied topically, the hydrogel can be applied directly, or the compound, hydrogel, or fiber can be formulated into a cream, a lotion, a spray, an ointment, a salve, a gel, a paste, or any such similar medication which is known in the formulation arts.

Alternatively, the compound, hydrogel, or fiber as described herein can be used in an antibacterial carrier material for organ transplantation, such as bone grafts. During organ transplantation an organ is removed from one patient and implanted into another (allografts) or from one area of a patient to another area of the same patient (autografts). The potential for infection is high due to the multiple sources of surface- or air-borne bacteria. As such, it would be beneficial to include in the transplantation process a non-toxic carrier material with antibacterial properties, to improve the chances of success of the transplantation procedure. For example, the carrier material may comprise an immunosuppressant to prevent rejection of the organ, a corticosteroid, or a preservation solution, which has been used to preserve the organ during the time taken to remove it from one location and implant it into the new location. Thus the hydrogel can be used as a carrier material, or the compound, hydrogel, or fiber can be added to carrier material, to provide an antibacterial effect without the addition of a further antibiotic. The carrier material also provides a substrate that can hold any immunosuppressant, corticosteroid, or preservation solution, and would also provide a hydrated environment which could promote healing and prevent bacterial penetration at the implantation site. In some embodiments, the carrier material comprises NO-releasing compounds or fibers, or the carrier material is a NO-releasing hydrogel, wherein the NO is generated by UV or blue light irradiation. Due to the limited penetrative ability of light, it should not affect additives in the bulk carrier material (hydrogel) or the organ.

In another aspect, the present invention provides an antibacterial composition for disinfecting a surface, comprising the compound, hydrogel, or fiber as described herein. The composition can be used alone, or in combination with a cleaning product. The composition can be applied to a surface by any means, such as spraying, dip coating, or painting. The composition could be applied to a tissue or wet-wipe, and applied to the surface by wiping or rubbing. It is not the intention of the inventors to limit the invention by the means by which the composition is applied. In one embodiment, the composition is applied by spraying. In another embodiment, the composition is applied by dip coating.

In another aspect, the present invention provides an antibacterial coating for an article, comprising the compound, hydrogel, or fiber as described herein. The coating may be applied at the time of manufacture of the article, or it may be applied as a treatment, after manufacture. Particularly preferred articles are medical devices and articles which are used in a health care setting, such as surgical equipment, equipment related to surgery, and non-surgical equipment. Surgical equipment includes, but is not limited to, scalpels and scalpel handles, scissors, other cutters, forceps, clamps, retractors, distractors, suction tips and suction tubes, surgical staplers, surgical drills, and calipers. Equipment related to surgery includes, but is not limited to, autoclaves, surgical tables, taps and tap handles, surgical cloths, surgical gowns, and machines such as anesthetic machines, ventilators, and the like. Non-surgical equipment at a health care setting includes, but is not limited to, door handles, light switches, ventilation systems, floors, walls, desks, beds and bedding material, doctor's offices, nurse's offices, and clothing.

In another aspect, the present invention provides a wound dressing for the treatment or prevention of a bacterial infection, comprising the compound, hydrogel, or fiber as described herein. The wound dressing is a sterile substrate which is applied directly to the wound to protect it from further harm and infection. Dressings are usually, but not always, held in place with a bandage or other device. In some embodiments, the wound dressing may have the compound, hydrogel, or fiber impregnated throughout the substrate, or it may be concentrated in a defined area. The compound, hydrogel, or fiber of the present invention may be applied to a layer adjacent to a backing material. In some embodiments, the backing material may include an adhesive substance to allow the dressing to be fixed to the skin around the wound and hold the compound, hydrogel, or fiber in place over the wound.

In another aspect, the present invention provides a method of preventing or treating a bacterial infection, comprising topical administration to a subject a therapeutically effective amount of the compound, hydrogel, or fiber as described herein. In another aspect, the present invention provides a method of treating a wound in a subject in need thereof, comprising administering to the wound an effective amount of a compound, hydrogel, or fiber as described herein. The wound may be a dermal wound, a scratch, a scrape, a cut, an incision, or a burn. The wound may have been caused by surgery. In wounds that are on the skin, the hydrogel may be administered topically. For hydrogels based on cationic short peptides as described herein, activation of the hydrogel is unnecessary. For hydrogels based on NO-releasing short peptides as described herein, activation of the hydrogel is necessary, which may be achieved by irradiation of UV or blue light. In a particular embodiment, the irradiation is performed after the hydrogel has been exposed to the bacteria, to ensure controlled delivery of NO to the bacteria. For example, the hydrogel could be used as a dermal patch which can act as a physical barrier and source of moisture to accelerate wound healing. In addition, upon irradiation, the hydrogel could exert on demand antibacterial properties via NO release.

In some embodiments, the compound, hydrogel, or fiber prevents microbial infection of the wound. In other embodiments, the compound, hydrogel, or fiber treats a wound infected with bacteria. The compound, hydrogel, or fiber can be administered in any form or mode which makes the compound, hydrogel, or fiber effective at treating or preventing pathogens at a wound site. One skilled in the art of preparing formulations can readily select the proper form and mode of administration depending upon the particular characteristics of the compound, hydrogel, or fiber selected, the type of wound to be treated, and other relevant circumstances. We refer the reader to Remington's Pharmaceutical Sciences, 19th edition, Mack Publishing Co. (1995) for further information.

### EXAMPLES

The present invention will now be more fully described by reference to the following non-limiting Examples.

### Example 1—Synthesis

All chemicals and solvents used were purchased from Chem-Impex and Sigma-Aldrich and were used directly without any further purification.

### NO-Releasing Anthranilamide-Based Peptides

To obtain anthranilamide-based short peptides bearing an NO donor, 4-nitro-3-(trifluoromethyl)benzoic acid is reacted with oxalyl chloride to form the corresponding benzoyl chloride 1, as shown below:

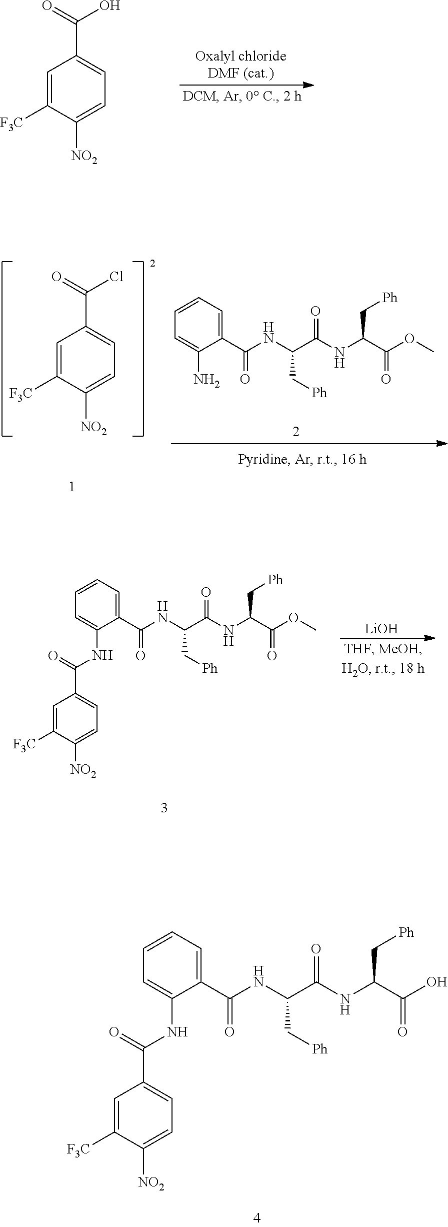

In this step, a catalytic amount of N,N-dimethylformamide (DMF) was employed in the reaction, to generate a reactive iminium intermediate in dichloromethane (DCM), which is then reacted with the carboxylic acid group of the starting material. Intermediate 1 was then reacted immediately with anthranilamide-appended dipeptide 2, at room temperature (r.t.), to provide the methyl-protected, nitrobenzene-appended anthranilamide-based short peptide 3 in 71% yield. Further hydrolysis of compound 3, in tetrahydrofuran (THF), methanol (MeOH) and water, provided the final nitrobenzene-appended short peptide 4 as a yellow powder in quantitative yield.

### Cationic Anthranilamide-Based Peptides

The following describes the synthesis of cationic anthranilamide-based peptides. Four modifications (A-D) were introduced to investigate the effect of alkyl chain length, cationic moiety, halogen substituent, and counter-anion on the hydrogelation and mechanical properties of hydrogels and their biological activities, as summarized below:

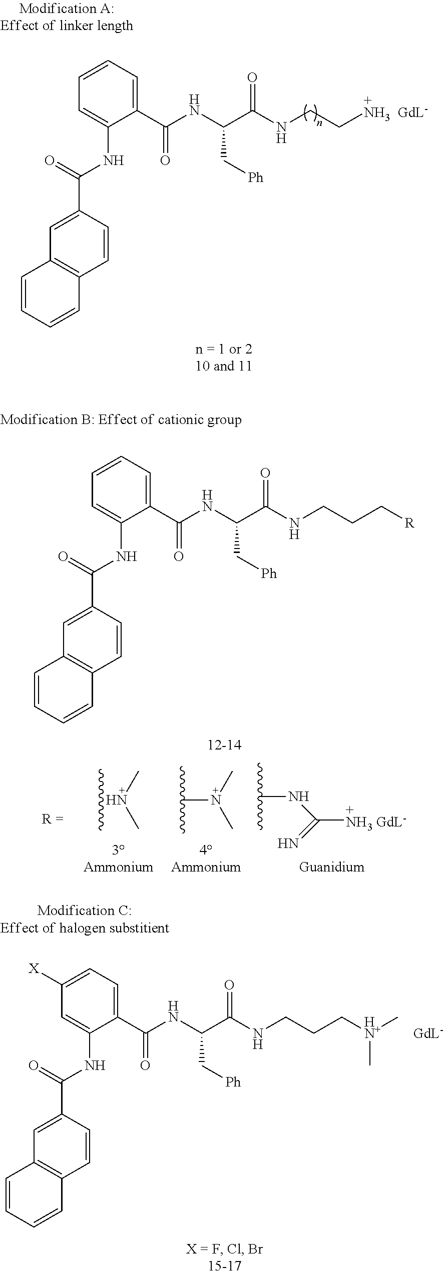

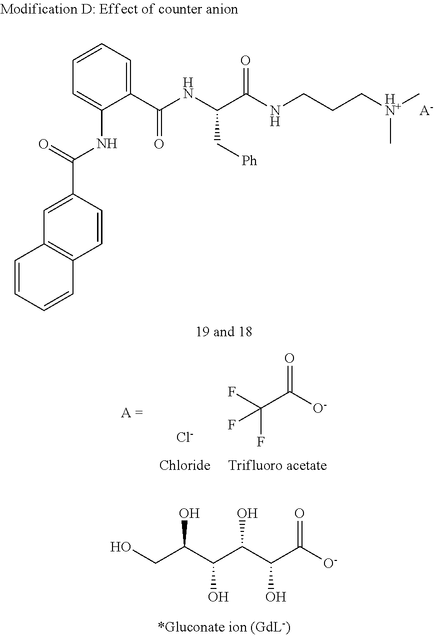

In Modification A, the effect of alkyl chain length in the linker was investigated. A previous study showed that cationic peptide-mimics bearing either two or three aliphatic carbons as the linker displayed antibacterial activity against Gram-positive and Gram-negative bacteria.

The structure of the pendant cationic moiety was varied by incorporating primary, tertiary, or quaternary ammonium groups (Modification B). It has been described that the molecular structure of the cationic moiety in antibacterial peptide derivatives governs their antibacterial potency. In addition, the presence of a guanidinium group in bioactive molecules, including peptides, has been reported to provide selectivity towards bacteria cells. Thus, guanidinium was also explored as an alternative cationic moiety in this modification.

The effect of halogen substituents (i.e. fluoro, chloro, and bromo) on the anthranilamide core was also examined (Modification C). The presence of a halogen in peptides has been associated with enhanced biological activity. In addition, the presence of fluorine on the anthranilamide core of the first generation short peptides did not hinder hydrogel formation.

Lastly, the effect of counter-anion on the properties of the resulting hydrogels and their biological activity was also investigated (Modification D).

### Modification A

The synthesis started with the ring-opening reaction of isatoic anhydride derivatives 5 with methyl ester-protected L-phenylalanine 6, as shown below:

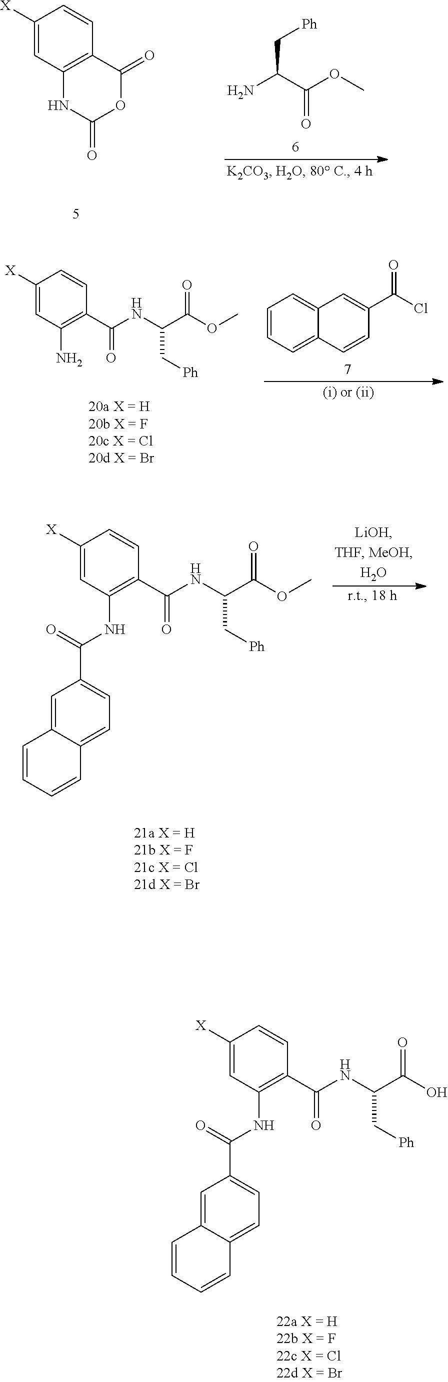

Intermediates 20a-d were obtained as white solids in 65-75% yields. The formation of these intermediates was confirmed using ¹H NMR.

Intermediate 20a was reacted with naphthoyl chloride 7 to obtain the naphthoyl-substituted intermediate 21a in moderate yield (using route (i): Et₃N, DCM, r.t., 18 h, 43%) or excellent yield (using route (ii): Et₃N, 4-dimethylaminopyridine (DMAP), DCM, r.t., 18 h, 90%). Method (ii) was applied to obtain intermediates 21b-d. The methyl ester of intermediates 21a-d was hydrolysed using lithium hydroxide (LiOH) to give the carboxylic acid intermediates 22a-d in quantitative yields.

In the next stage, intermediate 22a was subjected to an amide coupling reaction with various Boc-protected alkyl diamines (8a, n=1; 8b, n=2), as shown below:

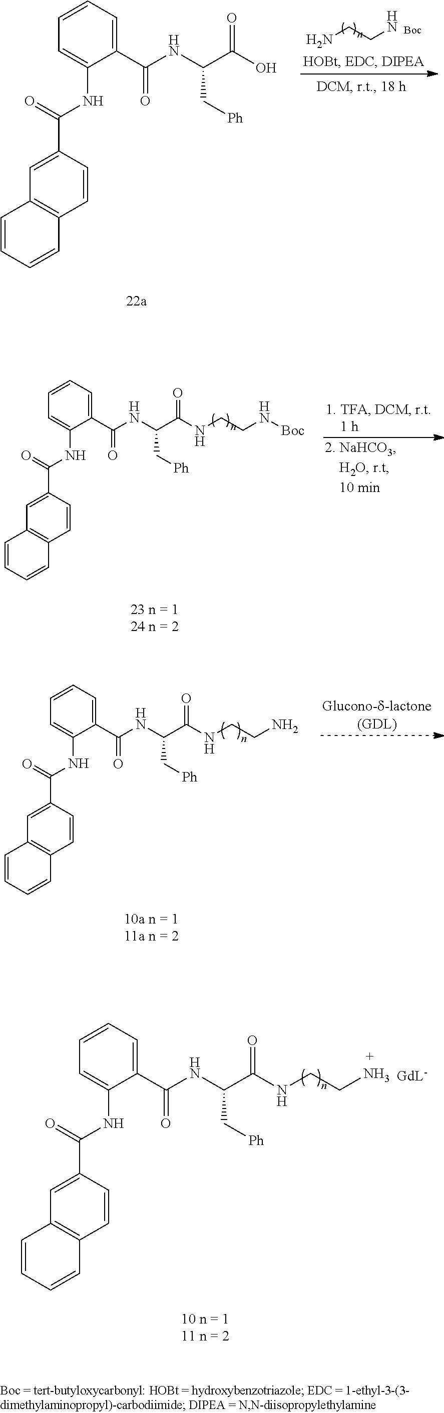

Intermediates 23 and 24 were obtained as pure white solids in 54-50% yields. Afterwards, the Boc-protecting group was removed with trifluoroacetic acid (TFA). A subsequent NaHCO₃ wash of the resulting solids provided the free amines 10a and 11a in quantitative yields. The addition of glucono-S-lactone (GdL), to induce hydrogel formation, provided the short cationic peptides 10 and 11.

### Modifications B and C

The synthesis of anthranilamide-based short cationic peptides bearing tertiary and quaternary cationic groups is outlined below:

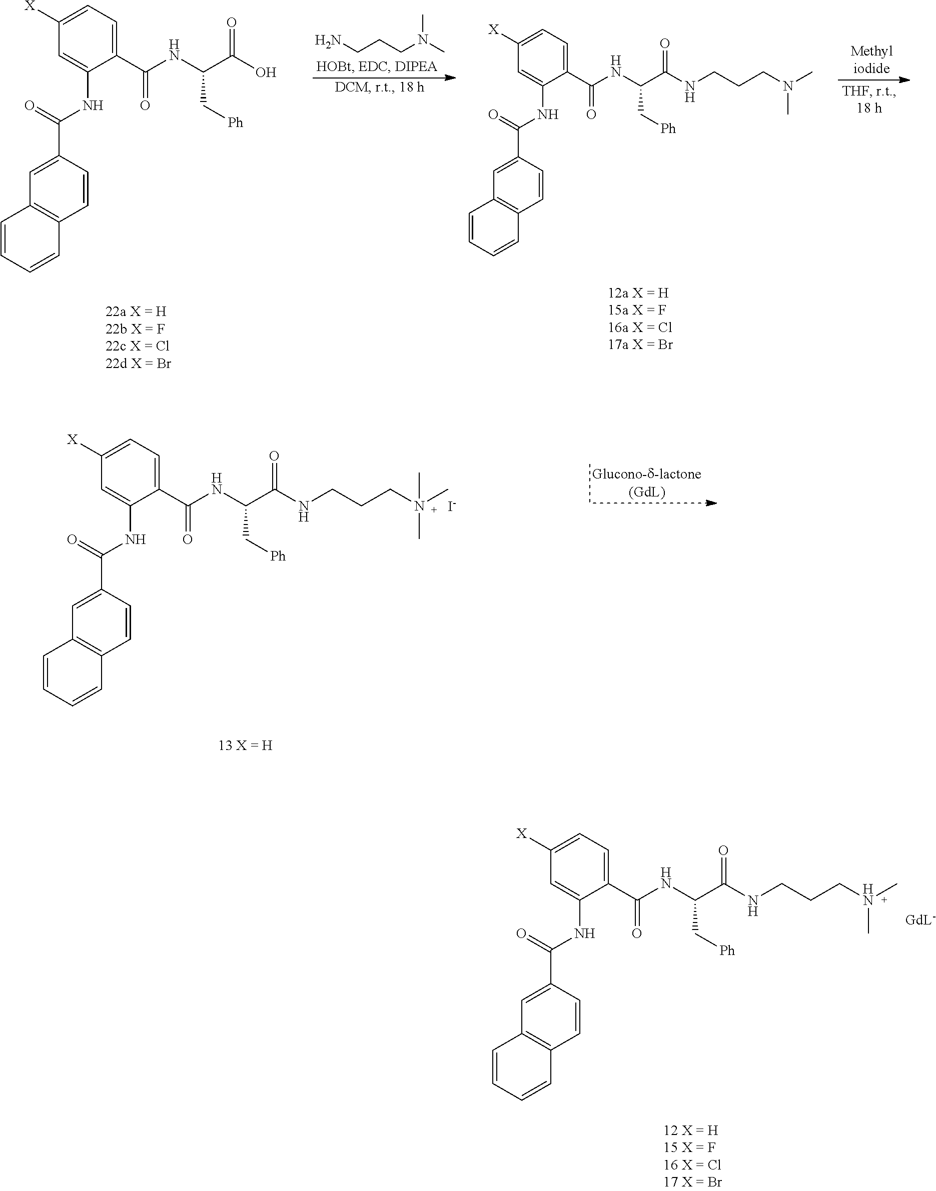

Amide coupling reactions between carboxylic acids 22a-d and N,N-dimethylpropane-1,3-diamine (8c) gave peptides 12a and 15a-17a in 70-78% yield. The addition of GdL during hydrogel formation will provide the tertiary ammonium short peptides 12 and 15-17. Meanwhile, the quaternary ammonium group was introduced by treating a suspension of 12a in THF with methyl iodide to provide the short cationic peptide 13 in 95% yield.

A guanidinium group was introduced by treating the primary amine 11a with N,N′-di-Boc-1H-pyrazole-1-carboxamidine 9 in the presence of Et₃N, giving intermediate 25:

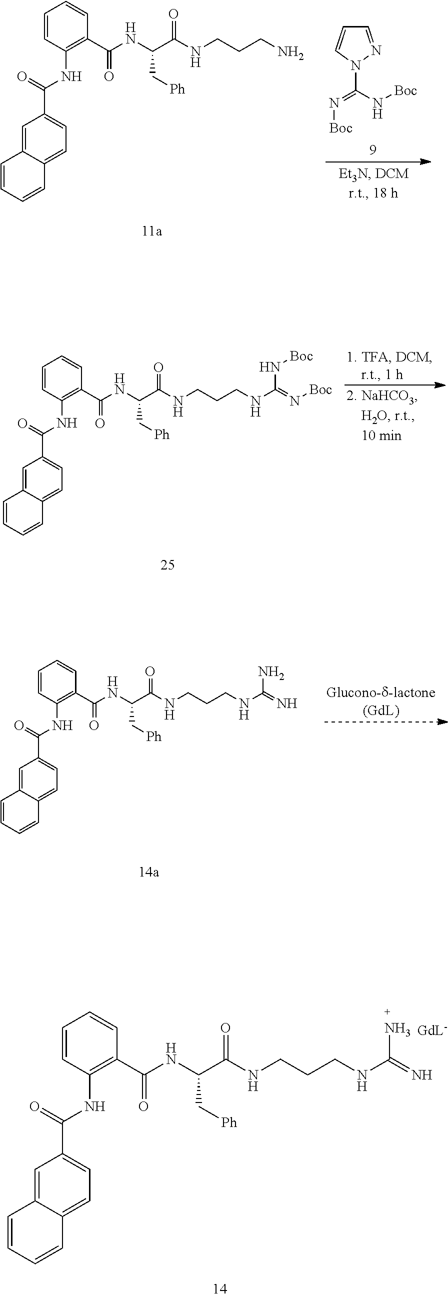 [SMILES: NCCCNC(=O)[C@H](Cc1ccccc1)NC(=O)c1ccccc1NC(=O)c1ccc2ccccc2c1 ; CC(C)(C)OC(=O)/N=C(\NC(=O)OC(C)(C)C)n1cccn1 ; CC(C)(C)OC(=O)/N=C(/NCCCNC(=O)[C@H](Cc1ccccc1)NC(=O)c1ccccc1NC(=O)c1ccc2ccccc2c1)NC(=O)OC(C)(C)C ; N=C(N)NCCCNC(=O)[C@H](Cc1ccccc1)NC(=O)c1ccccc1NC(=O)c1ccc2ccccc2c1 ; N=C(N)NCCCNC(=O)[C@H](Cc1ccccc1)NC(=O)c1ccccc1NC(=O)c1ccc2ccccc2c1]

The Boc group was removed using TFA, and was neutralized with NaHCO₃ to afford the guanidine compound 14a in quantitative yield. The addition of GdL during hydrogel formation will provide the guanidinium compound 14.

### Modification D

To vary the counter-anion, compound 12a was reacted with either (i) TFA or (ii) 4M HCl to provide short cationic peptides 18 and 19 respectively:

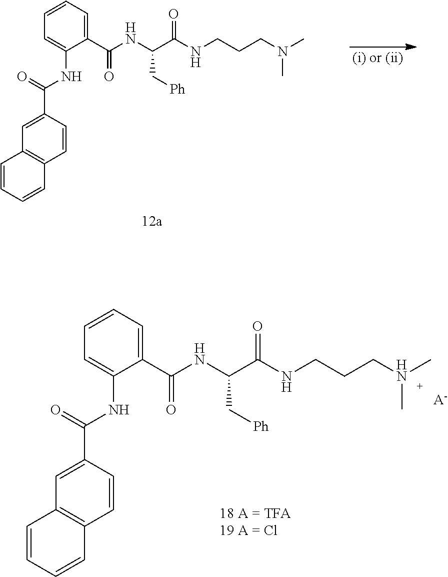

### General Procedures

### A. Ring-Opening of Isatoic Anhydride Derivatives

L-Phenylalanyl hydrochloride salt (F.HCl) (1.0 equivalent) was dissolved in Milli-Q water. Potassium carbonate (2.0 equivalents) was added and the reaction mixture was stirred at room temperature for 20 minutes. After the reaction mixture turned clear, isatoic anhydride derivatives (1.0 equivalent), dissolved in acetone, were added and allowed to react at room temperature for another 18 hours. After completion, the reaction mixture was removed under reduced pressure to remove the acetone. The resulting precipitate was then filtered and dried to provide intermediates 20a-d as white solids in 65-73% yield.

### B. Naphthoylation

The ring-opened isatoic anhydride derivatives 20a-d were suspended in anhydrous dichloromethane (1-5 mL) under argon atmosphere, followed by addition of Et₃N (2.0 equivalents) and DMAP (0.1 equivalents). After stirring at room temperature for 10 minutes, the reaction mixture was cooled to 0° C. and naphthoyl chloride (1.5 equivalents) was added slowly. Afterwards, the reaction mixture was warmed to room temperature and stirred for 4-6 hours. After completion, the reaction mixture was removed under reduced pressure. The resulting crude material was purified using column chromatography with hexane:ethyl acetate to afford 21a-d as white solids in 80-90% yield.

### C. Hydrolysis

The methyl ester-protected intermediates 21a-d were dissolved in THF:MeOH:H₂O with volume ratio of 10:5:2. LiOH (3.0 equivalents) was added to the reaction mixture which was then stirred for 18 hours. The reaction mixture was diluted with Milli-Q water (20 mL) and washed with diethyl ether (3×15 mL). The aqueous phase was acidified until pH 3-4 and was extracted with ethyl acetate (2×25 mL). The resulting organic phase was dried over sodium sulfate and concentrated under reduced pressure to afford 22a-d as white solids in quantitative yield.

### D. Amide Coupling

Compounds 22a-d, bearing a carboxylic acid end (1.0 equivalent), were dissolved in anhydrous DMF under nitrogen atmosphere followed by addition of HOBt (1.2 equivalents). The reaction mixture was cooled to 0° C. and stirred for 30 minutes. Boc-protected diamines or N,N-dimethylpropane-1,3-diamine (1.1 equivalents) in DMF were then added dropwise to the cooled reaction mixture. Subsequently, EDC.HCl (1.2 equivalents) was added followed by the addition of DIPEA (2.0 equivalents). The cloudy reaction mixture was warmed to room temperature and stirred for 18 hours. After completion, the reaction mixture was poured into an ice-water mixture, filtered, and dried. The resulting crude solids were purified by column chromatography to give the pure solids in 50-78% yields.

### E. Boc-Deprotection

Boc-protected compounds were dissolved in anhydrous DCM under nitrogen atmosphere. The clear solution was cooled to 0° C. for 10 minutes followed by addition of either TFA or HCl (4M in dioxane) (2.0 equivalents). The reaction mixture was allowed to warm to room temperature and stirred for 2-18 hours. After completion, indicated by thin layer chromatography (TLC), the reaction mixture was concentrated under reduced pressure to remove excess solvent and acid. The resulting crude material was washed by diethyl ether, filtered, and dried to provide the products in quantitative yield.

### Synthetic Procedures and Characterization Data

### 4-Nitro-3-(trifluoromethyl)benzoyl Chloride 1

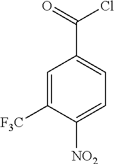 [SMILES: O=C(Cl)c1ccc([N+](=O)[O-])c(C(F)(F)F)c1]

4-Nitro-3-(trifluoromethyl)benzoic acid (0.93 g, 3.9 mmol) was suspended in anhydrous DCM under argon atmosphere. After cooling the suspension to 0° C., oxalyl chloride (4.1 mmol) was added, followed by addition of a couple of drops of anhydrous DMF. The reaction was left to stir at 0° C. for 2 hours. After the reaction was complete, as monitored by TLC, the solvent was removed under reduced pressure. The 4-nitro-3-(trifluoromethyl)benzoyl chloride, formed as a pale yellow intermediate, was immediately used for further reaction without purification. During the reaction and work-up, exposure to light was minimized by using aluminium foil.

### Methyl (2-aminobenzoyl)-L-phenylalanyl-L-phenylalaninate 2

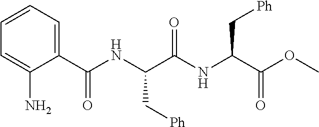 [SMILES: COC(=O)[C@H](Cc1ccccc1)NC(=O)[C@H](Cc1ccccc1)NC(=O)c1ccccc1N]

Compound 2 was prepared by General Procedure A using isatoic anhydride (0.86 g, 5.28 mmol) and methyl-L-phenylalanyl-L-phenylalaninate hydrochloride salt (FF.HCl) (5.80 mmol). After purification by column chromatography using hexane:ethyl acetate (60:40) a fluffy white product was obtained (85% yield, 1.9 g). ¹H NMR (DMSO-d₆, 400 MHz) 8.45 (1H, d, J=8 Hz, ArH), 8.18 (1H, d, 8.42, NH), 7.42 (1H, d, J=8.0 Hz, ArH), 7.31 (2H, d, J=7.1 Hz, ArH), 7.22-7.27 (6H, m, ArH), 7.09-7.22 (3H, m, ArH), 6.63 (1H, d, J=8.3 Hz, ArH), 6.47-6.49 (1H, m, ArH), 6.28 (2H, s, NH₂), 4.64-4.70 (1H, m, CH), 4.49-4.54 (1H, m, CH), 3.59 (1H, s, OCH₃), 3.04-3.09 (2H, m, CH₂), 2.90-3.04 (2H, m, CH₂).

### Methyl (2-(4-nitro-3-(trifluoromethyl)benzamido)benzoyl)-L-phenylalanyl-L-phenylalaninate 3

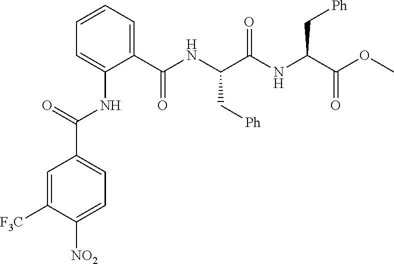 [SMILES: COC(=O)[C@H](Cc1ccccc1)NC(=O)[C@H](Cc1ccccc1)NC(=O)c1ccccc1NC(=O)c1ccc([N+](=O)[O-])c(C(F)(F)F)c1]

Dipeptide-appended anthranilamide 2 (1.8 g, 3.9 mmol) was dissolved in anhydrous pyridine under an argon atmosphere and stirred for 10 minutes. After the reaction mixture was cooled to 0° C., a solution of the freshly made 4-nitro-3-(trifluoromethyl)benzoyl chloride 1 (1.0 g, 3.9 mmol) in anhydrous DMF was added slowly. The reaction mixture was left to stir at room temperature for another 16 hours. After the reaction was complete, the mixture was poured into ice-water and the resulting yellow precipitate was filtered and dried. The crude product was then subjected to column chromatography using hexane:ethyl acetate (60:40) and gave a bright yellow solid as the desired product (71% yield, 1.6 g). During the reaction, work-up, and purification, exposure to light was minimized by using aluminum foil. IR (cm⁻¹): 3298, 2930, 1738, 1654, 1529, 1523, 1446, 1289, 1239, 1196, 1148, 1027, 860, 921, 751, 701. ¹H NMR (400 MHz, DMSO-d₆) 12.14 (1H, s, NH), 9.00 (1H, d, J=8.5 Hz, NH), 8.59 (1H, d, J=7.5 Hz, ArH), 8.40 (1H, dd, J=8.3, 1.2 Hz, NH), 8.35-8.27 (2H, m, ArH), 8.23 (1H, dd, J=8.4, 1.9 Hz, ArH), 7.74 (1H, dd, J=7.9, 1.5 Hz, ArH), 7.58 (1H, ddd, J=8.5, 7.4, 1.5 Hz, ArH), 7.36-7.30 (2H, m, ArH), 7.30-7.22 (1H, m, ArH), 7.22-7.07 (7H, m, ArH), 7.08-6.99 (1H, m, ArH), 4.80 (1H, ddd, J=11.0, 8.5, 4.0 Hz, CH), 4.53 (1H, ddd, J=8.5, 7.5, 5.8 Hz, CH), 3.56 (3H, s, OCH₃), 3.09 (2H, ddd, J=27.2, 13.8, 4.8 Hz, CH₂), 3.02-2.86 (2H, m, CH₂). ¹³C NMR (101 MHz, DMSO-d₆) 171.68, 170.95, 168.27, 161.55, 138.66, 138.06, 137.96, 136.98, 132.67, 132.29, 129.14, 128.99, 128.57, 128.14, 127.96, 126.92, 126.47, 126.21, 126.17, 123.85, 121.81, 121.02, 54.42, 53.68, 51.82, 36.82, 36.43. HR-MS (ESI): calcd for C₃₄H₂₉F₃N₄O₇Na: 685.1886, found 685.1877.

### (2-(4-Nitro-3-(trifluoromethyl)benzamido)benzoyl)-L-phenylalanyl-L-phenylalanine 4

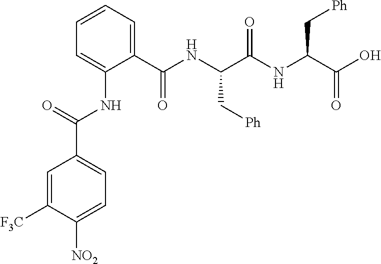 [SMILES: O=C(Nc1ccccc1C(=O)N[C@@H](Cc1ccccc1)C(=O)N[C@@H](Cc1ccccc1)C(=O)O)c1ccc([N+](=O)[O-])c(C(F)(F)F)c1]

Compound 4 was prepared by General Procedure C using 3 (1.0 g, 1.7 mmol) to give hydrogelator 4 as a yellow powder in quantitative yield (0.97 g). IR (cm⁻¹): 3365, 3268, 3064, 2927, 2553, 2321, 1722, 1640, 1599, 1517, 1445, 1362, 1283, 1237, 1135, 1047, 1001, 920, 846, 699. ¹H NMR (400 MHz, DMSO-d₆) 12.80 (1H, bs, OH), 12.15 (1H, s, NH), 8.99 (1H, d, J=8.6 Hz, NH), 8.42 (2H, dt, J=7.9, 1.3 Hz, ArH), 8.35-8.27 (2H, m, ArH), 8.19 (1H, dd, J=8.4, 1.9 Hz, NH), 7.74 (1H, dd, J=7.9, 1.6 Hz, ArH), 7.59 (1H, ddd, J=8.6, 7.4, 1.5 Hz, ArH), 7.35-7.22 (3H, m, ArH), 7.22-6.99 (8H, m, ArH), 4.79 (1H, ddd, J=11.1, 8.6, 3.8 Hz, CH), 4.48 (1H, td, J=8.2, 5.2 Hz, CH), 3.19-3.02 (2H, m, CH₂), 2.94 (2H, ddd, J=13.7, 9.8, 6.6 Hz, CH₂). ¹³C NMR (101 MHz, DMSO-d₆) 172.65, 170.76, 168.27, 161.49, 138.63, 138.13, 138.07, 137.37, 132.59, 132.34, 129.16, 129.06, 128.57, 128.04, 127.94, 126.96, 126.30, 126.21, 126.15, 123.79, 121.53, 120.90, 54.50, 53.52, 36.81, 36.47. HRMS (ESI): calcd for C₃₃H₂₇F₃N₄O₇Na: 671.1730, found 671.1719.

### Methyl (2-aminobenzoyl)-L-phenylalaninate 20a

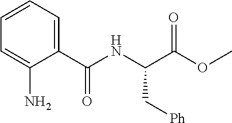 [SMILES: COC(=O)[C@H](Cc1ccccc1)NC(=O)c1ccccc1N]

Compound 20a was prepared by General Procedure A, using F.HCl (4.0 g, 19.0 mmol) and isatoic anhydride (1.0 equivalent). Compound 20a was obtained as a white solid (3.7 g, 65% yield). ¹H NMR (400 MHz, DMSO-d₆) 8.62 (1H, d, J=7.6 Hz, NH), 7.56 (1H, d, J=7.8 Hz ArH), 7.36-7.41 (4H, m, ArH), 7.28-7.31 (1H, m, ArH), 7.21-7.25 (1H, m, ArH), 6.76 (1H, d, J=8.4 Hz, ArH), 6.59 (1H, t, J=7.8 Hz, ArH), 6.42 (2H, bs, NH₂), 4.67-4.73 (1H, m, CH), 3.73 (3H, s, OCH₃), 3.15-3.26 (2H, m, CH₂).

### Methyl (2-amino-4-fluorobenzoyl)-L-phenylalaninate 20b

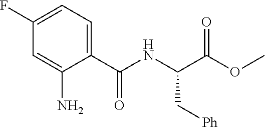 [SMILES: COC(=O)[C@H](Cc1ccccc1)NC(=O)c1ccc(F)cc1N]

Compound 20b was prepared by General Procedure A using F.HCl (1.0 g, 4.6 mmol) and 5-fluoroisatoic anhydride (1.0 equivalent) to afford 20b as a white solid (1.20 g, 82% yield). IR (cm⁻¹): 3463, 3370, 3325, 3022, 2958, 2111, 1745, 1631, 1594, 1511, 1440, 1371, 1296, 1223, 1174, 986, 946, 880, 812, 756, 697. ¹H NMR (400 MHz, DMSO-d₆) 8.61 (d, 1H, J=7.7 Hz, NH), 7.37-7.25 (m, 5H, ArH), 7.25-7.14 (m, 1H, ArH), 7.05 (ddd, 1H, J=9.0, 8.1, 3.0 Hz, ArH), 6.68 (dd, 1H, J=9.0, 5.0 Hz, ArH), 6.23 (s, 2H, NH₂), 4.60 (ddd, 1H, J=10.1, 7.6, 5.3 Hz, CH), 3.63 (s, 3H, OCH₃), 3.19-3.01 (m, 2H, CH₂). ¹³C NMR (101 MHz, DMSO-d₆) 172.24, 167.93, 151.34, 146.58, 137.73, 129.02, 128.25, 126.50, 119.71, 119.48, 117.65, 117.58, 113.75, 113.53, 113.16, 113.10, 53.99, 51.94, 36.11. HRMS (ESI): calcd for C₁₇H₁₇FN₂O₃+Na: 339.1121 found 339.1128.

### Methyl (2-amino-4-chlorobenzoyl)-L-phenylalaninate 20c

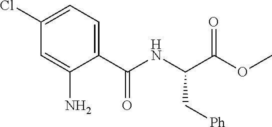 [SMILES: COC(=O)[C@H](Cc1ccccc1)NC(=O)c1ccc(Cl)cc1N]

Compound 20c was prepared by General Procedure A using F.HCl (1.0 g, 5.1 mmol) and 5-chloroisatoic anhydride (1.0 equivalent) to give 20c as a white solid (1.48 g, 87% yield). IR (cm⁻¹): 3448, 3353, 3316 3030, 2955, 2658, 2116, 1741, 1632, 1585, 1519, 1441, 1371, 1303, 1258, 1217, 1173, 1107, 981, 818, 758, 699. ¹H NMR (400 MHz, DMSO-d₆) 8.70 (1H, d, J=7.7 Hz, NH), 7.54 (1H, d, J=2.5 Hz, ArH), 7.28 (4H, d, J=4.3 Hz, ArH), 7.24-7.12 (2H, m, ArH), 6.69 (1H, d, J=8.8 Hz, ArH), 6.47 (2H, s, NH), 4.60 (1H, ddd, J=10.0, 7.7, 5.4 Hz, CH), 3.63 (3H, s, OCH₃), 3.20-3.01 (2H, m, CH₂). ¹³C NMR (101 MHz, DMSO-d₆) 172.24, 167.78, 148.71, 137.73, 131.81, 129.01, 128.26, 127.56, 126.51, 118.06, 117.61, 114.30, 53.96, 51.94, 36.09. HRMS (ESI): calcd for C₁₇H₁₇ClN₂O₃+Na: 355.0825 found 355.0820.

### Methyl (2-amino-4-bromobenzoyl)-L-phenylalaninate 20d

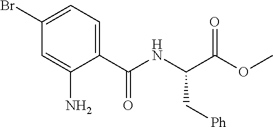 [SMILES: COC(=O)[C@H](Cc1ccccc1)NC(=O)c1ccc(Br)cc1N]

Compound 20d was prepared by General Procedure A using F.HCl (1.0 g, 4.1 mmol) and 5-bromoisatoic anhydride (1.0 equivalent) to afford 20d as an off-white solid (1.3 g, 80% yield). IR (cm⁻¹): 3447, 3353, 3313, 3026, 2951, 1741, 1616, 1582, 1438, 1368, 1303, 1259, 1216, 1163, 1097, 979, 816, 757, 698. ¹H NMR (300 MHz, DMSO-d₆) 8.71 (1H, d, J=7.7 Hz, NH), 7.66 (1H, d, J=2.4 Hz, ArH), 7.28 (4H, d, J=4.4 Hz, ArH), 7.26-7.17 (2H, m, ArH), 6.65 (1H, d, J=8.8 Hz, ArH), 6.49 (2H, s, NH₂), 4.60 (1H, ddd, J=9.8, 7.6, 5.6 Hz, CH), 3.63 (3H, s, OCH₃), 3.21-2.96 (2H, m, CH₂). ¹³C NMR (101 MHz, DMSO-d₆) 172.23, 167.68, 149.02, 137.72, 134.48, 130.38, 129.00, 128.24, 126.50, 118.45, 114.94, 104.78, 53.95, 51.93, 36.09. HRMS (ESI): calcd for C₁₇H₁₇ClN₂O₃+Na: 399.0320 found 399.0327.

### Methyl (2-(2-naphthamido)benzoyl)-L-phenylalaninate 21a

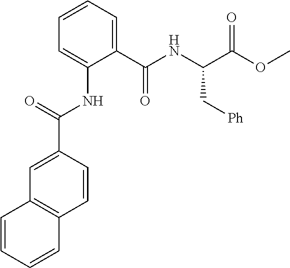 [SMILES: COC(=O)[C@H](Cc1ccccc1)NC(=O)c1ccccc1NC(=O)c1ccc2ccccc2c1]

Compound 21a was prepared by General Procedure B using compound 20a (3.0 g, 10 mmol) to provide compound 21a as a white fluffy solid (4.0 g, 90% yield). IR (cm⁻¹): 3364, 2953, 1743, 1676, 1598, 1520, 1441, 1368, 1289, 1215, 1117, 1093, 980,914, 858, 756, 716. ¹H NMR (400 MHz, DMSO-d₆) 12.08 (1H, s, NH), 9.28 (1H, d, J=7.9 Hz, NH), 8.62 (1H, dd, J=8.4, 1.2 Hz, ArH), 8.46 (1H, d, J=1.8 Hz, ArH), 8.14-8.06 (2H, m, ArH), 8.06-8.00 (1H, m, ArH), 7.89 (1H, dd, J=8.6, 1.9 Hz, ArH), 7.78 (1H, dd, J=7.9, 1.5 Hz, ArH), 7.71-7.63 (2H, m, ArH), 7.63-7.57 (1H, m, ArH), 7.35-7.27 (2H, m, ArH), 7.27-7.19 (3H, m, ArH), 7.15-7.06 (1H, m, ArH), 4.81 (1H, ddd, J=10.4, 7.8, 5.0 Hz, CH), 3.65 (3H, s, OCH₃), 3.28-3.03 (2H, m, CH₂). ¹³C NMR (101 MHz, DMSO-d₆) 172.69, 168.77, 164.45, 139.14, 137.95, 134.43, 132.44, 132.21, 131.81, 129.12, 129.05, 128.69, 128.35, 128.14, 127.77, 127.70, 127.08, 126.38, 123.21, 122.88, 120.34, 120.22, 54.12, 51.90, 36.17. HRMS (ESI): calcd for C₂₈H₂₄N₂O₄+Na: 475.1632 found 475.1626

### Methyl (2-(2-naphthamido)-4-fluorobenzoyl)-L-phenylalaninate 21b

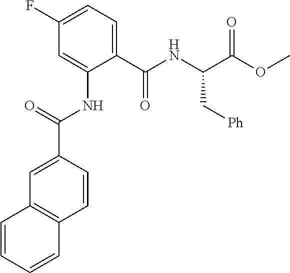 [SMILES: COC(=O)[C@H](Cc1ccccc1)NC(=O)c1ccc(F)cc1NC(=O)c1ccc2ccccc2c1]

Compound 21b was prepared by General Procedure B using compound 20b (1.0 g, 3.1 mmol) to give 21b as a pure white fluffy solid (1.2 g, 82% yield). IR (cm⁻¹): 3328, 3031, 2952, 2108, 1898, 1743, 1671, 1601, 1517, 1445, 1409, 1297, 1251, 1198, 1167, 960, 867, 814, 753, 700. ¹H NMR (400 MHz, DMSO-d₆) 12.08 (1H, s, NH), 9.33 (1H, d, J=7.9 Hz, NH), 8.74 (1H, dd, J=9.2, 5.3 Hz, ArH), 8.56-8.39 (1H, m, ArH), 8.14-8.06 (2H, m, ArH), 8.03 (1H, dd, J=7.9, 1.7 Hz, ArH), 7.93 (1H, dd, J=8.6, 1.9 Hz, ArH), 7.72-7.61 (3H, m, ArH), 7.50-7.40 (1H, m, ArH), 7.38-7.31 (2H, m, ArH), 7.30-7.19 (2H, m, ArH), 7.16-7.06 (1H, m, ArH), 4.87 (1H, ddd, J=10.3, 7.7, 5.0 Hz, CH), 3.69 (3H, s, OCH₃), 3.28-3.09 (2H, m, CH₂). ¹³C NMR (101 MHz, DMSO-d₆) 171.56, 167.79, 164.46, 137.52, 134.69, 132.45, 131.88, 129.11, 129.05, 128.63, 128.19, 128.05, 127.81, 127.73, 127.02, 126.52, 123.28, 122.60, 122.53, 121.60, 119.21, 118.99, 115.06, 114.81, 54.31, 51.87, 36.36. HRMS (ESI): calcd for C₂₈H₂₃FN₂O₄+Na: 493.1540 found 493.1543.

### Methyl (2-(2-naphthamido)-4-chlorobenzoyl)-L-phenylalaninate 21c

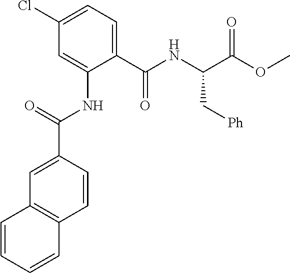 [SMILES: COC(=O)[C@H](Cc1ccccc1)NC(=O)c1ccc(Cl)cc1NC(=O)c1ccc2ccccc2c1]

Compound 21c was prepared by General Procedure B using compound 20c (1.0 g, 3 mmol) to afford compound 21c as an off-white fluffy solid (1.3 g, 87% yield). IR (cm⁻¹): 3344, 2943, 2111, 1732, 1680, 1589, 1517, 1444, 1396, 1347, 1298, 1219, 11171, 1107, 1171, 1025, 936, 807, 752, 703. ¹H NMR (300 MHz, DMSO-d₆) 11.97 (1H, s, NH), 9.41 (1H, d, J=7.8 Hz, NH), 8.63 (1H, d, J=9.0 Hz, ArH), 8.45 (1H, d, J=1.8 Hz, ArH), 8.16-7.97 (3H, m, ArH), 7.92-7.77 (2H, m, ArH), 7.73-7.57 (3H, m, ArH), 7.35-7.26 (2H, m, ArH), 7.22 (2H, t, J=7.6 Hz, ArH), 7.11 (1H, d, J=7.3 Hz, ArH), 4.81 (1H, ddd, J=10.2, 7.8, 5.2 Hz, CH), 3.65 (3H, s, OCH₃), 3.27-2.99 (2H, m, CH₂). ¹³C NMR (101 MHz, DMSO-d₆) 171.56, 167.79, 164.46, 137.52, 134.69, 132.45, 131.88, 129.11, 129.05, 128.63, 128.19, 128.05, 127.81, 127.73, 127.02, 126.52, 123.28, 122.60, 122.53, 121.60, 119.21, 118.99, 115.06, 114.81, 54.31, 51.87, 36.36. HRMS (ESI): calcd for C₂₈H₂₃ClN₂O₄+Na: 509.1244 found 5091240.

### Methyl (2-(2-naphthamido)-4-bromobenzoyl)-L-phenylalaninate 21d

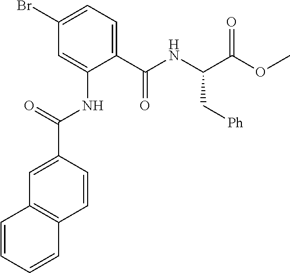 [SMILES: COC(=O)[C@H](Cc1ccccc1)NC(=O)c1ccc(Br)cc1NC(=O)c1ccc2ccccc2c1]

Compound 21d was prepared by General Procedure B using compound 20d (1 g, 2.7 mmol) to provide 21d as a brownish white solid (1.1 g, 80% yield). IR (cm⁻¹): 3326, 2943, 1733, 1684, 1587, 1505, 1453, 1392, 1349, 1299, 1220, 1200, 1093, 1025, 913, 827, 751, 702. ¹H NMR (300 MHz, DMSO-d₆) 11.97 (1H, s, NH), 9.42 (1H, d, J=7.8 Hz, NH), 8.57 (1H, d, J=8.9 Hz, ArH), 8.45 (1H, s, ArH), 8.10 (2H, dd, J=9.0, 4.3 Hz, ArH), 8.03 (1H, d, J=7.8 Hz, ArH), 7.95 (1H, d, J=2.4 Hz, ArH), 7.91-7.83 (1H, m, ArH), 7.80 (1H, dd, J=8.9, 2.3 Hz, ArH), 7.66 (2H, ddd, J=6.7, 4.0, 1.8 Hz, ArH), 7.29 (2H, d, J=7.5 Hz, ArH), 7.22 (2H, t, J=7.5 Hz, ArH), 7.11 (1H, d, J=7.2 Hz, ArH), 4.81 (1H, ddd, J=10.1, 7.7, 5.3 Hz, CH), 3.65 (3H, s, OCH₃), 3.27-3.01 (2H, m, CH₂). ¹³C NMR (101 MHz, DMSO-d₆). 171.56, 167.79, 164.46, 137.52, 134.69, 132.45, 131.88, 129.11, 129.05, 128.63, 128.19, 128.05, 127.81, 127.73, 127.02, 126.52, 123.28, 122.60, 122.53, 121.60, 119.21, 118.99, 115.06, 114.81, 54.31, 51.87, 36.36. HRMS (ESI): calcd for C₂₈H₂₃BrN₂O₄+Na: 553.0739 found 553.0732.

### (2-(2-Naphthamido)benzoyl)-L-phenylalanine 22a

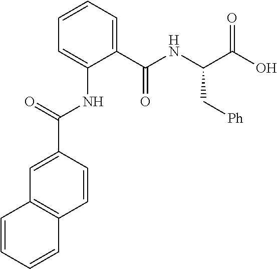 [SMILES: O=C(Nc1ccccc1C(=O)N[C@@H](Cc1ccccc1)C(=O)O)c1ccc2ccccc2c1]

Compound 22a was prepared by General Procedure C using compound 21a (3.5 g, 7.8 mmol) to give 22a as a pure white powder in quantitative yield (3.3 g). IR (cm⁻¹): 3302, 3025, 1684, 1585, 1506, 1442, 1295, 1219, 912, 859, 808, 752, 687. ¹H NMR (400 MHz, DMSO-d₆) 12.32 (1H, s, NH), 8.98-8.77 (1H, bs, NH), 8.64 (1H, dd, J=8.4, 1.2 Hz, ArH), 8.47 (1H, d, J=1.8 Hz, ArH), 8.10 (2H, dd, J=8.2, 3.7 Hz, ArH), 8.02 (1H, dd, J=7.5, 1.8 Hz, ArH), 7.90 (1H, dd, J=8.6, 1.9 Hz, ArH), 7.74 (1H, dd, J=7.9, 1.5 Hz, ArH), 7.70-7.60 (2H, m, ArH), 7.57 (1H, ddd, J=8.6, 7.3, 1.5 Hz, ArH), 7.33-7.25 (2H, m, ArH), 7.25-7.13 (3H, m, ArH), 7.09-6.98 (1H, m, ArH), 4.60 (1H, t, J=6.7 Hz, CH), 3.06 (2H, dd, J=13.7, 9.8 Hz, CH₂). ¹³C NMR (101 MHz, DMSO-d₆) 172.71, 168.79, 164.47, 139.16, 137.97, 134.45, 132.46, 132.23, 131.83, 129.14, 129.07, 128.71, 128.37, 128.16, 127.79, 127.72, 127.10, 126.40, 123.23, 122.90, 120.36, 120.24, 54.14, 36.19. HRMS (ESI): calcd for C₂₇H₂₂N₂O₄+Na: 461.1477 found 461.1471.

### (2-(2-Naphthamido)-4-fluorobenzoyl)-L-phenylalanine 22b

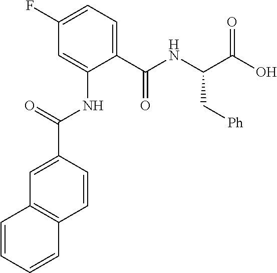 [SMILES: O=C(Nc1cc(F)ccc1C(=O)N[C@@H](Cc1ccccc1)C(=O)O)c1ccc2ccccc2c1]

Compound 22b was prepared by General Procedure C using compound 21b (1.0 g, 2.1 mmol) to afford 22b as a white powder in quantitative yield (0.97 g). IR (cm⁻¹): 3061, 2601, 2341, 1730, 1649, 1605, 1519, 1413, 1309, 1206, 949, 823, 756, 699. ¹H NMR (400 MHz, DMSO-d₆) 11.99 (1H, s, NH), 9.19 (1H, d, J=8.1 Hz, NH), 8.62 (1H, dd, J=9.2, 5.3 Hz, ArH), 8.45 (1H, d, J=1.8 Hz, ArH), 8.10 (2H, d, J=8.5 Hz, ArH), 8.03 (1H, dd, J=7.7, 1.8 Hz, ArH), 7.87 (1H, dd, J=8.6, 1.8 Hz, ArH), 7.72-7.56 (3H, m, ArH), 7.49 (1H, ddd, J=9.2, 8.0, 3.0 Hz, ArH), 7.36-7.27 (2H, m, ArH), 7.21 (2H, t, J=7.6 Hz, ArH), 7.14-6.98 (1H, m, ArH), 4.79-4.64 (1H, m, CH), 3.05 (2H, m, CH₂). ¹³C NMR (101 MHz, DMSO-d₆) 172.48, 167.37, 164.45, 137.91, 135.60, 134.45, 132.20, 131.62, 129.14, 129.05, 128.70, 128.18, 128.14, 127.82, 127.72, 127.11, 126.39, 123.21, 119.03, 114.96, 54.29, 36.28. HRMS (ESI): calcd for C₂₇H₂₁FN₂O₄+Na: 479.1383 found 479.1378.

### (2-(2-Naphthamido)-4-chlorobenzoyl)-L-phenylalanine 22c

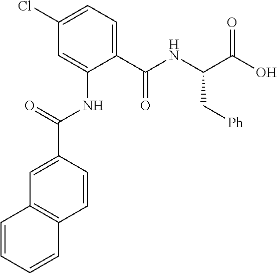 [SMILES: O=C(Nc1cc(Cl)ccc1C(=O)N[C@@H](Cc1ccccc1)C(=O)O)c1ccc2ccccc2c1]

Compound 22c was prepared by General Procedure C using compound 21c (1.1 g, 2.3 mmol) to give 22c in quantitative yield (1.0 g). IR (cm⁻¹): 3313, 2935, 2596, 1726, 1665, 1588, 1521, 1454, 1393, 1308, 1225, 1094, 1014, 973, 913, 812, 759, 703. ¹H NMR (300 MHz, DMSO-d₆) 12.20 (1H, s, NH), 9.04 (1H, s, NH), 8.64 (1H, d, J=9.0 Hz, ArH), 8.45 (1H, d, J=1.8 Hz, ArH), 8.14-8.05 (2H, m, ArH), 8.04-7.97 (1H, m, ArH), 7.87 (1H, dd, J=8.6, 1.8 Hz, ArH), 7.80 (1H, d, J=2.5 Hz, ArH), 7.71-7.57 (3H, m, ArH), 7.34-7.24 (2H, m, ArH), 7.16 (2H, t, J=7.6 Hz, ArH), 7.06-6.94 (1H, m, ArH), 4.60 (1H, s, CH), 3.31-2.93 (4H, m, CH₂). ¹³C NMR (101 MHz, DMSO-d₆) 172.46, 167.35, 164.43, 137.89, 135.58, 134.43, 132.18, 131.60, 129.12, 129.03, 128.68, 128.16, 128.12, 127.80, 127.70, 127.09, 126.37, 123.19, 119.01, 114.94, 54.27, 36.26. HRMS (ESI): calcd for C₂₇H₂₁ClN₂O₄+Na: 495.1088 found 495.1091.

### (2-(2-Naphthamido)-4-bromobenzoyl)-L-phenylalanine 22d

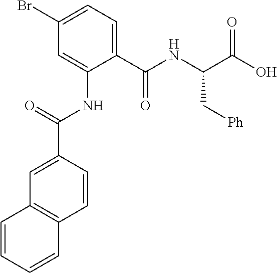 [SMILES: O=C(Nc1cc(Br)ccc1C(=O)N[C@@H](Cc1ccccc1)C(=O)O)c1ccc2ccccc2c1]

Compound 22d was prepared by General Procedure C using compound 21d (1.0 g, 1.9 mmol) to give 22d as a pure brownish solid in quantitative yield (0.97 g). IR (cm⁻¹): 3314, 2935, 2602, 2342, 1728, 1665, 1589, 1521, 1454, 1393, 1307, 1224, 1093, 1014, 973, 913, 813, 758, 703. ¹H NMR (300 MHz, DMSO-d₆) 12.06 (1H, s, NH), 9.31 (1H, d, J=8.2 Hz, NH), 8.58 (1H, d, J=8.9 Hz, ArH), 8.44 (1H, d, J=1.8 Hz, ArH), 8.10 (2H, d, J=8.4 Hz, ArH), 8.06-8.00 (1H, m, ArH), 7.96 (1H, d, J=2.3 Hz, ArH), 7.86 (1H, dd, J=8.6, 1.8 Hz, ArH), 7.79 (1H, dd, J=9.0, 2.3 Hz, ArH), 7.66 (2H, ddd, J=6.9, 4.5, 1.8 Hz, ArH), 7.34-7.26 (2H, m, ArH), 7.20 (2H, t, J=7.6 Hz, ArH), 7.11-7.03 (1H, m, ArH), 4.75 (1H, ddd, J=10.6, 7.9, 4.5 Hz, CH), 3.18-2.95 (2H, m, CH₂). ¹³C NMR (101 MHz, DMSO-d₆) 172.43, 167.32, 164.40, 137.86, 135.55, 134.40, 132.15, 131.57, 129.09, 129.00, 128.65, 128.13, 128.09, 127.77, 127.67, 127.06, 126.34, 123.16, 118.98, 114.91, 54.24, 36.23. HRMS (ESI): calcd for C₂₇H₂₁BrN₂O₄+Na: 539.0582 found 539.0586.

### Tert-butyl(S)-(2-(2-(2-(2-naphthamido)benzamido)-3-phenylpropanamido)ethyl)-carbamate 23

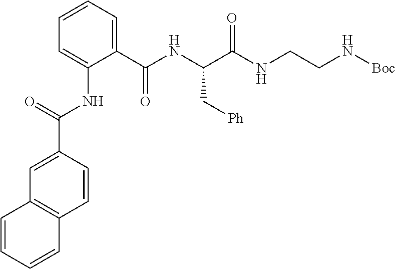 [SMILES: CC(C)(C)OC(=O)NCCNC(=O)[C@H](Cc1ccccc1)NC(=O)c1ccccc1NC(=O)c1ccc2ccccc2c1]

Compound 23 was prepared by General Procedure D using compound 22a (0.7 g, 1.6 mmol) and tert-butyl (2-aminoethyl)carbamate (1.1 equivalents) to give 23 as an off-white solid (0.5 g, 54% yield). IR (cm⁻¹): 3311, 2927, 1683, 1590, 1517, 1445, 1366, 1274, 1249, 1167, 989, 911, 858, 754, 681. ¹H NMR (400 MHz, DMSO-d₆) 12.11 (1H, s, NH), 9.00 (1H, d, J=8.4 Hz, NH), 8.56 (1H, dd, J=8.3, 1.2 Hz, ArH), 8.44 (1H, m, NH), 8.23 (1H, t, J=5.7 Hz, ArH), 8.12-8.05 (2H, m, ArH), 8.03 (1H, dd, J=7.7, 1.7 Hz, ArH), 7.88 (1H, dd, J=8.6, 1.8 Hz, ArH), 7.78 (1H, dd, J=8.0, 1.6 Hz, ArH), 7.62-7.69 (2H, m, ArH), 7.57 (1H, ddd, J=8.6, 7.4, 1.5 Hz, ArH), 7.37-7.27 (2H, m, ArH), 7.27-7.15 (3H, m, ArH), 7.10-6.99 (1H, m, ArH), 6.77 (1H, t, J=5.5 Hz, NH), 4.80-4.65 (1H, m, CH), 3.23-2.90 (6H, m, CH₂), 1.33 (9H, s, CH₃). ¹³C NMR (101 MHz, DMSO-d₆) 170.86, 168.61, 164.51, 155.63, 138.96, 138.33, 134.42, 132.20, 131.84, 129.11, 129.07, 128.59, 128.55, 128.12, 128.01, 127.84, 127.72, 127.07, 126.23, 123.29, 122.88, 121.00, 120.45, 77.67, 54.90, 37.21, 28.18. HRMS (ESI): calcd for C₃₄H₃₆N₄O₅+Na: 603.2583 found 603.2580.

### Tert-Butyl (S)-(3-(2-(2-(2-naphthamido)benzamido)-3-phenylpropanamido)propyl)-carbamate 24

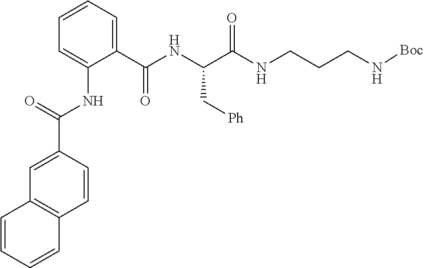 [SMILES: CC(C)(C)OC(=O)NCCCNC(=O)[C@H](Cc1ccccc1)NC(=O)c1ccccc1NC(=O)c1ccc2ccccc2c1]

Compound 24 was prepared by General Procedure D using compound 22a (1.0 g, 2.3 mmol) and tert-butyl (2-aminoethyl)carbamate (1.1 equivalents) to give 24 as a white solid (0.68 g, 50% yield). IR (cm⁻¹): 3290, 3059, 2927, 2115, 1945, 1650, 1588, 1511, 1442, 1388, 1283, 1246, 1166, 1043, 912, 860, 749, 682. ¹H NMR (300 MHz, DMSO-d₆) 12.12 (1H, s, NH), 9.02 (1H, d, J=8.4 Hz, NH), 8.56 (1H, dd, J=8.4, 1.1 Hz, NH), 8.45 (1H, d, J=1.8 Hz, ArH), 8.15 (1H, t, J=5.8 Hz, NH), 8.08 (2H, d, J=8.4 Hz, ArH), 8.03 (1H, dd, J=7.9, 1.8 Hz, ArH), 7.87 (1H, dd, J=8.7, 1.8 Hz, ArH), 7.78 (1H, dd, J=8.0, 1.5 Hz, ArH), 7.72-7.63 (2H, m, ArH), 7.62-7.50 (1H, m, ArH), 7.41-7.29 (2H, m, ArH), 7.27-7.14 (3H, m, ArH), 7.09-7.00 (1H, m, ArH), 6.74 (1H, t, J=5.7 Hz, NH), 4.79-4.63 (1H, m, CH), 3.25-2.82 (6H, m, CH₂), 1.63-1.41 (2H, m, CH₂), 1.32 (9H, s, CH₃). ¹³C NMR (101 MHz, DMSO-d₆). 170.86, 168.61, 164.51, 155.63, 138.96, 138.33, 134.42, 132.20, 131.84, 129.11, 129.07, 128.59, 128.55, 128.12, 128.01, 127.84, 127.72, 127.07, 126.23, 123.29, 122.88, 121.00, 120.45, 77.67, 54.90, 37.21, 28.18. HRMS (ESI): calcd for C₃₅H₃₈N₄O₅+Na: 617.2740 found 617.2742.

### (S)—N-(2-((1-((2-aminoethyl)amino)-1-oxo-3-phenylpropan-2-yl)carbamoyl)phenyl)-2-naphthamide 10a

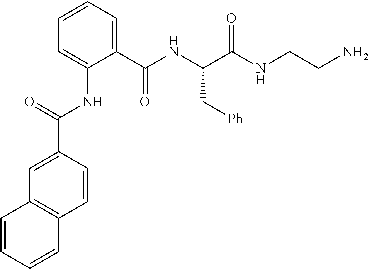 [SMILES: NCCNC(=O)[C@H](Cc1ccccc1)NC(=O)c1ccccc1NC(=O)c1ccc2ccccc2c1]

Compound 10a was prepared by General Procedure E using compound 23 (0.4 g, 6.9 mmol) to afford 10a in quantitative yield (0.33 g). IR (cm⁻¹): 3295, 2928, 1657, 1587, 1516, 1444, 1301, 1177, 1128, 955, 911, 835, 754, 698. ¹H NMR (400 MHz, DMSO-d₆) 12.05 (1H, s, NH), 9.02 (1H, d, J=8.4 Hz, NH), 8.54 (1H, dd, J=8.4, 1.2 Hz, ArH), 8.45 (1H, m, NH), 8.36 (1H, t, J=5.7 Hz, ArH), 8.13-7.97 (3H, m, ArH), 7.88-7.83 (1H, m, ArH), 7.81-7.77 (1H, m, ArH), 7.70-7.64 (3H, m, ArH), 7.64-7.55 (1H, m, ArH), 7.36-7.26 (2H, m, ArH), 7.27-7.14 (3H, m, ArH), 7.10-6.98 (1H, m, ArH), 4.79 (ddd, J=10.7, 8.4, 4.3 Hz, CH), 3.26-2.92 (4H, m, CH₂), 2.84 (2H, t, J=6.7 Hz, CH₂), 1.52 (2H, bs, NH₂). ¹³C NMR (101 MHz, DMSO) 171.48, 168.61, 164.54, 138.87, 138.15, 134.43, 132.28, 132.21, 131.83, 129.10, 128.61, 128.53, 128.18, 128.06, 127.88, 127.75, 127.14, 126.30, 123.27, 122.97, 121.09, 120.60, 54.68, 38.45, 37.04, 36.59. HRMS (ESI): calcd for C₂₉H₂₈N₄O₃+Na: 503.2059 found 503.2054.

### (S)—N-(2-((1-((3-aminopropyl)amino)-1-oxo-3-phenylpropan-2-yl)carbamoyl)phenyl)-2-naphthamide 11a

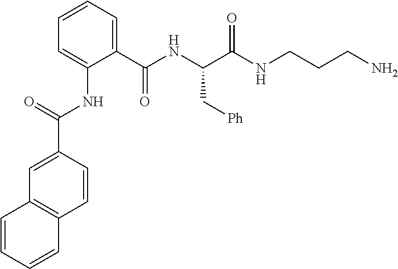 [SMILES: NCCCNC(=O)[C@H](Cc1ccccc1)NC(=O)c1ccccc1NC(=O)c1ccc2ccccc2c1]

Compound 11a was prepared by General Procedure E using compound 24 (0.65 g, 1.1 mmol) to afford 11a as a pure white solid in quantitative yield (0.54 g). IR (cm⁻¹): 3285, 3050, 2944, 1652, 1590, 1506, 1441, 1389, 1291, 1181, 1129, 1046, 1033, 913, 836. ¹H NMR (400 MHz, DMSO-d₆) 12.11 (1H, s, NH), 9.06 (1H, d, J=8.3 Hz, NH), 8.55 (1H, d, J=8.4 Hz, ArH), 8.46 (1H, d, J=1.8 Hz, NH), 8.34 (1H, t, J=5.8 Hz, ArH), 8.09 (2H, d, J=8.3 Hz, ArH), 8.04 (1H, dd, J=7.4, 1.9 Hz, ArH), 7.88 (1H, dd, J=8.6, 1.8 Hz, ArH), 7.79 (1H, dd, J=8.0, 1.5 Hz, ArH), 7.67 (1H, m, ArH), 7.58 (1H, td, J=8.2, 7.8, 1.5 Hz, ArH), 7.38-7.30 (2H, m, ArH), 7.26-7.15 (3H, m, ArH), 7.11-7.02 (1H, m, ArH), 4.75 (1H, ddd, J=10.7, 8.3, 4.4 Hz, CH), 3.22-2.92 (4H, m, CH₂), 2.85-2.61 (2H, m, CH₂), 1.66 (2H, m, CH₂), 1.52 (2H, bs, NH₂). ¹³C NMR (101 MHz, DMSO) 171.21, 168.65, 164.54, 138.92, 138.13, 134.42, 132.28, 132.20, 131.84, 129.09, 129.07, 128.60, 128.16, 128.07, 127.87, 127.74, 127.12, 126.32, 123.25, 122.95, 121.02, 120.58, 54.91, 37.17, 36.71, 35.74, 27.35. HRMS (ESI): calcd for C₃₀H₃₀N₄O₃+Na: 517.2216 found 517.2214.

### (S)—N-(2-((1-((3-(dimethylamino)propyl)amino)-1-oxo-3-phenylpropan-2-yl)carbamoyl)-phenyl)-2-naphthamide 12a

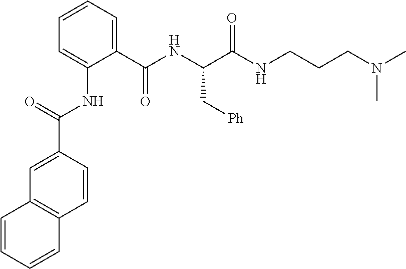 [SMILES: CN(C)CCCNC(=O)[C@H](Cc1ccccc1)NC(=O)c1ccccc1NC(=O)c1ccc2ccccc2c1]

Compound 12a was prepared by General Procedure D using compound 22a (1.5 g, 3.4 mmol) with N¹,N¹-dimethylpropane-1,3-diamine (1.1 equivalents) to afford 12a as a pure white solid (1.3 g, 78% yield). IR (cm-1): 3289, 3234, 3056, 2935, 2622, 2485, 2343, 2114, 1935, 1656, 1587, 1503, 1432, 1287, 1234, 1028, 910, 859, 751. ¹H NMR (400 MHz, DMSO-d₆) 12.13 (1H, s, NH), 9.05 (1H, d, J=8.4 Hz, NH), 8.54 (1H, dd, J=8.4, 1.2 Hz, ArH), 8.46 (1H, d, J=1.8 Hz, ArH), 8.26 (1H, t, J=5.7 Hz, NH), 8.09 (2H, d, J=8.2 Hz, ArH), 8.03 (1H, dd, J=7.5, 1.9 Hz, ArH), 7.88 (1H, dd, J=8.6, 1.8 Hz, ArH), 7.79 (1H, dd, J=7.9, 1.5 Hz, ArH), 7.66 (2H, tt, J=7.0, 5.3 Hz, ArH), 7.58 (1H, td, J=8.4, 7.9, 1.5 Hz, ArH), 7.42-7.29 (2H, m, ArH), 7.28-7.15 (3H, m, ArH), 7.13-6.97 (1H, m, ArH), 4.73 (1H, ddd, J=10.7, 8.3, 4.4 Hz, CH), 3.18-2.96 (4H, m, CH₂), 2.43 (2H, t, J=7.5 Hz, CH₂), 2.23 (6H, s, CH₃), 1.59 (2H, td, J=7.0, 3.0 Hz, CH₂). ¹³C NMR (101 MHz, DMSO) 170.59, 168.62, 164.55, 138.92, 138.32, 134.43, 132.21, 131.84, 129.13, 129.06, 128.60, 128.53, 128.13, 128.04, 127.82, 127.73, 127.08, 126.26, 123.29, 122.93, 121.11, 120.56, 56.22, 55.02, 37.12, 36.89, 26.36. HRMS (ESI): calcd for C₃₂H₃₄N₄O₃+Na: 545.2529 found 545.2523.

### (S)—N-(2-((1-((3-(dimethylamino)propyl)amino)-1-oxo-3-phenylpropan-2-yl)carbamoyl)-5-fluorophenyl)-2-naphthamide 15a

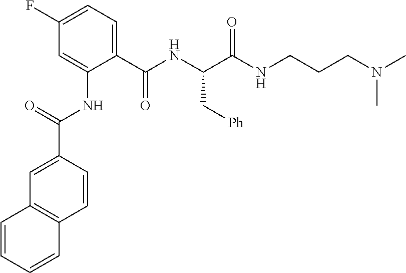 [SMILES: CN(C)CCCNC(=O)[C@H](Cc1ccccc1)NC(=O)c1ccc(F)cc1NC(=O)c1ccc2ccccc2c1]

Compound 15a was prepared by General Procedure D using compound 22b (0.9 g, 2.0 mmol) with N¹, N¹-dimethylpropane-1,3-diamine (1.1 equivalents) to give 15a as a pure white solid (0.78 g, 72% yield). IR (cm⁻¹): 3292, 3230, 3055, 2950, 2609, 2478, 2343, 2114, 1657, 1601, 1521, 1411, 1300, 1241, 1203, 1099, 945, 871, 824, 754, 677. ¹H NMR (400 MHz, DMSO-d₆) 11.89 (1H, s, NH), 9.16 (1H, d, J=8.4 Hz, NH), 8.50 (1H, dd, J=9.2, 5.3 Hz, ArH) 8.45 (1H, d, J=1.8 Hz, ArH), 8.31 (1H, t, J=5.8 Hz, NH), 8.16-8.05 (2H, m, ArH), 8.03 (1H, dd, J=7.7, 1.9 Hz, ArH), 7.87 (1H, dd, J=8.6, 1.8 Hz, ArH), 7.76-7.56 (3H, m, ArH), 7.47 (1H, ddd, J=9.2, 8.0, 3.0 Hz, ArH), 7.38-7.29 (2H, m, ArH), 7.20 (2H, t, J=7.6 Hz, ArH), 7.13-6.99 (1H, m, ArH), 4.73 (1H, ddd, J=10.8, 8.3, 4.5 Hz, CH), 3.24-3.15 (2H, m, CH₂), 3.14-2.91 (2H, m, CH₂), 2.61 (2H, d, J=13.7, 10.7 Hz, CH₂), 2.39 (6H, s, CH₃), 1.65 (2H, td, J=7.1, 2.7 Hz, CH₂). ¹³C NMR (101 MHz, DMSO-d₆) 170.60, 167.36, 167.34, 164.57, 158.32, 155.93, 138.16, 135.25, 135.23, 134.44, 132.18, 131.64, 129.10, 129.06, 128.60, 128.16, 128.05, 127.87, 127.73, 127.11, 126.31, 123.28, 123.15, 123.08, 122.93, 118.95, 118.74, 115.30, 115.06, 55.23, 55.01, 48.59, 43.17, 37.11, 36.33, 25.16. HRMS (ESI): calcd for C₃₂H₃₃FN₄O₃+Na: 563.2434 found 563.2431.

### (S)—N-(5-chloro-2-((1-((3-(dimethylamino)propyl)amino)-1-oxo-3-phenylpropan-2-yl)carbamoyl)phenyl)-2-naphthamide 16a

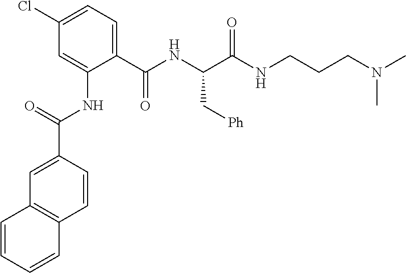 [SMILES: CN(C)CCCNC(=O)[C@H](Cc1ccccc1)NC(=O)c1ccc(Cl)cc1NC(=O)c1ccc2ccccc2c1]

Compound 16a was prepared by General Procedure D using compound 22c (0.90 g, 1.9 mmol) with N¹,N¹-dimethylpropane-1,3-diamine (1.1 equivalents) to give 16a as a pure off-white solid (0.78 g, 74% yield). IR (cm⁻¹): 3290, 3053, 2928, 2170, 1654, 1583, 1505, 1442, 1399, 1301, 1089, 913, 811, 755, 702. ¹H NMR (300 MHz, DMSO-d₆) 11.97 (1H, s, NH), 9.22 (1H, d, J=8.3 Hz, NH), 8.53 (1H, d, J=8.9 Hz, ArH), 8.44 (1H, d, J=1.8 Hz, ArH), 8.31 (1H, t, J=5.8 Hz, NH), 8.09 (2H, d, J=8.5 Hz, ArH), 8.06-7.97 (1H, m, ArH), 7.91-7.77 (2H, m, ArH), 7.74-7.57 (3H, m, ArH), 7.32 (2H, d, J=7.2 Hz, ArH), 7.19 (2H, t, J=7.5 Hz, ArH), 7.05 (1H, t, J=7.3 Hz, ArH), 4.73 (1H, ddd, J=10.7, 8.2, 4.5 Hz, CH), 3.22-2.89 (4H, m, CH₂), 2.63 (2H, t, J=7.5 Hz, CH₂), 2.40 (6H, s, CH₃), 1.75-1.56 (2H, m, CH₂). ¹³C NMR (76 MHz, DMSO-d₆) 170.65, 167.37, 164.66, 138.15, 137.70, 134.51, 132.19, 131.90, 131.54, 129.12, 128.68, 128.29, 128.07, 127.96, 127.77, 127.19, 126.80, 126.35, 123.26, 122.82, 122.40, 55.28, 55.00, 43.22, 37.12, 36.35. HRMS (ESI): calcd for C₃₂H₃₃ClN₄O₃+Na: 579.2139 found 579.2132.

### (S)—N-(5-bromo-2-((1-((3-(dimethylamino)propyl)amino)-1-oxo-3-phenylpropan-2-yl)carbamoyl)phenyl)-2-naphthamide 17a

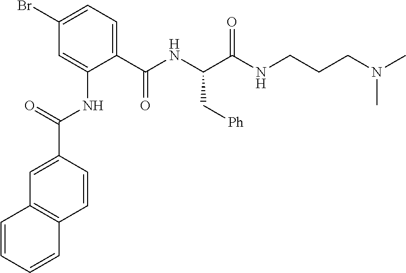 [SMILES: CN(C)CCCNC(=O)[C@H](Cc1ccccc1)NC(=O)c1ccc(Br)cc1NC(=O)c1ccc2ccccc2c1]

Compound 17a was prepared by General Procedure D using compound 22d (0.9 g, 1.7 mmol) with N¹,N¹-dimethylpropane-1,3-diamine (1.1 equivalents) to give 17a as a brownish white solid (0.73 g, 70% yield). IR (cm⁻¹): 3296, 3055, 2927, 1654, 1583, 1505, 1441, 1395, 1303, 1090, 912, 812, 755, 704. ¹H NMR (300 MHz, DMSO-d₆) 11.97 (1H, s, NH), 9.22 (1H, d, J=8.3 Hz, NH), 8.50-8.41 (2H, m, ArH), 8.31 (1H, t, J=5.8 Hz, NH), 8.12-8.06 (2H, m, ArH), 8.06-8.01 (1H, m, ArH), 7.96 (1H, d, J=2.3 Hz, ArH), 7.85 (1H, dd, J=8.6, 1.8 Hz, ArH), 7.77 (1H, dd, J=8.9, 2.3 Hz, ArH), 7.73-7.57 (2H, m, ArH), 7.38-7.26 (2H, m, ArH), 7.19 (2H, dd, J=8.3, 6.9 Hz, ArH), 7.11-6.98 (1H, m, ArH), 4.73 (1H, td, J=9.4, 8.3, 4.5 Hz, CH), 3.24-2.88 (4H, m, CH₂), 2.63-2.69 (2H, m, CH₂), 2.44 (6H, s, CH₃), 1.74-1.53 (2H, m, CH₂). ¹³C NMR (76 MHz, DMSO-d₆) 170.70, 167.29, 164.66, 138.19, 138.11, 134.78, 134.52, 132.19, 131.55, 131.16, 129.17, 129.13, 128.70, 128.28, 128.06, 127.98, 127.79, 127.20, 126.34, 123.27, 123.10, 122.64, 114.78, 55.02, 42.86, 37.16, 36.28, 24.87. HRMS (ESI): calcd for C₃₂H₃₃BrN₄O₃+Na: 623.1634 found 623.1629.

### (S)-3-(2-(2-(2-naphthamido)benzamido)-3-phenylpropanamido)-N,N,N-trimethylpropan-1-aminium iodide 13

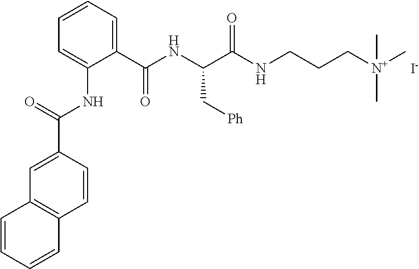 [SMILES: C[N+](C)(C)CCCNC(=O)[C@H](Cc1ccccc1)NC(=O)c1ccccc1NC(=O)c1ccc2ccccc2c1 ; [I-]]

Compound 12a (0.3 g, 0.6 mmol) was suspended in anhydrous THF under argon atmosphere. After cooling to 0° C., methyl iodide (2.0 equivalents) was added dropwise and the reaction mixture was stirred at room temperature for 16 h. After completion, the reaction mixture was concentrated under reduced pressure to remove solvent. The resulting crude material was washed with cold DCM to afford 13 as a pure yellowish white solid (0.29 g, 95% yield). IR (cm⁻¹): 3251, 3033, 2940, 2620, 1657, 1597, 1510, 1442, 1304, 1119, 960, 911, 824, 751. ¹H NMR (300 MHz, DMSO-d₆) 12.10 (1H, s, NH), 9.62 (1H, bs, NH), 9.06 (1H, d, J=8.3 Hz, NH), 8.54 (1H, d, J=8.3 Hz, ArH), 8.46 (1H, d, J=1.8 Hz, ArH), 8.09 (2H, d, J=8.4 Hz, ArH, 8.06-7.99 (1H, m, ArH), 7.88 (1H, dd, J=8.6, 1.8 Hz, ArH), 7.80 (1H, dd, J=8.0, 1.5 Hz, ArH), 7.72-7.63 (2H, m, ArH), 7.63-7.51 (1H, m, ArH), 7.34 (2H, d, J=7.2 Hz, ArH), 7.22 (3H, td, J=7.6, 3.0 Hz, ArH), 7.07 (1H, t, J=7.3 Hz, ArH), 4.84-4.66 (1H, m, CH), 3.26-2.85 (6H, m, CH₂), 2.67 (9H, s, CH₃), 1.73 (2H, m, CH₂). ¹³C NMR (76 MHz, DMSO-d₆) 170.59, 168.62, 164.55, 138.92, 138.32, 134.43, 132.21, 131.84, 129.13, 129.06, 128.60, 128.53, 128.13, 128.04, 127.82, 127.73, 127.08, 126.26, 123.29, 122.93, 121.11, 120.56, 56.22, 55.02, 37.12, 36.89, 26.36. HRMS (ESI): calcd for C₃₃H₃₇N₄O₃: 537.2860 found 537.2858.

### (S)—N-(2-((1-((3-Di-Bocguanidinopropyl)amino)-1-oxo-3-phenylpropan-2-yl)carbamoyl)-phenyl)-2-naphthamide 25

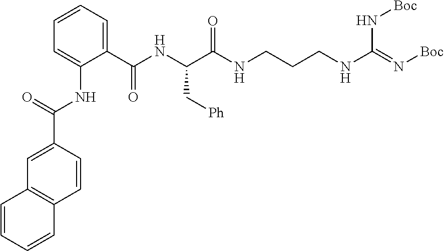 [SMILES: CC(C)(C)OC(=O)/N=C(/NCCCNC(=O)[C@H](Cc1ccccc1)NC(=O)c1ccccc1NC(=O)c1ccc2ccccc2c1)NC(=O)OC(C)(C)C]

The primary amine 11a (0.28 g, 0.57 mmol) was dissolved in anhydrous DCM under nitrogen atmosphere. N, N′-Di-Boc-1H-pyrazole-1-carboxamidine (1.1. equivalent) and trimethylamine (2.0 equivalent) were added to the reaction mixture. After completion of the reaction (18 h), the reaction mixture was removed under reduced pressure and the resulting crude material was subjected to column chromatography with DCM:MeOH (5%) as the mobile phase. Compound 25 was obtained as a pure white solid in (0.4 g, 98% yield). IR (cm⁻¹): 3217, 3036, 2923, 2627, 2320, 2097, 1909, 165, 1579, 1515, 1446, 1274, 1205, 960, 934, 839, 753, 698. ¹H NMR (400 MHz, DMSO-d₆) 12.15 (1H, s, NH), 11.45 (1H, s, NH), 9.04 (1H, d, J=8.2 Hz, ArH), 8.57 (1H, dd, J=8.4, 1.2 Hz, ArH), 8.48-8.41 (1H, bs, NH), 8.31 (1H, t, J=5.6 Hz, NH), 8.25 (1H, t, J=5.9 Hz, ArH), 8.06 (2H, dd, J=9.1, 2.2 Hz, ArH), 8.01 (1H, dd, J=7.6, 1.8 Hz, ArH), 7.87 (1H, dd, J=8.6, 1.8 Hz, ArH), 7.78 (1H, dd, J=8.0, 1.5 Hz, ArH), 7.70-7.62 (2H, m, ArH), 7.62-7.52 (1H, m, ArH), 7.39-7.31 (2H, m, ArH), 7.21 (2H, t, J=7.5 Hz, ArH), 7.11-7.03 (1H, m, ArH), 4.72 (1H, ddd, J=10.4, 8.1, 4.6 Hz, CH), 3.27-3.01 (6H, m, CH₂), 3.01-2.94 (1H, m, NH) 1.67-1.50 (2H, m, CH₂), 1.44 (9H, s CH₃), 1.35 (9H, s, CH₃). ¹³C NMR (101 MHz, DMSO-d₆) 170.81, 170.73, 168.74, 168.67, 164.58, 163.13, 155.32, 151.95, 139.01, 138.95, 138.27, 134.46, 132.23, 131.87, 129.18, 129.08, 128.64, 128.58, 128.09, 128.07, 127.84, 127.78, 127.74, 127.13, 127.07, 126.32, 123.28, 122.94, 121.12, 121.03, 120.53, 82.83, 78.16, 67.00, 55.18, 55.03, 54.93, 31.33, 28.28, 28.00, 27.63. HRMS (ESI): calcd for C₄₁H₄₈N₆O₇+H: 737.3663 found 737.3668.

### (S)—N-(2-((1-((3-guanidinopropyl)amino)-1-oxo-3-phenylpropan-2-yl)carbamoyl)-phenyl)-2-naphthamide 14a

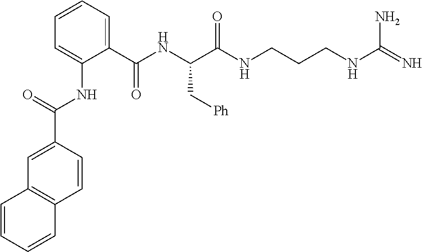 [SMILES: N=C(N)NCCCNC(=O)[C@H](Cc1ccccc1)NC(=O)c1ccccc1NC(=O)c1ccc2ccccc2c1]

Compound 14a was prepared by General Procedure E using compound 25 (0.35 g, 0.41 mmol) to afford 14a as an off-white solid in quantitative yield (0.21 g). IR (cm⁻¹): 3270, 3035, 2937, 2343, 2113, 1735, 1687, 1576, 1512, 1432, 1273, 1204, 1162, 1062, 917, 840, 753. ¹H NMR (400 MHz, DMSO-d₆) 11.25 (1H, s, NH), 8.84 (1H, d, J=8.2 Hz, NH), 8.37 (1H, dd, J=8.4, 1.2 Hz, ArH), 8.28-8.21 (1H, m, ArH), 8.11 (1H, t, J=5.6 Hz, NH), 8.05 (1H, t, J=5.9 Hz, ArH), 7.86 (2H, dd, J=9.1, 2.2 Hz, ArH), 7.84 (1H, bs, NH), 7.81 (1H, dd, J=7.6, 1.8 Hz, ArH), 7.67 (1H, dd, J=8.6, 1.8 Hz, ArH), 7.58 (1H, dd, J=8.0, 1.5 Hz, ArH), 7.50-7.42 (2H, m, ArH), 7.42-7.32 (1H, m, ArH), 7.19-7.11 (m, 2H), 7.01 (t, J=7.5 Hz, 2H), 6.99 (2H, m, NH), 6.91-6.83 (1H, m, ArH), 4.52 (1H, ddd, J=10.4, 8.1, 4.6 Hz, CH), 3.07-2.74 (6H, m, CH₂), 2.64 (1H, bs, NH), 1.47-1.30 (2H, m, CH₂). ¹³C NMR (101 MHz, DMSO-d₆) 170.81, 170.73, 168.74, 168.67, 155.32, 151.95, 139.01, 138.95, 138.27, 134.46, 132.23, 131.87, 129.18, 129.08, 128.64, 128.58, 128.09, 128.07, 127.84, 127.78, 127.74, 127.13, 127.07, 126.32, 123.28, 122.94, 121.12, 121.03, 120.53, 82.83, 78.16, 67.00, 55.18, 55.03, 54.93, 31.33, 28.00, 27.63. HRMS (ESI): calcd for C₃₁H₃₂N₆O₃+Na: 559.2434 found 559.2330.

### (S)-3-(2-(2-(2-naphthamido)benzamido)-3-phenylpropanamido)-N,N-dimethylpropan-1-aminium Ttrifluoroacetate 18

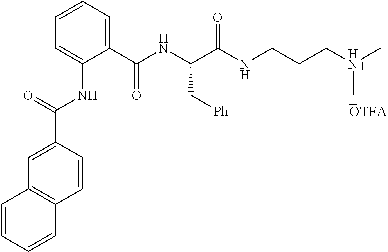

Compound 12a (0.25 g, 0.48 mmol) was dissolved in DCM and cooled to 0° C. TFA (1.5 equivalent) was added and the reaction mixture was stirred for 30 minutes. Subsequently this mixture was concentrated under reduced pressure. The resulting pale brown crude material was washed with diethyl ether (2×15 mL), filtered, and dried to afford 18 as an off-white solid in quantitative yield (0.35 g). IR (cm⁻¹): 3287, 3056, 2955, 2721, 2649, 2343, 2121, 1670, 1587, 1507, 1442, 1300, 1173, 1123, 980, 912, 798, 750, 698. ¹H NMR (300 MHz, DMSO-d₆) 12.10 (1H, s, NH), 9.62 (1H, bs, NH), 9.06 (1H, d, J=8.3 Hz, NH), 8.54 (1H, d, J=8.3 Hz, ArH), 8.46 (1H, d, J=1.8 Hz, ArH), 8.36 (1H, t, J=5.8 Hz, NH), 8.09 (2H, d, J=8.4 Hz, ArH, 8.06-7.99 (1H, m, ArH), 7.88 (1H, dd, J=8.6, 1.8 Hz, ArH), 7.80 (1H, dd, J=8.0, 1.5 Hz, ArH), 7.72-7.63 (2H, m, ArH), 7.63-7.51 (1H, m, ArH), 7.34 (2H, d, J=7.2 Hz, ArH), 7.22 (3H, td, J=7.6, 3.0 Hz, ArH), 7.07 (1H, t, J=7.3 Hz, ArH), 4.84-4.66 (1H, m, CH), 3.26-2.85 (6H, m, CH₂), 2.66 (6H, s, CH₃), 1.73 (2H, m, CH₂). ¹³C NMR (101 MHz, DMSO-d₆) 170.57, 168.60, 164.53, 138.90, 138.30, 134.41, 132.19, 131.82, 129.11, 129.04, 128.58, 128.51, 128.11, 128.02, 127.80, 127.71, 127.06, 126.24, 123.27, 122.91, 121.09, 120.54, 56.20, 55.00, 37.10, 36.87, 26.34. HRMS (ESI): calcd for C₃₂H₃₄N₄O₃+H 523.2704 found 523.2711.

### (S)-3-(2-(2-(2-naphthamido)benzamido)-3-phenylpropanamido)-N,N-dimethylpropan-1-aminium Chloride 19

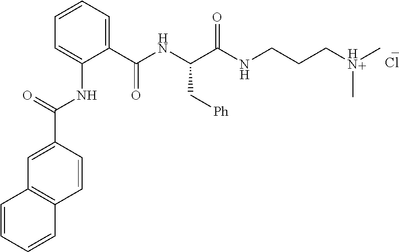

Compound 12a (0.3 g, 0.57 mmol) was dissolved in DCM and the solution cooled down to 0° C. and 4M HCl in dioxane (1.5 equivalent) was added dropwise. The reaction mixture was stirred at room temperature for 1 hour. The resulting precipitate was filtered, dried, and washed with diethyl ether to provided 19 as a white solid in quantitative yield (0.31 g). IR (cm⁻¹): 3284, 3232, 3053, 2933, 2621, 2485, 2343, 2114, 1935, 1656, 1587, 1503, 1432, 1287, 1234, 1028, 910, 859, 670. ¹H NMR (400 MHz, DMSO-d₆) 12.00 (1H s, NH), 9.52 (1H, s, NH), 8.96 (1H, d, J=8.3 Hz, NH), 8.44 (1H, d, J=8.3 Hz, ArH), 8.36 (1H, d, J=1.8 Hz, ArH), 8.26 (1H, t, J=5.8 Hz, NH), 7.99 (2H, d, J=8.4 Hz, ArH), 7.96-7.89 (1H, m, ArH), 7.78 (1H, dd, J=8.6, 1.8 Hz, ArH), 7.70 (1H, dd, J=8.0, 1.5 Hz, ArH), 7.62-7.51 (2H, m, ArH), 7.52-7.42 (1H, m, ArH), 7.24 (2H, d, J=7.2 Hz, ArH), 7.12 (3H, td, J=7.5, 3.0 Hz, ArH), 6.97 (1H, t, J=7.3 Hz, ArH), 4.65 (1H, ddd, J=10.5, 8.1, 4.5 Hz, CH), 3.18-2.71 (6H, m, CH₂), 2.56 (6H, s, CH₃), 1.70-1.49 (2H, m, CH₂). ¹³C NMR (101 MHz, DMSO-d₆) 170.55, 168.58, 164.51, 138.88, 138.28, 134.39, 132.17, 131.80, 129.09, 129.02, 128.56, 128.49, 128.09, 128.00, 127.78, 127.69, 127.04, 126.22, 123.25, 122.89, 121.07, 120.52, 56.18, 54.98, 37.08, 36.85, 26.32. HRMS (ESI): calcd for C₃₂H₃₄N₄O₃+H 523.2704 found 523.2711.

### Example 2—Hydrogel Formation

The formation of self-assembled hydrogels is mainly driven by non-covalent interactions among the hydrogelators in water. To alter the thermodynamic equilibrium, which is required to initiate the self-assembly process, several physical or chemical triggers can be employed. Triggers include cooling from an elevated temperature (temperature switch), changing the pH (pH switch), changing solvents (solvent switch), or the addition of salts.

The simplest method to determine whether a suitable hydrogel has formed is to use the vial inversion test. Through visual inspection, a material can be categorised as a solution, viscous solution, partial gel, or solid-like gel. A stable hydrogel needs to be able to hold its weight and/or maintain its shape during this assessment.

The critical gel concentration (CGC) is defined as the minimum mass of a short peptide required to provide a self-supporting hydrogel per unit volume in the vial inversion test (measured as % w/v).

### NO-Releasing Short Peptides

Pre-weighed samples of short peptide 4 were prepared in aluminium-covered glass vials and Milli-Q water and 2 equivalents of NaOH (1.0 M) were added to make up concentrations of 1% w/v. The resulting suspensions were vortexed for 5 minutes to provide clear yellow solutions with pH 11.

To create a high ionic strength environment, 1 equivalent of NaCl was added and the vial was left to stand without disturbance for 3 hours at room temperature. Meanwhile, as an attempt to form a hydrogel by lowering the pH, 2 equivalents of GdL were added to a high pH solution of 4 followed by gentle mixing. These vials were inverted and allowed to stand overnight to confirm the hydrogel formation.

### Solvent Switch

The formation of hydrogel 4 via the solvent switch method was conducted by dissolving short peptide 4, in an aluminium covered glass vial, with either 50% methanol, ethanol, or dimethyl sulfoxide (DMSO). Respective amounts of Mili-Q water were added to make up concentrations of 1% w/v.

In addition, gelation time was defined as the time (hours) required for 1% w/v of short peptide 4 to form a hydrogel which can be turned upside down without collapsing.

Initially, hydrogel formation was investigated in the presence of an organic co-solvent. Short peptide 4 was dissolved in either methanol, ethanol, or DMSO, followed by addition of the same volume of Milli-Q water and gentle mixing. These mixtures were then subjected to the vial inversion test by simply turning the vial upside down. Using this method, short peptide 4 successfully formed a self-supporting hydrogel (Table 1). However, due to the potential toxicity of organic solvents for biomedical applications, an attempt was made to reduce the proportion of organic co-solvent to 5%. However, this resulted in precipitation of short peptide 4. Therefore, another method to form a hydrogel of short peptide 4 was required.

TABLE 1
Hydrogel formation of nitrobenzene-appended short
peptide 4 via the solvent switch method.

| Entry | Co-solvent | Ratio^{a} | CGC^{b} | Time | Remarks^{d} |
|---|---|---|---|---|---|
| 1 | MeOH | 50 | 0.4 | Immed. | OG |
| 2 | | 5 | n.a. | Immed. | P |
| 3 | EtOH | 50 | 0.4 | Immed. | OG |
| 4 | | 5 | n.a. | Immed. | P |
| 5 | DMSO | 50 | 0.4 | Immed. | OG |
| 6 | | 5 | n.a. | Immed. | P |

^{a} [symbol] Compared [symbol] with [symbol] volume [symbol] of [symbol] Milli-Q [symbol] water, [symbol] denoted [symbol] as [symbol] % v/v
^{b} [symbol] CGC = [symbol] Critical [symbol] gel [symbol] concentration, [symbol] denoted [symbol] as [symbol] % w/v. [symbol]
^{d} [symbol] OG = [symbol] opaque [symbol] gel; [symbol] P = [symbol] precipitate

### Addition of Base

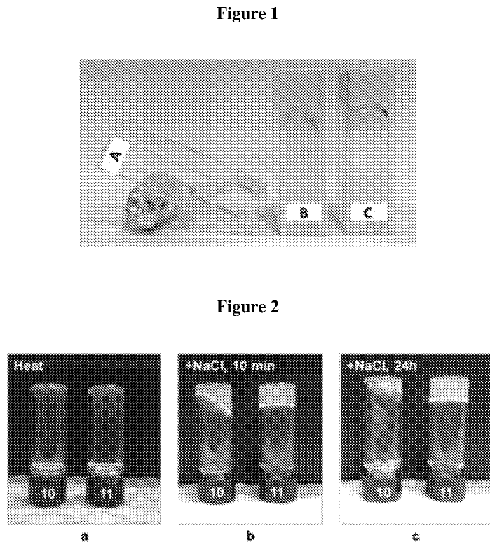

Nitrobenzene-appended short peptide 4 was not soluble in water even with vigorous heating. Therefore, NaOH (2 equivalents) was added to deprotonate the carboxylic acid group at the C-terminus of short peptide 4, generating a sodium carboxylate salt with increased solubility in water. The yellow suspension of short peptide 4 was sonicated for 5 minutes after the addition of alkali, resulting is dissolution and formation of a yellow solution. After standing for 48 hours, the solution became viscous (FIG. 1a), indicating that although self-supporting hydrogel was not observed, self-assembly had occurred in the high pH environment (pH=11).

### Salt Method

The addition of salt (1 equivalent of NaCl) to the basified solution of peptide 4 obtained above, reduced the self-assembly time to 3 hours, and provided self-supporting hydrogels as observed in the vial inversion test (FIG. 1b). The presence of salt is believed to facilitate formation of salt bridges between carboxylate ions, which serve as physical cross-links to provide more robust 3D networks.

### pH Switch

Slightly acidic hydrogels are considered to be more suitable for topical applications. Healthy skin surfaces of most human body parts are slightly acidic with pH values ranging from 4.1 to 5.8. Therefore, the ability of nitrobenzene-appended short peptide 4 to form hydrogels in low pH by using the pH switch method was investigated. The addition of dilute acid induces rapid fiber formation to form a hydrogel. Compared to mineral acids, GdL provides a more homogeneous hydrogel due to its slow hydrolysis rate yet fast dissolution rate in water. The addition of GdL to a high pH solution of short peptide 4 gradually lowered the pH to 5.5. After leaving to stand at room temperature for an hour, a clear yellow self-supporting hydrogel was observed (FIG. 1c), even at a peptide concentration as low as 0.2% w/v. Overall, the nitrobenzene-appended short peptide 4 demonstrated the ability to form a self-assembled hydrogel at either high or low pH, thus allowing it to be used in various applications.

### Critical Gel Concentration

The critical gel concentration was determined through the vial inversion test, as summarised below.

TABLE 2
Hydrogelation conditions of nitrobenzene-appended
hydrogel 4 using addition of salt and pH switch.

| Entry | Trigger | CGC^{a} | Time | pH | Remarks^{b} |
|---|---|---|---|---|---|
| 1 | NaOH | n.a. | 48 h | 11 | VS |
| 2 | NaOH + NaCl | 0.2 | 3 h | 11 | CG |
| 3 | NaOH + GdL | 0.2 | 1 h | 5.2 | CG |

^{a}CGC = Critical gel concentration, denoted as % w/v.
^{b}VS = viscous solution; CG = clear gel

### Cationic Short Peptides

Pre-weighed samples of short peptides 10a-17a, 18, and 19 were prepared in glass vials with 10 mm internal diameter, and Milli-Q water was added to make up concentrations of 1% w/v. The resulting suspensions of short peptides 13, 18, and 19 (as ammonium or guanidinium salts) were slowly warmed until all solid had completely dissolved. GdL (1.5 to 2.0 equivalents) was added to the suspension of short peptides 10a-17a, before heat was applied. To create a high ionic strength environment, 1-5 equivalents of NaCl was added to the viscous solution of 10a-17a to make the total volume up to 1 mL, followed by gentle mixing, and the vial was left to stand without disturbance for 3 hours at room temperature. Subsequently, these vials were inverted and allowed to stand overnight to confirm hydrogel formation.

### Temperature Switch

The temperature switch method is a straightforward way to form self-assembled hydrogels. Short cationic peptides 10-19 containing only phenylalanine unit were subjected to hydrogel formation conditions as follows. After addition of GdL (2.0 equivalents) followed by mild heating, the suspensions of the primary amines 10a and 11a turned clear, which suggested that the primary ammonium gluconate salts 10 and 11 had formed. After cooling over 3 hours, the solutions of 10 and 11 did not show any precipitation.

However, short peptides 10 and 11 failed to form self-supporting hydrogels. A viscous solution was formed which flowed under gravity in the vial inversion test (FIG. 2a). These observations indicated that although a degree of self-assembly had occurred, the strength of intermolecular interactions was not sufficient to allow conversion to the gel phase.

### Salt Method

Addition of salts has been shown to facilitate formation of self-supporting hydrogels in some cases. It was found that upon addition of NaCl (5 equivalents), viscous solutions of short peptides 10 and 11 were transformed into solid-like hydrogels (FIG. 2b). This result was attributed to the fact that at low ionic strength, the electrostatic repulsion between the charged molecules might create a barrier to limit the self-assembly. Ionic solutes, from the salt, could screen the charges and mitigate the electrostatic repulsion to an extent that self-supporting hydrogels could be formed.

However, after being subjected to the vial inversion test for 24 h, only hydrogel 11 (n=3) retained its self-supporting characteristic. Meanwhile, water elapsed slowly from hydrogel 10 (n=2), which was followed by disintegration of the gel, indicating its poor water retention ability (FIG. 2c). This surprising result indicated that for these anthranilamide-based cationic peptides, a chain length of three carbon atoms is preferred in forming a robust hydrogel compared to a chain length of two carbons.

### Critical Gel Concentration

Upon varying the concentrations of short cationic peptide 11 in the vial inversion test, it was found that they formed a stable hydrogel even, surprisingly, at a concentration as low as 0.1% w/v (Table 3).

TABLE 3
Critical gel concentrations (CGCs) and hydrogelation conditions
of anthranilamide-based short cationic peptides 10-14.

| | | Linker [symbol] | | CGC [symbol] | | |
|---|---|---|---|---|---|---|
| Hydrogel [symbol] | Cationic [symbol] ource | length | Triggers | (% [symbol] /V) | remarks | pH |
| 10 | 1° ammonium [symbol] | 2 | GdL, [symbol] heat, [symbol] nd [symbol] NaCl | n.a. | CG → PG | 5.5 |
| 11 | 1° ammonium | 3 | GdL, [symbol] heat, [symbol] nd [symbol] NaCl | 0.1 | CG | 5.5 |
| 12 | 3° ammonium | 3 | GdL, [symbol] heat, [symbol] nd [symbol] NaCl | 0.3 | CG | 5.5 |
| 13 | 4° ammonium | 3 | Heat, [symbol] nd [symbol] NaCl | 0.2 | OG | 6 |
| 14 | Guanidinium | 3 | GdL, [symbol] heat, [symbol] nd [symbol] NaCl | n.a. | OG [symbol] P | 6 |

CG = clear gel;
PG = partial gel;
OG = opaque gel;
P = precipitate

Similar to the primary ammonium compound 11, short cationic peptides bearing tertiary ammonium (12) or quaternary ammonium (13) groups formed hydrogels at low concentrations (0.2% w/v and 0.3% w/v) using a similar trigger of GdL, heat and NaCl. However, the short peptide bearing guanidinium 14 failed to generate a stable structure as the hydrogel collapsed during the vial inversion test.

Introduction of a fluoro substituent on the anthranilamide core did not impede hydrogel formation (Table 4). Interestingly, the fluoro-substituted short peptide 15 showed a lower CGC, possibly due to the electron-withdrawing effect of fluoro on the electronic structure of the anthranilamide-ring. Similarly, the introduction of a chloro substituent (16), a slightly larger electron withdrawing group (EWG), also gave a lower CGC. Surprisingly, the corresponding bromo compound 17 formed an unstable hydrogel which collapsed in the vial inversion test. As the electron-withdrawing groups (i.e. F, Cl, and Br) are not isosteric, these observations indicate that a subtle balance between steric and electronic effects in π-π stacking interactions during the self-assembly is required to obtain a stable hydrogel.

TABLE 4
Critical gel concentration (CGC) and hydrogelation conditions of substituted anthranilamide-based
short cationic peptides 15-19.

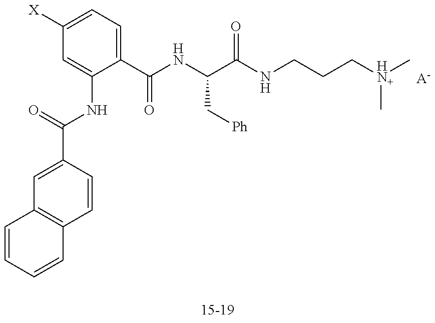

| | | | | CGC | | |
|---|---|---|---|---|---|---|
| | X | A⁻ | Triggers | (% W/V) | Remarks | pH |
| 15 | F | Gdl⁻ | GdL, heat, and NaCl | 0. | OG | 5.5 |
| 16 | Cl | GdL⁻ | GdL, heat, and NaCl | 0.2 | OG | 5.5 |
| 17 | Br | GdL⁻ | GdL, heat, and NaCl | n.a | OG → PG | 5.5 |
| 18 | H | TFA⁻ | heat, and NaCl | 0.3 | CG | 4 |
| 19 | H | Cl⁻ | heat, and NaCl | 0.3 | CG | 5 |

CG = clear gel;
PG = partial gel;
OG = opagque gel

In terms of the counter-anion, changing the gluconate ion to trifluoroacetate (18) or chloride (19) did not significantly affect the CGC of the resulting hydrogels. These peptides successfully formed clear hydrogels via addition of NaCl, with CGC=0.3% w/v.

The anthranilamide-based short cationic peptides 11-19 formed hydrogels with final pH values ranging from 4-6, which is within the range of the surface of healthy human skin, indicating these hydrogels have potential to be used for topical applications.

### Example 3—Characterisation of Hydrogels

### NMR Spectroscopy

NMR samples of nitrobenzene-appended short-peptide 4 were made by dissolving peptide 4 with NaOD (2 equivalents) and an amount of D₂O to make up 0.1 and 0.2% w/v solutions. After the spectra were recorded, either NaCl (1 equivalent) or GdL (2 equivalents) was added to each vial containing 0.2% w/v solution of short peptide 4 and the spectrum was once again recorded.

NMR samples of the monomer were prepared by dissolving 2 mg of cationic short peptide 11 in 650 μL of DMSO d-6. NMR samples of the viscous solution of cationic short peptide 11 were prepared by dissolving compound 11 and GdL in 650 μL of D₂O to make up solutions with 0.1, 0.2, or 0.3% w/v concentration. Subsequently, heat was applied to completely dissolve these samples. After the spectra were recorded, NaCl (5 equivalents) was added to the tube containing short peptide 11 at 0.3% w/v. The resulting viscous solution was vortexed and left for 3 hours to form a hydrogel. The spectrum was once again recorded.

All spectra were obtained on a Bruker Avance III 400 MHz NMR spectrometer, and were processed using the MestreNova software.

### Results

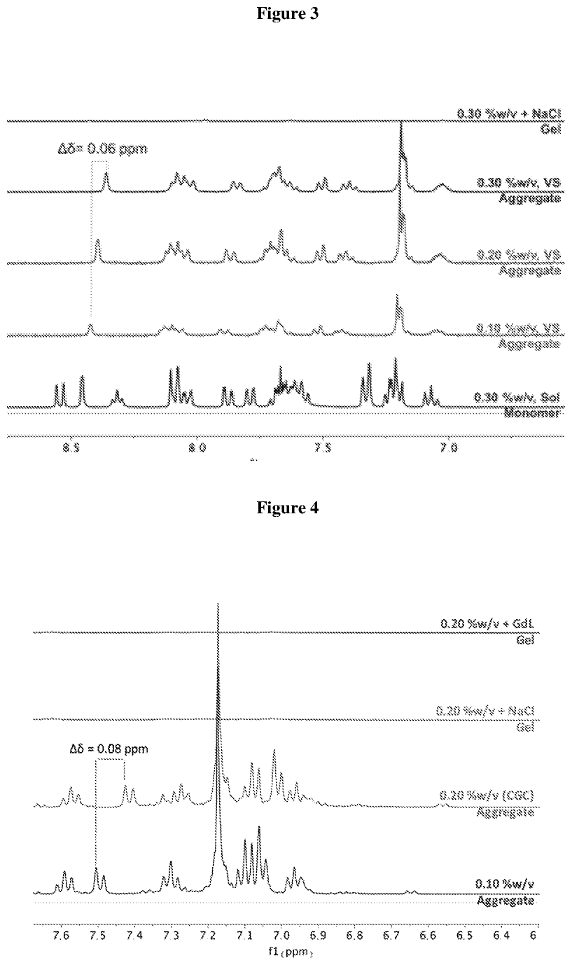

Variations in NMR spectra, such as peak broadening and chemical shift, can indicate the extent of monomer incorporation in self-assembly networks. In solution, peptide monomers show well-resolved NMR signals. Conversely, aggregates such as fibrils frequently show broadening of spectral features due to slower tumbling rates on the NMR time-scale. Therefore, the ¹H NMR spectra of the primary ammonium short peptide 11, as a model compound, in its monomer, aggregate (viscous solution), and hydrogel phases were compared (FIG. 3).

At the same concentration, the ¹H NMR spectrum of the short peptide 11 in its aggregate phase at 0.3% w/v (FIG. 3) displayed comparatively broader NMR features than in its monomer phase. This signified that the short peptide 11 had assembled even before the addition of salt, which is consistent with the observation of a viscous solution in the hydrogelation study. The addition of salt (NaCl) efficiently drives the equilibrium towards the self-assembled state, as evidenced by extreme broadening and loss of ¹H NMR features, hence indicating the formation of a self-supporting hydrogel.

In addition, an up-field shift of the peak (Δδ=0.06 ppm) corresponding to the aryl protons of the capping group was observed upon increasing concentration (0.1% w/v to 0.3% w/v) of compound 11 in its viscous solution (aggregate) phase. This observation is consistent with previous results in our research, which suggested the involvement of the anthranilamide capping group in the self-assembly process through 71-71 stacking interactions.

Addition of salt efficiently drove the equilibrium towards the self-assembled state leading to self-supporting hydrogel formation. The ¹H NMR spectrum of compound 11, recorded after addition of NaCl, showed extreme broadening and loss of ¹H NMR features that indicated complete transformation to their gel phase (FIG. 3). At low ionic strength, the electrostatic repulsion between the charged molecules might create a barrier to limit self-assembly. Ionic solutes, from the salt, could screen the charges and mitigate the electrostatic repulsion to an extent that hydrogels could be formed.

The self-assembly of nitrobenzene-appended short peptide 4 relies mainly on non-covalent interactions, which were also studied using NMR spectroscopy. The ¹H NMR spectra shown in FIG. 4 indicated that the aryl protons of short peptide 4 were involved in driving self-assembly. As the concentration of short peptide 4 increased from 0.10% w/v to 0.2% w/v, an up-field shift of the aromatic protons (Δδ=0.08 ppm) along with some peak broadening were observed, suggesting that π-π stacking interactions play a prominent role in allowing self-assembly to occur. Addition of NaCl or GdL was required to completely transform the solution phase to gel phase, as evidenced by disappearance of ¹H NMR features (FIG. 4).

### UV-Vis Spectroscopy

Concentration dependent UV-Vis spectra of anthranilamide-based short cationic peptide 11 were collected at concentrations ranging from 0.003 mg·mL⁻¹ to 0.050 mg·mL⁻¹. Spectra were collected using an Agilent Cary 60 UV-Vis spectrometer.

### Results

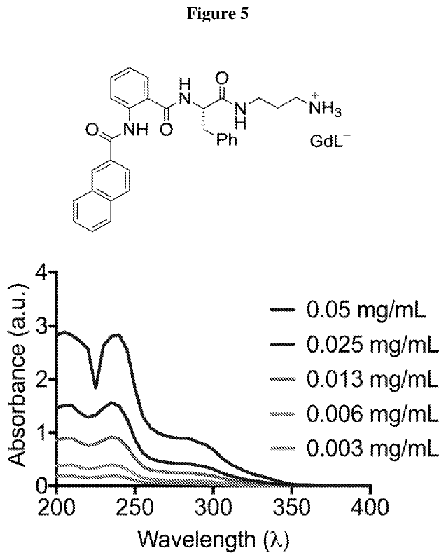

UV-Vis spectroscopy revealed a bathochromic shift from 234 nm to 240 nm and enhancement of a shoulder peak ranging from 250-310 nm, as the concentration of primary ammonium 11, was increased (FIG. 5). A bathochromic shift observed with increasing concentrations corresponds to enhanced π-π stacking and further supports the prominent role of the aromatic capping group in driving the self-assembly process.

### Circular Dichroism (CD) Spectroscopy

### Cationic Short Peptides

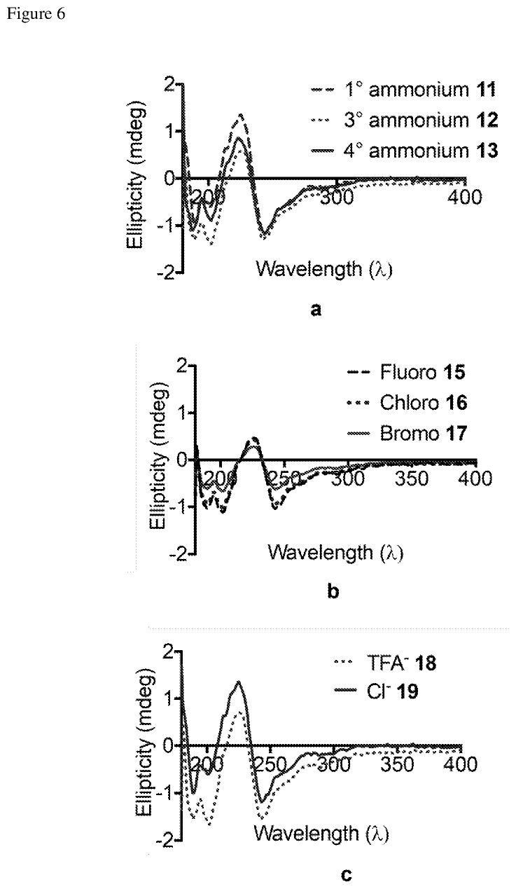

Secondary structures of anthranilamide short cationic peptides were investigated using circular dichroism (CD) spectroscopy (FIG. 6). Hydrogels 11-13, 15, 16, 18, and 19 were prepared at 1% w/v and were diluted 8 times in Mili-Q water before being transferred into a 0.2 mm path length cuvette. The spectra were measured using a ChirascanPlus CD spectrometer (Applied Photophysics, UK) scanning wavelengths of 180-500 nm with a bandwidth of 1 nm, 0.6 s per point, and step of 1 nm. Each experiment was repeated three times and the results were averaged into a single plot value.

When a peptide bond is located in a folded environment, it will generate a CD signal in the far UV region (190-250 nm) that gives information about secondary structures. Peptides and proteins are known to adopt either α-helix, β-sheet, or random coil structures as their common conformational motifs. Each of these secondary structures will give a characteristic CD signal as a result of the π→π* transition (at around 190 nm) and n→π* transition (at 210-220 nm) of the peptide carbonyl groups. By comparison with natural peptides, the secondary structures of anthranilamide-based cationic hydrogels can be determined.

The CD spectra of the hydrogel made from the primary ammonium compound 11 suggested formation of random/disordered coils as indicated by the presence of a negative minimum at around 200 nm and a weak positive maximum at around 220 nm. Similarly, hydrogels made from the tertiary ammonium (12) or quaternary ammonium (13) compounds also exhibited similar CD patterns (FIG. 6a). These observations indicated that the molecular structure of the cationic group did not significantly affect the secondary structures of these short peptides.

The presence of halogen substituents on the anthranilamide core of the first generation of gelators (previous work in our group) had only a very subtle effect on the intensity of CD absorbance observed from dipeptide-based hydrogels. Likewise, introducing halogen substituents and changing the counter-anion did not revamp the secondary structure of the anthranilamide short cationic peptides (FIGS. 6b, 6c).

### NO-Releasing Short Peptides

Initially, 0.8% w/v of hydrogel 4 at high pH (with and without addition of NaCl) and low pH (with addition of GdL) were prepared. These samples were diluted 10× in Milli-Q to obtain the final concentration of 0.08% w/v in water before being transferred into a 0.2 mm path length cuvette. The spectra were measured using a ChirascanPlus CD spectrometer (Applied Photophysics, UK) scanning wavelengths of 180-500 nm with a bandwidth of 1 nm, 0.6 s per point, and step of 1 nm. Each experiment was repeated three times and the results were averaged into a single plot value.

CD spectra were obtained for hydrogels made from short peptide 4 at high pH, low pH, and high ionic strength. CD spectra from hydrogel 4 were compared with the spectra obtained from typical polypeptides to identify their secondary structure. The hydrogel made from nitrobenzene-appended short peptide 4 exhibited characteristics of 0-sheet secondary structure as evidenced by the presence of a strong positive maxima at 197 nm and a subtle negative minima at 230 nm.

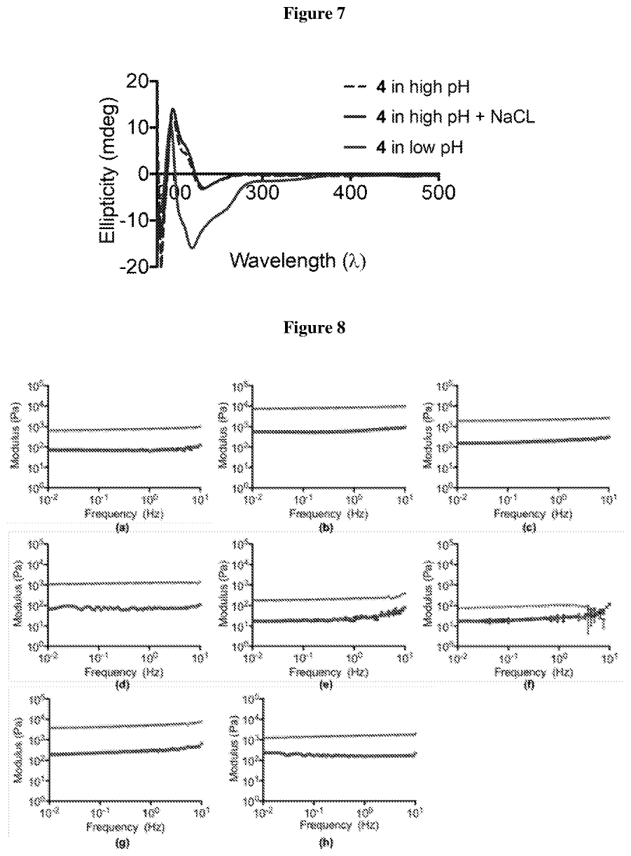

Under all of the different hydrogelation conditions, hydrogel 4 exhibited almost identical CD features, suggesting that the β-sheets conformation was retained (FIG. 7). A dramatic change in CD ellipticity was observed for hydrogel 4 at low pH, which was likely due to the addition of GdL which also absorbs at 218 nm in the CD spectrum.

### Attenuated Total Reflectance Fourier Transform Infrared (ATR-FTIR) Spectroscopy

To confirm the secondary structure of the hydrogels observed by CD spectroscopy, ATR-FTIR was performed on the D₂O gels and xerogels (air-dried hydrogels) of anthranilamide-based short cationic peptides.

ATR-FTIR spectra were obtained using a Spectrum 100 FTIR spectrometer (PerkinElmer, USA) fitted with a 1 mm diamond-ZnSe crystal. Xerogels were made in situ by applying nitrogen flow to one drop of the pre-formed hydrogels (2% w/v) which was placed on the ATR-crystal. The spectra of xerogels 11-13, 15, 16, 18, and 19 were recorded from 4000 cm⁻¹ to 650 cm⁻¹ with a 4 cm⁻¹ resolution and 4 scans. The spectra of D₂O gels were measured by applying two drops of pre-formed gels at 2% w/v on the ATR crystal which was then recorded from 4000 cm⁻¹ to 650 cm⁻¹ with a 4 cm⁻¹ resolution and 4 scans.

All of the D₂O gels and xerogels tested exhibited peaks at 1645±4.0 cm⁻¹ and 1648±2.0 cm⁻¹ (Table 5), respectively, which indicated the presence of random or disordered coil secondary structures.

TABLE 5
ATR-FTIR signals of D₂O gels and xerogels of anthranilamide-based cationic short peptides.

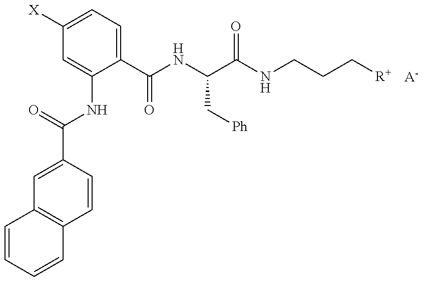

| | D₂O gels | Xerogels |
|---|---|---|
| | (cm⁻¹) | (cm⁻¹) |
| 1° ammonium 11 | 1641 | 1648 |
| 3′ ammonium 12 | 1647 | 1647 |
| 4′ ammonium 13 | 1643 | 1647 |
| Fluoro 15 | 1645 | 1648 |
| Chloro 16 | 1642 | 1649 |
| Bromo 17 | 1648 | 1650 |
| TFA⁻ 18 | 1641 | 1647 |
| Cl⁻ 19 | 1647 | 1647 |

The secondary structure of short peptide 4 was also confirmed by FTIR analysis on the native gel and xerogel (air-dried hydrogel). The amide I region (1700-1600 cm⁻¹) contains the most sensitive vibrational bands of a peptide backbone, and therefore provides crucial information of secondary conformation. Peaks at 1635 cm⁻¹ and 1631 cm⁻¹, which correspond to β-sheet secondary structures, were found in native and xerogel states of short peptide 4. This result further supports the CD spectroscopy data which pointed to the formation of β-sheets as the predominant secondary structure in both native and xerogel state.

### Rheology

For topical antibacterial applications, AMPs have been mixed with hypromellose (a gel base), to obtain a more viscoelastic formulation. In contrast, the self-assembled properties of anthranilamide-capped ultra-short cationic peptide mimics of the present invention allows formation of hydrogels with excellent viscoelastic properties, without the presence of any additive.

Rheology was used to precisely determine the mechanical properties of the hydrogels. Rheology describes the study of the flow and deformation of materials, including viscoelastic materials such as hydrogels. The frequency sweep test (FST) and strain sweep test (SST) are the most basic experiments that can provide data into the mechanical properties such as stability, rigidity, and malleability of soft materials. Data obtained from FST and SST can also be used to define suitable applications of the generated hydrogels.

### Frequency Sweep Test (FST) Analysis

### Cationic Short Peptides

The mechanical properties of anthranilamide-based cationic hydrogels were assessed using an Anton Paar MCR 302 rheometer with a 25 mm stainless parallel plate configuration.

Initially, 1 mL of hydrogels were prepared from short peptides 11-13 and 15-19 in glass vials at concentration of 1% w/v. These vials were warmed using a heat gun to transform the hydrogel to the solution phase. Subsequently, 560 μL of the resulting solutions were cast onto the rheometer plate. The other plate was lowered to the measuring position (1 nm) and the hydrogel was allowed to stand for 3 hours for the gel to form. The FST was conducted at fixed strain of 0.1% and frequency ranging from 10 Hz to 0.01 Hz. Meanwhile, the strain sweep test (SST) was performed at a frequency of 1 Hz using 0.1% strain to 100% strain. The rheology data were shown as the average of three repeats for each data point.

### NO-Releasing Peptides

The mechanical properties of hydrogels 4 (formed via addition of NaCl or GdL) before and after blue light irradiation were also assessed using an Anton Paar MCR 302 rheometer with a 25 mm stainless parallel plate configuration. Prior to the hydrogel formation, 560 μL of solution phase of short peptide 4 at 1% w/v were transferred onto a rheometer plate. Meanwhile, hydrogel 4, which had been exposed to blue light, was warmed using a heat gun, to provide its solution phase. Subsequently, 560 μL of this solution phase was cast onto the rheometer plate. The other plate was lowered to the measuring position (1 nm) and the hydrogel was allowed to stand for 3 hours for the gel to form. The frequency sweep test (FST) was conducted at fixed strain of 0.1% and frequency ranging from 10 Hz to 0.01 Hz. Meanwhile, the SST was performed at a frequency of 1 Hz using 0.1% strain to 100% strain. In addition, the temperature sweep test was conducted to obtain transition glass (Tg) using fixed frequency of 1 Hz and fixed strain of 0.1% with temperature ramping from 25° C. to 90° C. The rheology data were shown as the average of three repeats for each data point.

### Results

In the FST, samples were measured over a range of oscillation frequencies (0.1-10 rad/s) at fixed strain, temperature, and oscillation amplitude. Higher frequencies simulate fast motion on a short time-scale, while low frequencies simulate slow motion on a long time-scale, or rest. All of the hydrogels made from the anthranilamide-based short cationic peptides, which passed the vial inversion test, were subjected to the FST using a rheometer to precisely assess their mechanical properties.

The majority of the short cationic peptides (except bromo compound 17) demonstrated significantly higher modulus storage (G′) compared to their modulus loss (G″) at all frequencies (FIG. 8). As noted above, G′ represents the elastic proportion (solid-state), whereas G″ represents the viscous proportion (liquid-state) of a viscoelastic material. To be classified as a stable hydrogel, a material should exhibit a G′ value at least an order magnitude higher than its G″ value in FST. Therefore, these results further confirm the structural stability of the hydrogels.

In addition, modulus storage (G′) is often used as a measure of the mechanical rigidity (stiffness) of a hydrogel. Higher G′ values are frequently ascribed to more robust hydrogels. Additionally, the mechanical rigidity of these hydrogels can be modulated by carefully altering the molecular design of the hydrogelators, particularly through variation of the cationic group, substituents on the benzene ring of the anthranilamide core, and the counter anion. Varying the terminal cationic group of the anthranilamide-based short cationic peptides resulted in notable changes in the G′ value of the resulting hydrogels (Table 6). Interestingly, changing the cationic group from primary ammonium 11 to tertiary ammonium 12 and quaternary ammonium 13 increased the G′ value from 0.83 kPa to 8.63 kPa and 2.28 kPa, respectively. The presence of a methyl group, in hydrogels made from tertiary ammonium 12 and quaternary ammonium 13 was thought to decrease the flexibility of these short peptides, which presumably leads to the formation of more stable and robust hydrogels.

TABLE 6
Modulus storage (G′) and modulus loss (G″) obtained from frequency
sweep test (FST) performed on hydrogels made from short peptides
11-13, bearing a variety of cationic groups.

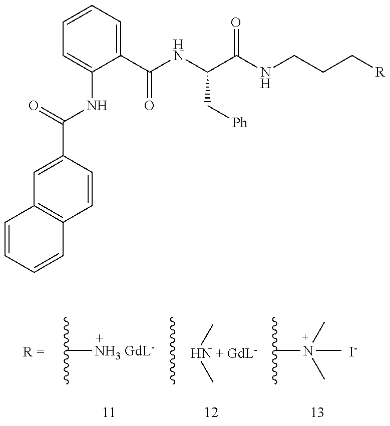

| | G′ | G″ | G′/G″ |
|---|---|---|---|
| Hydrogelators | (kPa) | (kPa) | ratio |
| 1° ammonium 11 | 0.83 | 0.07 | 11.9 |
| 3° ammonium 12 | 8.63 | 0.55 | 15.7 |
| 4° ammonium 13 | 2.28 | 0.21 | 10.9 |

Furthermore, the presence of electron-withdrawing substituents (i.e. F, Cl, Br) on the anthranilamide core of these short peptides affected the overall rheological properties of the resulting hydrogels. The mechanical strength/rigidity gradually decreased as larger EWGs were introduced (G′ of F>Cl>Br). Surprisingly, changing a hydrogen for fluorine atom on the anthranilamide moiety resulted in a decrease of G′ value from 8.63 kPa to 1.20 kPa (compare compound 12, Table 6 and compound 15, Table 7):

TABLE 7
Modulus storage (G′) and modulus loss (G″) of hydrogels 15-19 obtained from the frequency
sweep test performed using a rheometer.

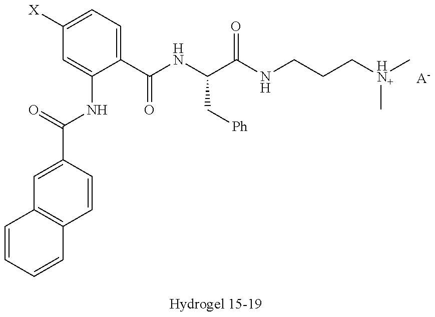

| | | G′ | G″ | G′/G″ |
|---|---|---|---|---|
| X | A⁻ | (kPa) | (kPa) | ratio |
| F 15 | GdL⁻ | 1.20 | 0.06 | 20 |
| Cl 16 | GdL⁻ | 0.53 | 0.05 | 10.6 |
| Br 17* | GdL⁻ | 0.08 | 0.02 | 4.0 |
| H 18 | TFA⁻ | 5.12 | 0.31 | 16.5 |
| H 19 | Cl⁻ | 1.61 | 0.16 | 10.1 |

*Collapsed (G′ = G″) in high frequency (F ≥ 5 Hz)

A further decrease in G′ value (0.53 kPa) was observed for hydrogel 16, bearing chloro as a substituent. Nevertheless, both fluoro 15 and chloro 16 demonstrated prominent characteristics of stable hydrogels, as G′ and G″ were at least an order of magnitude apart (FIGS. 8d and 8e). The incorporation of bromine led to a further decrease in the G′ value to 0.08 kPa. Moreover, the hydrogel made from short peptide 17 also lost its gel characteristic when a high frequency (≥5 Hz) was applied, as indicated by G′ falling below G″ (FIG. 8f), suggesting that it is a metastable hydrogel.

Changing the counter-anion from gluconate (12) to trifluoroacetate (18) resulted in a subtle change in the rigidity of the hydrogel. However, the hydrogel made with a chloride counter-anion (19) exhibited a notable decrease of G′ value (Table 7), presumably due to the absence of H-bonding capability of this anion.

Overall, these observations show that electronegative and steric effects of substituents can be used to fine-tune the mechanical properties of these hydrogels.

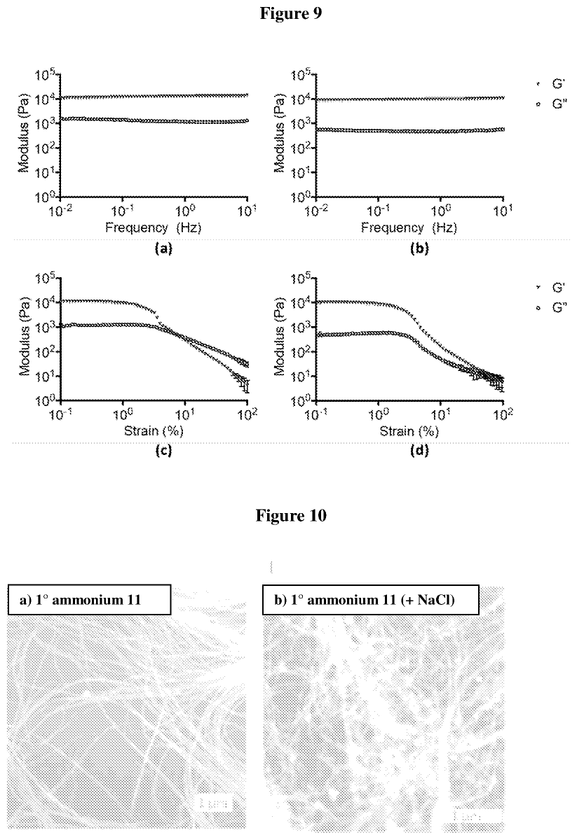

FST results on short peptide 4 at both low and high pH (in the presence of salt), showed characteristics of stable hydrogels as indicated by their modulus storage (G′) values which are at least an order of magnitude higher than their modulus loss values (G″) (FIGS. 9a-b).

As noted above, the G′ value obtained from FST is often used as a measure of the rigidity of a hydrogel. Hydrogels made from short peptide 4 at high and low pH showed G′ values of 13.2 kPa and 10.2 kPa, respectively (Table 8), which are considered to be suitable values for topical applications.

TABLE 8
Mechanical properties of nitrobenzene-
appended hydrogel 4 at high and low pH.

| | G′ | G″ | | |
|---|---|---|---|---|
| pH | (kPa) | (kPa) | G′/G″ | % strain |
| 11 | 13.2 | 1.1 | 12 | 1.2 |
| 5.5-6 | 10.2 | 0.6 | 17 | 1.4 |

### Strain Sweep Test (SST) Analysis

### Results

To observe the structural decay in anthranilamide-based cationic hydrogels, the SST was performed by imposing a non-linear strain. SST provides information about the viscoelastic properties of a hydrogel through determination of its linear viscoelastic region (LVER). The LVER indicates the range where increase of strain level does not affect the mechanical properties of a hydrogel. Here, the LVER reported for each hydrogel represents the deviation of storage modulus (G′) from linearity, known as yield point (γ_{y}). Yield points have been reported to reflect a point where the rigidity of a hydrogel starts to weaken.

Despite having different cationic groups, hydrogels made from peptides 11-13 displayed similar LVER (˜1%), as summarised in Table 9. The halogen-substituted fluoro (15) and chloro (16) hydrogels show a greater LVER of 2.01% and 1.66%, respectively.

In contrast, the bromo (17) hydrogel resulted in a narrower LVER (0.16%). These observations suggest that the substituent on the 5-position of the anthranilamide core could be varied to finely tune the properties of the resulting hydrogels. In addition, varying the counter-anion (i.e. trifluoroacetate and chloride) did not change the LVER.

TABLE 9
Linear viscoelastic region (LVER) of hydrogels 11-13, 15-19 obtained
via strain sweep test (SST).

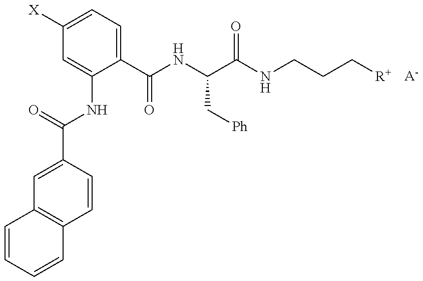

| Hydrogel | LVER (%) |
|---|---|
| 1° ammonium 11 | 0.98 ± 0.05 |
| 3° ammonium 12 | 0.98 ± 0.02 |
| 4° ammonium 13 | 1.10 ± 0.04 |
| Fluoro 15 | 2.01 ± 0.06 |
| Chloro 16 | 1.66 ± 0.10 |
| Bromo 17 | 0.16 ± 0.01 |
| TFA⁻ 18 | 0.98 ± 0.02 |
| Cl⁻ 19 | 1.00 ± 0.01 |

Overall, for the cationic short peptide hydrogels, the FST and SST observations indicated that the mechanical properties, in particular the rigidity, can be modulated by carefully altering the molecular design of the hydrogelators through the choice of cationic groups, substituents on the benzene ring of the anthranilamide core, or the counter-anion.

The NO-releasing short peptide was also analysed by SST. Hydrogels made from short peptide 4 exhibited very similar LVERs at both high and low pH (FIGS. 9c-d). This indicated that hydrogels formed from peptide 4 possessed similar mechanical properties, regardless of the final pH.

### Atomic Force Microscopy (AFM)

To gain some insight into the relationship between the supramolecular network of a hydrogel and its mechanical properties, AFM was employed.

Hydrogels of NO-releasing short peptide 4 (1 drop), at high pH, low pH, and after irradiation, were cast onto a mica substrate. Hydrogels of cationic short peptides 11-13 and 15-19 were prepared at their CGC, 2× below, and 4× below CGC in glass vials. Prior to gelation, one drop of the cationic short peptide solutions was cast onto a mica substrate. Using a glass slide, each droplet was carefully spread and was left to dry overnight before imaging. Imaging was performed using a Bruker Multimode 8 Atomic Force Microscope in Scanasyst Air (PeakForce Tappings) mode, which is based on tapping mode AFM. To prevent damage of soft samples, the imaging parameters were constantly optimized through the force curves that were collected. Bruker Scanasyst-Air probes were used, with a spring constant of 0.4-0.8 N·m⁻¹ and a tip radius of 2 nm.

To gain some insight on the mechanism of antibacterial action, the released solutions from hydrogel made of primary ammonium 11 were directly cast onto a mica substrate. In addition, respective amounts of peptide mimic 11 and GdL were dissolved in DMSO-water (5:95) which was then diluted to obtain a concentration of 221 μM before being cast on a mica substrate. After gently spreading using a glass slide and left to dry overnight, these samples were imaged using a similar manner as describe above.

### Results

All of the xerogels made from peptide mimics 11-13 and 15-19 exhibited nano fibrous networks with a lot of junction zones which provided space responsible for immobilizing water molecules leading to hydrogel formation (FIGS. 10-12). Variation of the chemical groups resulted in differences in fiber size (diameter) but did not dramatically alter the morphology of the resulting hydrogels as discussed below. A change in the fiber diameter could affect the mechanical properties of the resulting hydrogel. For instance, thicker fibers, hence smaller pores, could result in restricted molecule displacement which may correspond with formation of a more robust hydrogel.

The fiber morphology of the hydrogel made from the primary ammonium compound 11 significantly evolved upon the addition of salt. In the absence of salt, a viscous solution of short peptide 11 displayed self-assembly into fibers, with diameter of 74±6 nm at concentrations as low as 0.05% w/v, however, bundling and junction zones were not observed (FIG. 10a). This observation shows that the addition of salt to the viscous solution of primary ammonium 11 promotes fiber aggregation.

Upon addition of salt, these fibers bundled together to form larger fiber bundles with diameter of 115±5 nm (FIG. 10b). This observation showed that the addition of salt to the viscous solution of primary ammonium 11 promotes fiber aggregation, which could be critical in forming a self-supporting hydrogel.

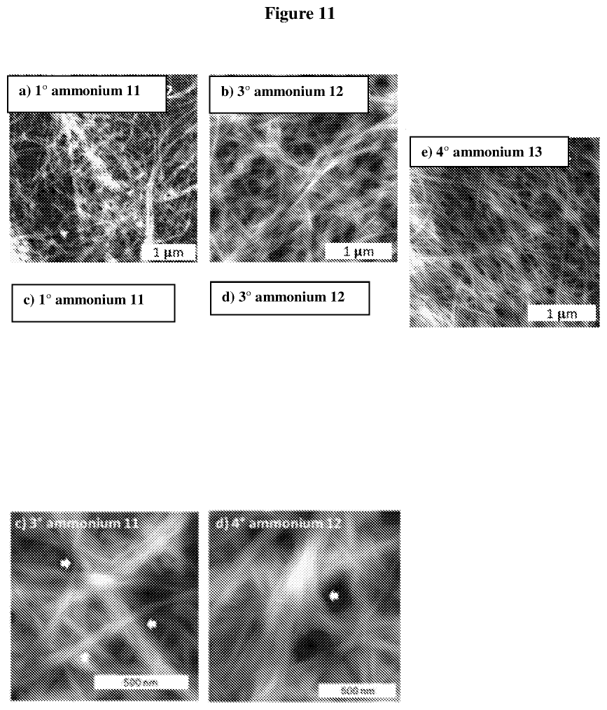

Compared to the primary ammonium 11, larger bundles were observed from the tertiary ammonium 12 (139±10 nm) and quaternary ammonium 13 (148±16 nm) compounds (FIG. 11). These results were consistent with the mechanical properties observed in rheology. The formation of thicker bundles, hence smaller pores, result in restricted molecule displacement which corresponds to the more robust characteristics observed in mechanical tests.

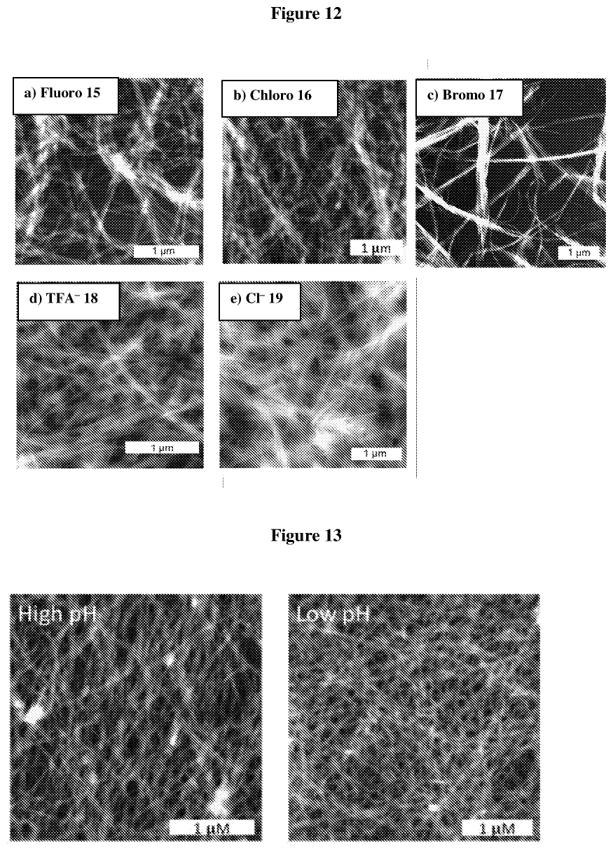

Hydrogels bearing fluoro 15 (FIG. 12a) and chloro 16 (FIG. 12b) as substituents exhibited similar fiber morphology to the unsubstituted hydrogel 11. However, the fluoro 15 and chloro 16 hydrogels showed formation of slightly thinner bundles with diameter of 115±10 nm and 99±6 nm, respectively. On the other hand, the hydrogel of the bromo compound 17 exhibited rather different morphology, with inflexible fibers with diameter of 101±6 nm being observed (FIG. 12c). Even though this hydrogel was imaged at higher concentration (0.10% w/v), junction zones were barely observed which presumably contributed to their brittleness observed in rheology.

Moreover, the fiber morphology of the hydrogel made from the tertiary ammonium 12 compound was found to be insensitive to the identity of the counter-anion. Aside from diameter, fiber morphology observed from short peptides bearing trifluoroacetate 18 (104±4 nm) (FIG. 12d) and chloride 19 (87±6 nm) (FIG. 12e) were indistinguishable to each other as well as to the corresponding hydrogel 12 bearing gluconate ion.

Despite their final pH, AFM showed that the nitrobenzene-appended hydrogels 4, which formed in high and low pH, displayed flexible fibers with indistinguishable morphology and diameters of 40±3 nm and 43±5 nm respectively (FIG. 13). In addition, these individual fibers were bundled together to form larger fibers with diameters of 104±4 nm, which accounted for their stable and robust (G′=˜10 kPa) characteristics observed in rheology.

Previous studies have reported that hydrogels which formed in high pH (pH>8) often exhibited toxicity issues (Wang et al., Int. J. Pharm. 2010, 389, 130). Therefore, the hydrogel 4 which was formed at pH 5.5-6 was chosen to be subjected to cytotoxicity, release, and antibacterial studies.

### Example 4—Antibacterial Activity of Hydrogels

### Cationic Short Peptides

Toxicity Against S. aureus and E. coli

The antibacterial activity of the anthranilamide-based cationic hydrogels 11-13 and 15-19 was investigated in vitro against S. aureus. The antibacterial activity of anthranilamide-based cationic hydrogel 11 was also investigated in vitro against E. coli.

First, a single colony of S. aureus 38 or E. coli K12 was grown in Luria-Bertani (LB) broth medium at 37° C. overnight. The resulting bacteria were harvested, via centrifugation, to obtain a bacteria pellet which was re-suspended in the same volume of LB medium, twice. The optical density (OD) value of the resulting culture was adjusted to 0.1 at 600 nm in LB (108 cfu·mL⁻¹). This bacteria solution was then further adjusted to 3×10⁴ cfu mL⁻¹. (CFU or cfu=colony-forming unit). Bacteria solutions (1 mL), which contain either 10⁸ CFUs/mL or 3×10⁴ CFUs/mL, were gently added to hydrogel 11 at 1% w/v with total volume of 1 mL. Anthranilamide-based cationic hydrogels 11-13 and 15-19 (1 mL) were prepared at 1% w/v in glass vials as described above. On top of these pre-formed hydrogels, 1 mL of bacteria solution was carefully cast. In addition, the acetyl hydrogel, which has been reported to be inactive, was used as a comparison. As a negative control, a bacterial solution in the absence of hydrogel was used. After being incubated at 37° C. for 18 hours, 100 μL of bacteria solution in each vial was taken and subjected to serial dilution using phosphate buffer solution (PBS). 20 μL of each dilution were carefully transferred into nutrient agar plates and incubated for another 18 hours. The following day, bacterial growth inhibition was quantified using the viable count method. The experiment was performed twice in triplicate and multiple sample comparison was performed using one-way ANOVA at p<0.05.

Bromo hydrogel 17 could not be tested because it disintegrated as soon as bacteria culture (1 mL) was cast on its surface. Due to its brittle characteristic, this hydrogel was not investigated further.

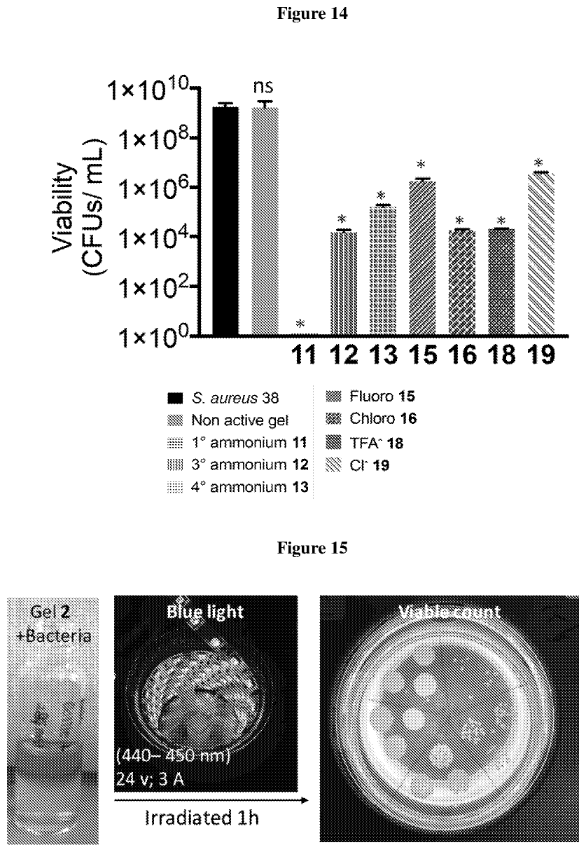

The hydrogels tested all showed significant bacterial reduction, by at least three orders of magnitude compared to the control (FIG. 14). Surprisingly, the primary ammonium 11 compound was found to be the most active hydrogel and exhibited 9.0 Log₁₀ bacterial reduction. In addition, the tertiary ammonium 12 and quaternary ammonium 13 hydrogels exhibited 5.1 Log₁₀ and 4.1 Log₁₀ bacterial reduction respectively. These observations suggest that not only the charge, but also the molecular structure of the cationic groups could affect the antibacterial activities of the resulting hydrogels. Increasing alkyl substitution on the ammonium ion (11→12→13) led to decreasing antibacterial activity of the resulting hydrogels, presumably due to the decrease in membrane binding capability.

Introducing a halogen atom as a substituent on the anthranilamide core, or varying the counter anion did not significantly improve the antibacterial activity of the parent hydrogel 12. The fluoro 15 and chloro 16 hydrogels showed 3.1 Log₁₀ and 5.0 Log₁₀ bacterial reduction, respectively (FIG. 14). Meanwhile, hydrogels 18 (TFA⁻) and 19 (Cl⁻), showed 4.9 Log₁₀ and 3.0 Log₁₀ bacteria reduction respectively.

Hydrogel 11 was also challenged against Gram-negative E. coli bacteria, which is associated with skin and soft tissue infections (SSTI). The hydrogel made from primary ammonium 11 showed notable (6.4 Log 10) bacteria reduction against E. coli (FIG. 23). The difference in antibacterial activity, compared to the Gram positive bacteria, was presumably due to the presence of an additional outer bilayer membrane consisting of lipopolysaccharide and phospholipid in Gram-negative bacteria.

### NO-Releasing Short Peptides

Aside from the common causative bacteria (i.e. Staphylococcus aureus and aerobic Streptococci), E. coli has also been associated with skin and soft tissue infections (SSTI). In addition, E. coli is the least susceptible to blue light irradiation, compared to other SSTI-causative bacteria. Therefore, the efficacy of NO release from nitrobenzene-appended hydrogel 4 was evaluated against E. coli K12.

The antibacterial activity of NO released from hydrogel 4 was investigated in vitro. Initially, a single colony of E. coli K12 was grown in LB broth medium (Sigma-Aldrich) at 37° C. overnight. The resulting bacteria were harvested via centrifugation to obtain a bacteria pallet which was re-suspended in the same volume of LB, twice. The optical density value of the resulting culture was adjusted to 0.1 at 600 nm (108 cfu·mL⁻¹). This bacteria solution was then further adjusted to 3×10⁴ cfu·mL⁻¹.

Hydrogel 4 was prepared at 1% w/v in two different vials. The first vial (vial a) contained hydrogel 4 which will be exposed to blue light. Meanwhile vial b contained hydrogel 4, which will be covered using aluminium foil during the entire experiment. Furthermore, on top of these pre-formed hydrogels, 1 mL of bacteria solution was carefully cast. A vial c that contained only 1 mL of bacteria solution was also prepared. Subsequently, vial a and vial c were irradiated using blue light (440-450 nm). As a negative control, a bacteria solution in the absence of hydrogel and blue light was used in this experiment. After 2 hours, 100 μL of bacteria solution in each vial was taken and subjected to serial dilution using PBS. 20 μL of each dilution were carefully transferred into nutrient agar plates and incubated for another 18 hours. The following day, bacterial growth inhibition were quantified using viable count method. The experiment was performed twice in triplicate and multiple sample comparison was performed using one-way ANOVA at p<0.05.

Bacterial culture (1 mL containing 3×10⁴ CFU) was carefully placed on top of hydrogel 4, followed by irradiation of blue light for 1 hour. The viability of bacteria after being exposed to blue light was calculated via the viable count method as illustrated in FIG. 15.

Hydrogel 4, by itself, might exert activity against E. coli. Therefore, the antibacterial activity of hydrogel 4 in the absence of blue light was also determined in a control experiment. In addition, to avoid bias from the bactericidal effect of blue light, a vial containing bacterial culture was exposed to blue light for the same amount of time in the absence of hydrogel 4.

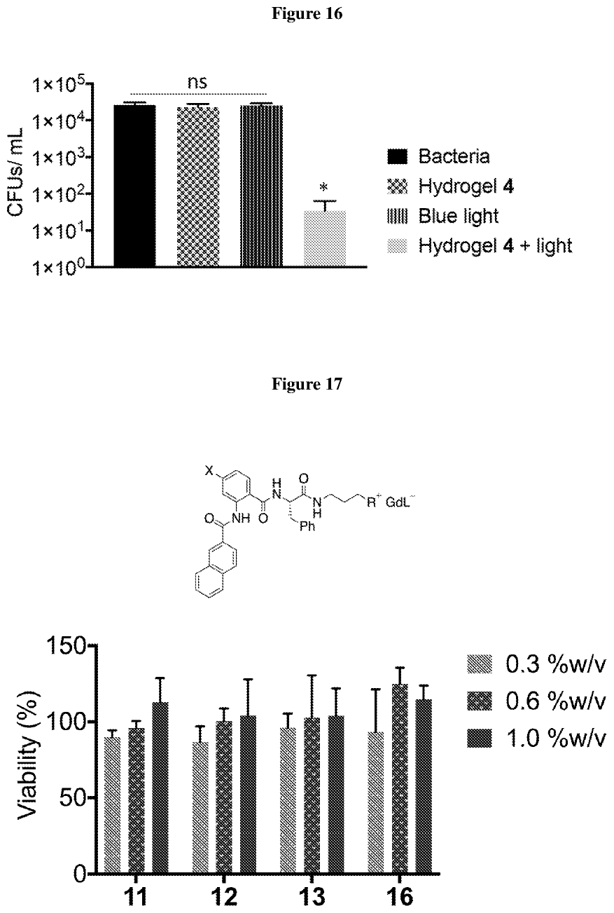

Blue light irradiation of hydrogel 4 for 1 hour significantly reduced the bacterial number from 3×10⁴ CFUs to 34 CFUs (˜3 Log reduction) (FIG. 16). However, although the concentration of NO released was expected to kill 100% of E. coli, a few bacteria were able to survive. This deviation might be due to the use of broth in the antibacterial assay, whereas the Griess assay used Milli-Q water. Broth contains a more complex mixture of substances, which presumably allows possible side reactions with NO thus decreasing the antibacterial efficacy of NO.

Conversely, there was no significant bacteria reduction observed for the irradiated culture in the absence of hydrogel 4. This indicated there was not enough irradiation time or energy from the blue light used in this experiment to exert antibacterial activity against E. coli. In addition, the fact that no bacterial reduction was observed for hydrogel 4 without light irradiation further emphasised that the release of NO was strictly light-dependent, while also indicating that hydrogel 4 showed no inherent antibacterial activity of its own.

### Example 5—Toxicity Against HEK 293T Cells

To be an ideal candidate for a topical antibacterial, a hydrogel not only needs to exhibit excellent antibacterial properties, but should also have low toxicity against normal cells. Therefore, the cytotoxicity of selected hydrogels was examined against HEK 293T cells.

Cytotoxicity measurements were performed using an Alamar Blue colorimetric assay. Each experiment was repeated at least three times. Cells were passaged using standard cell culture procedures. Cells were detached with trypsin and centrifuged (1000 rpm for 3 min). The supernatant was removed and the cells re-suspended in Dulbecco's Modified Eagle Medium (DMEM) at a concentration of 100,000 cells per mL. Cells were seeded at a concentration of 6,000 cells per well. For cytotoxicity measurements, 100 mL of hydrogel made from short-peptide 4 (at 1% w/v and 2% w/v) and short peptides 11, 12, 13, and 16 (at 0.3, 0.6, and 1.0% w/v) were added in triplicate to a 96-well plate and allowed to set overnight. Surrounding wells were supplemented with water to ensure hydration of the gels. Gels were then incubated for 24 h with DMEM. Cells were seeded atop the hydrogels and incubated for 24 hours, before 10 mL of Alamar Blue was added to the wells, followed by further incubation for 4 h. Control wells included cell-free gels, no hydrogels and a negative control of 15% (v/v) DMSO. The absorbances at 570 nm and 596 nm were recorded using a BioRad Benchmark plate reader.

### Results

The presence of cationic groups was expected to decrease hydrophobicity leading to lower toxicity towards normal cells. The primary ammonium 11, tertiary ammonium 12, quaternary ammonium 13 and chloro-substituted 16 hydrogels all showed good mechanical properties and were also the four most potent hydrogels in terms of antibacterial activity. Therefore, these hydrogels were chosen to be examined against HEK 293T cells, as model mammalian cells, in vitro.

Cationic hydrogels 11, 12, 13 and 16 exhibited minimal toxicity against HEK 293T cells, with no significant variation with increased concentrations (FIG. 17). More than 80% cell viability was observed for these hydrogels, indicating they would not be toxic to normal cells. These results suggest that the presence of cationic groups in anthranilamide-based hydrogels significantly decreases the toxicity against normal cells while maintaining their antibacterial function, and potentially allows them to be promising candidates for antibacterial biomaterials.

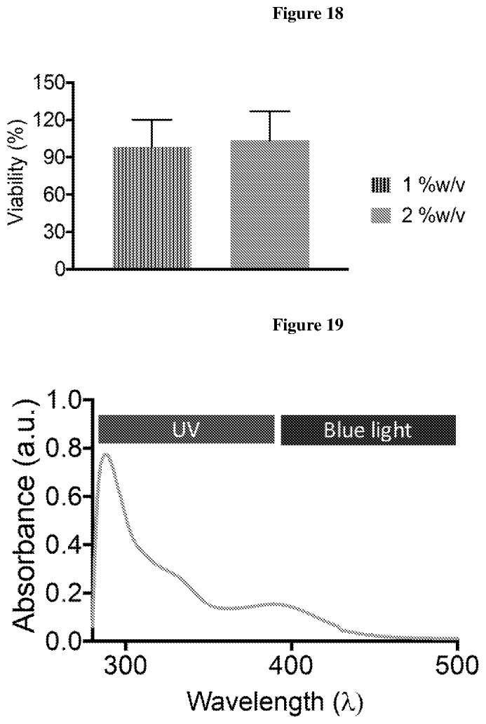

The cytotoxicity of the nitrobenzene-appended short peptide 4 against HEK 293T cells was also investigated in vitro. In the presence of hydrogel 4 at 1% w/v, which was the concentration used in the NO release study, HEK 293T cells showed more than 90% cell viability compared to the control (FIG. 18). A similar result was observed at higher concentration (2% w/v), thus indicating that hydrogels made from nitrobenzene-appended short peptide 4 also exhibited very low toxicity against HEK 293T cells.

### Example 6—NO Release from Hydrogel Made from Nitrobenzene-Appended Short Peptide 4

Nitrobenzene appended short peptide 4 (2.8 mg) was prepared at a concentration of 0.02% w/v; GdL (2 equivalents) was added to adjust the pH prior to analysis by UV-Vis spectroscopy.

Nitrobenzene derivatives can undergo photocleavage to produce NO radicals in the presence of light. Similar to the common absorbance for NO chromophores, the nitrobenzene part of the hydrogel 4 exhibited a broad peak ranging from 350 to 460 nm, which was centred at around 398 nm (FIG. 19).

NO release from hydrogel 4 was quantified using the Griess reagent, which is an indirect method that measures collected nitrite as the primary degradation product of NO. Nitrobenzene-appended anthranilamide-based hydrogel 4 was pre-formed in an aluminium covered vial at 1% w/v (1 mL). Milli-Q water (1 mL) was carefully cast on top of the hydrogel, which was subsequently exposed to UV or blue light irradiation. At certain time points (0, 5, 15, 30, 60, and 120 min), the entire upper solutions were carefully removed and allowed to react with Griess reagent, according to the indication of the NO assay kit (Promega), to provide an azo compound that is responsible for providing the colorimetric assay. Subsequently, 1 mL of fresh Milli-Q water was immediately added to the top of hydrogel 4 for continuing the release.

Due to the strong absorbance of hydrogel 4 in the UV range, NO release was triggered by using a UV lamp (λ=356 nm). The same volume of Milli-Q water was placed on top of hydrogel 4, which was irradiated with UV light for 2 hours. The entire solution, on top of the hydrogel, was removed and replaced with the same amount of Milli-Q water periodically. The resulting solutions were reacted with Griess reagent. Any NO generated during the experiment would react with oxygen to generate nitrite ion (NO₂⁻). The subsequent reaction between NO₂⁻ with Griess reagent, which contains sulfanilamide and N-(1-naphthyl)ethylenediamine (NED), will form a bright pink azo-dye compound that can be quantified using UV-Vis spectroscopy, as shown in the below scheme:

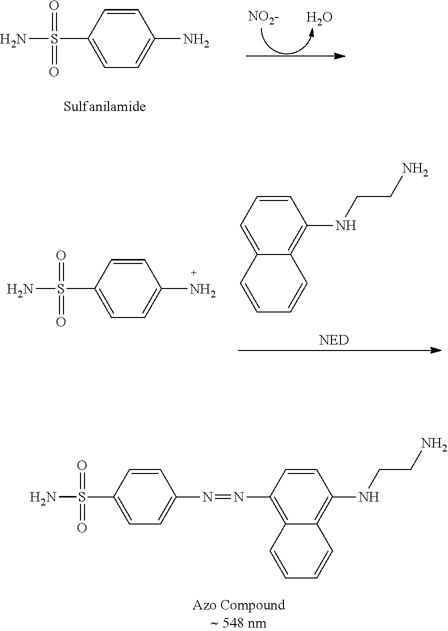

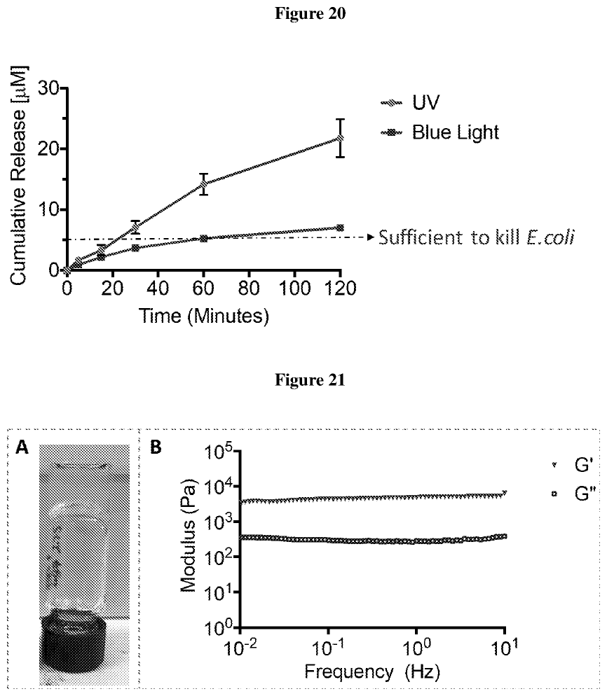

Under UV light irradiation, the nitrobenzene-appended hydrogel 4 from 1% w/v exhibited continuous release of NO over 2 hours (FIG. 20, circles). A concentration of NO of 5 μM is sufficient to kill E. coli. Even only after 5 minutes, 1 μM NO was detected. Irradiation for 30 minutes generated 7 μM of NO. At 2 hours, 61% of NO was recovered and therefore suggest the potential of hydrogel 4 to be used for NO delivery.

The use of UV as a trigger to generate NO might cause phototoxicity. UV radiation, specifically UV A (295-400 nm), can damage human skin through formation the formation of lesions, which can progress to skin cancer. Therefore, to avoid the side effects of UV irradiation, blue light was employed as an alternative trigger to generate NO from hydrogel 4. Short term application of blue light (420-455 nm) has been reported to be dermatologically safe, as indications of DNA damage were not observed. Although NO release was observed, blue light irradiation exhibited slower NO release compared to UV light irradiation (FIG. 20, squares). A longer irradiation time (1 hour) was required to generate a sufficient NO concentration that can kill E. coli. By the end of the experiment (2 hours), 23% of total NO was recovered.

In addition, a control experiment investigated NO release from hydrogel 4 in the absence of triggers (UV or blue light). No peaks corresponding to the azo compound at 548 nm were observed, indicating that hydrogel 4 does not leach NO and that light is essential for NO release.

Properties of Hydrogel 4 after NO was Released

To investigate whether the physical properties of nitrobenzene-appended short peptide hydrogel 4 were affected by NO release, hydrogels that had been irradiated (λ=440-450 nm) for 2 hours were investigated using the vial inversion test, rheology, and AFM.

From the vial inversion test, performed after NO was released, hydrogel 4 still exhibited the characteristics of a stable hydrogel (FIG. 21a). In rheology, UV irradiation of hydrogel 4 led to a significant decrease in G′ value from 10.2 kPa to 4.9 kPa in the FST (FIG. 21b). However, the G′ value of hydrogel 4 was still an order of magnitude higher than its G″ value. This indicates that hydrogel 4 was able to maintain its stable characteristics after being exposed to blue light irradiation.

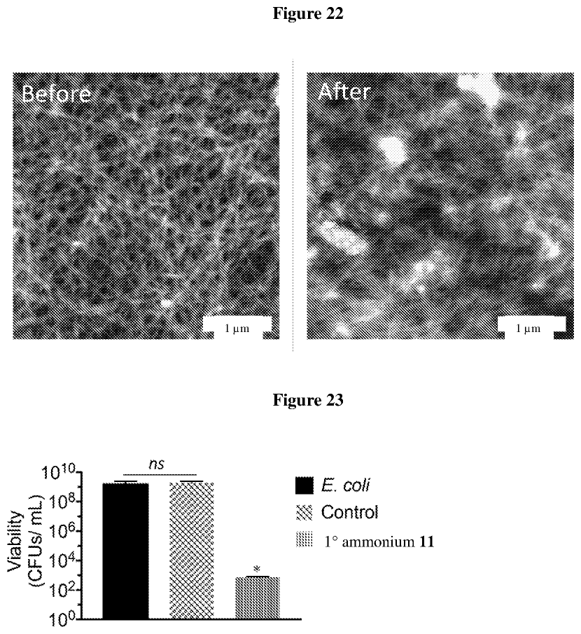

Aside from a slightly smaller fiber bundle, with diameter of 68±6 nm, AFM images taken from the hydrogel 4 which had been irradiated for 2 hours (FIG. 22) showed similar fiber morphology to that of the native hydrogel (before irradiation). Due to its capability of retaining its properties after irradiation, hydrogel 4 has high potential to be used for topical application.

The above results show that nitrobenzene, as a class of NO donor, can be incorporated into an anthranilamide-based short peptide. The facile synthesis allows nitrobenzene-appended anthranilamide-based short peptides to be synthesised on a gram scale in high yield. In addition, short peptide 4 successfully formed hydrogels at physiological pH (as well as at high pH). The resulting hydrogel exhibited desirable mechanical properties to be used for topical application, and low toxicity against HEK 293T cells. Importantly, hydrogel 4 demonstrated light-dependent NO generation and showed effective bactericidal activities against E. coli K12. These attributes make hydrogel 4 a promising candidate for topical antibacterial application.

### Example 7—Fibers

The fibers were further investigated for their antibacterial properties.

### Preparation

Fibers were prepared using the same procedure as for making the hydrogels. In addition, concentrations less than the CGC (i.e. <1 mg/mL) could be prepared to provide the fibers. Once a hydrogelator compound is dissolved in a solution and left for some time the solution will form fibers, even at concentrations lower than CGC.

Release of Cationic Short Peptides from Hydrogel

The mechanism of antibacterial action for most hydrogels is still not fully understood, however one plausible pathway relates to the release of active substance from the hydrogel. To examine this hypothesis, the released solution from hydrogels was initially investigated using UV/Vis spectroscopy.

Hydrogel 11 was made at 1% w/v with a final volume of 1 mL. The resulting hydrogel was left to stand overnight at room temperature. Phosphate buffer solution (PBS, 1 mL) was added gently from the side wall to avoid physical fractures. Subsequently, the vial was incubated at 37° C., where 1 mL of PBS was sampled at each time point and replaced with fresh PBS. Samples were subjected to 10× dilution before analysis using UV-Vis spectroscopy. This experiment was performed in triplicate.

### Results

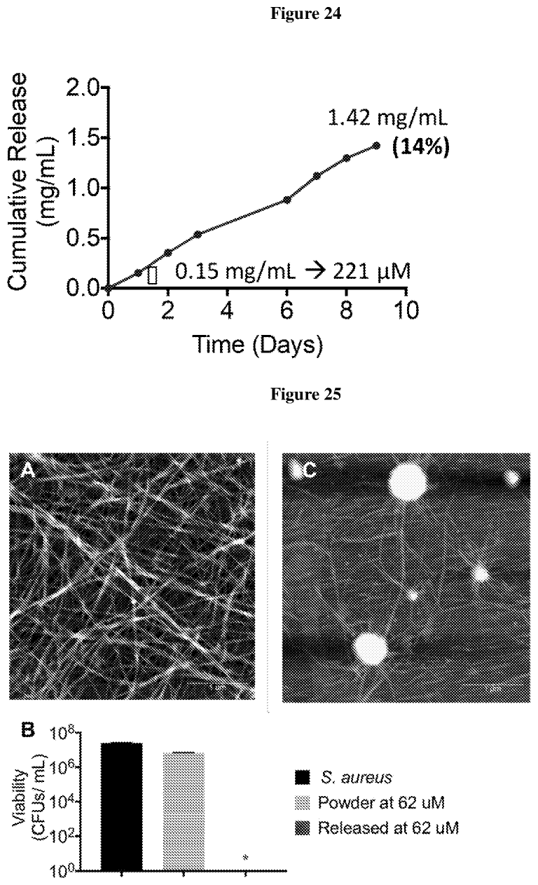

The UV/Vis spectra showed peaks which centered at 235 nm and 290 nm, suggesting that the cationic short peptide was being released from the hydrogel, which could account for its antibacterial activity. After incubation at 37° C. for 24 hours, 0.15 mg/mL (221 μM) was quantified to be released from primary ammonium hydrogel 11. Furthermore, a continuous release profile was observed over 9 days with ˜14% of total peptide being released at the end of the experiment (FIG. 24).

### AFM

AFM was performed on the released solution to examine the morphology of the released compound from hydrogel 11. Although the concentration of the compound released was below their CGC (1 mg/mL), fibers with diameter of 47±4.9 nm were observed (FIG. 25a).

### Antibacterial Assays

To confirm their antibacterial efficacy, the released fibers from day 1 and day 9 were subjected to a standard microdilution protocol.

To further investigate their antibacterial activity, the released fibers from hydrogel 11 were tested against S. aureus 38. The solutions sampled from day 1 and day 9 were adjusted to obtained final concentrations of 125, 62.5, 31.25, and 18 μM. Then, 100 μL of these solutions were transferred to a 96 well plate. On the other hand, short cationic peptide mimic 11, that did not form a hydrogel, was prepared by dissolving a known amount of peptide mimic 11 in DMSO to give a 20 mM stock solution which was then diluted with LB to make up concentrations of 125, 62.5, 31.25, and 18 μM. 100 μL of each of these peptide solutions was also transferred to a 96 well plate.

Subsequently, 100 μL of a bacteria solution, adjusted to 10⁶ CFUs/mL was added to each well. Two controls were used: one containing 100 μL of LB and 100 μL of PBS, and the other containing 100 μL of bacteria solution and 100 μL of PBS. The plate was then incubated at 37° C. for 24 hours. The following day, 20 μL solution from each well was subjected to a serial dilution then transferred onto agar plates followed by incubation at 37° C. for another 24 hours. MBC value was determined as the lowest concentration that inhibited S. aureus growth on the agar plate.

### Results

Complete kill of S. aureus was observed from the released solution obtained from each day of the experiment, with the minimum bactericidal concentration (MBC) calculated to be 62 μM (FIG. 25b). MBC is the minimum antibacterial agent required to kill bacteria as opposed to being bacteriostatic.

Surprisingly, the antibacterial activity of anthranilamide-based ultra-short cationic peptides appears to rely on the self-assembled morphology of the fibers. At the same concentration (62 μM), short cationic peptide 11 (as a free compound) did not show significant bacteria reduction (<1 Log₁₀ CFU/mL) (FIG. 25b, middle column). AFM images obtained for short cationic peptide 11, which was prepared without forming the hydrogel (prepared with 5% DMSO:water), showed a mixture of thin fibers with a lack of junction zones, and spheroidal aggregates (FIG. 25c).

It is to be understood that, if any prior art publication is referred to herein, such reference does not constitute an admission that the publication forms a part of the common general knowledge in the art, in Australia or any other country.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A compound of formula (I): 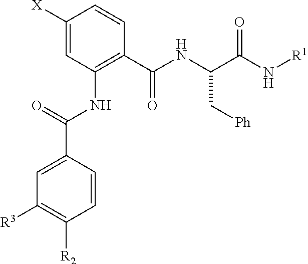
wherein:
R¹ is 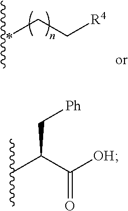
R⁴ is 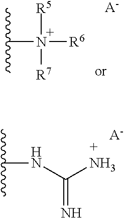
R⁵, R⁶, and R⁷ are, independently, H or CH₃;
A⁻ is Cl⁻, Br⁻, I⁻, CH₃C(O)O⁻, CF₃C(O)O⁻, or GdL⁻;
GdL⁻ is 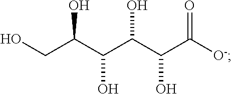
X is H, F, Cl, or Br;
R² is NO₂;
R³ is CH₃ or CF₃; or
R² and R³, when taken together with the carbon atoms to which they are attached, form a benzene ring, wherein the resulting naphthalene is optionally substituted with a group selected from the group consisting of halo, CN, CF₃, OCF₃, OCH₃, OCH₂CH₃, N(CH₃)₂, N(CH₂CH₃)₂, N(CH₃)(CH₂CH₃), COCH₃, COCH₂CH₃, OCOCH₃, SCH₃, SCH₂CH₃, C₁-C₄ alkyl, C₂-C₄ alkenyl, and C₂-C₄ alkynyl; and
n is 1, 2 or 3.

2. The compound according to claim 1,
wherein R¹ is 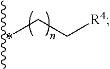
and
R² and R³, when taken together with the carbon atoms to which they are attached, form a benzene ring, wherein the resulting naphthalene is optionally substituted with a group selected from the group consisting of halo, CN, CF₃, OCF₃, OCH₃, OCH₂CH₃, N(CH₃)₂, N(CH₂CH₃)₂, N(CH₃)(CH₂CH₃), COCH₃, COCH₂CH₃, OCOCH₃, SCH₃, SCH₂CH₃, C₁-C₄ alkyl, C₂-C₄ alkenyl, and C₂-C₄ alkynyl.

3. The compound according to claim 1,
wherein R¹ is 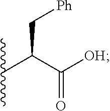 [SMILES: *[C@@H](Cc1ccccc1)C(=O)O]
and
R² is NO₂.

4. An antibacterial hydrogel, comprising water and a compound of formula (I): 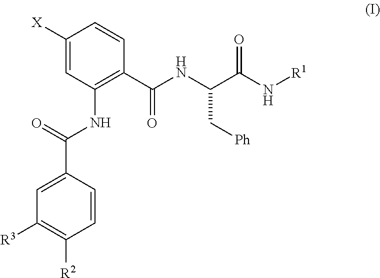
wherein:
R¹ is 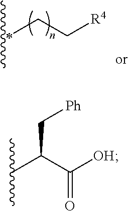
R⁴ is 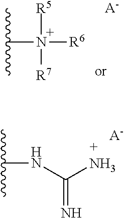
R⁵, R⁶, and R⁷ are, independently, H or CH₃;
A⁻ is Cl⁻, Br⁻, I⁻, CH₃C(O)O⁻, CF₃C(O)O⁻, or GdL⁻;
GdL⁻ is 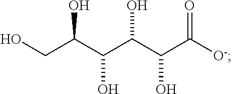
X is H, F, Cl, or Br;
R² is NO₂;
R³ is CH₃ or CF₃; or
R² and R³, when taken together with the carbon atoms to which they are attached, form a benzene ring, wherein the resulting naphthalene is optionally substituted with a group selected from the group consisting of halo, CN, CF₃, OCF₃, OCH₃, OCH₂CH₃, N(CH₃)₂, N(CH₂CH₃)₂, N(CH₃)(CH₂CH₃), COCH₃, COCH₂CH₃, OCOCH₃, SCH₃, SCH₂CH₃, C₁-C₄ alkyl, C₂-C₄ alkenyl, and C₂-C₄ alkynyl; and
n is 1, 2 or 3.

5. The antibacterial hydrogel according to claim 4,
wherein R¹ is 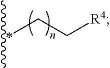
and
R² and R³, when taken together with the carbon atoms to which they are attached, form a benzene ring, wherein the resulting naphthalene is optionally substituted with a group selected from the group consisting of halo, CN, CF₃, OCF₃, OCH₃, OCH₂CH₃, N(CH₃)₂, N(CH₂CH₃)₂, N(CH₃)(CH₂CH₃), COCH₃, COCH₂CH₃, OCOCH₃, SCH₃, SCH₂CH₃, C₁-C₄ alkyl, C₂-C₄ alkenyl, and C₂-C₄ alkynyl.

6. The antibacterial hydrogel according to claim 5, wherein the hydrogel has a pH of between about 4.0 and about 6.0.

7. The antibacterial hydrogel according to claim 5 or claim 6, wherein the hydrogel, at a concentration of 1% w/v, exhibits a bacterial reduction of between about 3.0 Log₁₀ and about 9.0 Log₁₀.

8. The antibacterial hydrogel according to claim 7, wherein the bacterial reduction is in relation to Staphylococcus aureus.

9. The antibacterial hydrogel according to any one of claims 5 to 8, wherein the hydrogel has a linear viscoelastic region (LVER) of between about 0.98% and about 2.01%.

10. The antibacterial hydrogel according to claim 4,
wherein R¹ is 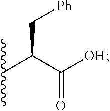 [SMILES: *[C@@H](Cc1ccccc1)C(=O)O]
and
R² is NO₂.

11. The antibacterial hydrogel according to claim 10, wherein the hydrogel has a pH of between about 5.0 and about 8.0.

12. The antibacterial hydrogel according to claim 10 or claim 11, wherein the hydrogel, under UV or blue light irradiation, generates nitric oxide (NO).

13. The antibacterial hydrogel according to any one of claims 10 to 12, wherein the hydrogel has a linear viscoelastic region (LVER) of between about 1.20% and about 1.40% at a pH of between about 5.0 and about 8.0.

14. The antibacterial hydrogel according to any one of claims 4 to 13, wherein the hydrogel has a critical gel concentration of between about 0.1% w/v and about 0.3% w/v.

15. The antibacterial hydrogel according to any one of claims 4 to 14, wherein the hydrogel has a G′/G″ ratio of between about 10 and 18.

16. The antibacterial hydrogel according to any one of claims 4 to 15, further comprising a salt.

17. The antibacterial hydrogel according to claim 16, wherein the salt is sodium chloride.

18. An antibacterial fiber, comprising a compound according to any one of claims 1 to 3.

19. Use of the compound according to any one of claims 1 to 3, the hydrogel according to any one of claims 4 to 17, or the fiber according to claim 18, in a barrier material or in an antibacterial carrier material for organ transplantation.

20. An antibacterial composition for disinfecting a surface, comprising the compound according to any one of claims 1 to 3, the hydrogel according to any one of claims 4 to 17, or the fiber according to claim 18.

21. The composition according to claim 20, wherein the composition is applied by spray or by dip coating.

22. An antibacterial coating for an article, comprising the compound according to any one of claims 1 to 3, the hydrogel according to any one of claims 4 to 17, or the fiber according to claim 18.

23. A wound dressing for the treatment or prevention of a bacterial infection, comprising the compound according to any one of claims 1 to 3, the hydrogel according to any one of claims 4 to 17, or the fiber according to claim 18.

24. A method of preventing or treating a bacterial infection, comprising topical administration to a subject a therapeutically effective amount of the compound according to any one of claims 1 to 3, the hydrogel according to any one of claims 4 to 17, or the fiber according to claim 18.
